(19) United States
(12) Patent Application Publication
(10) Pub. No.: US 2021/0179578 A1
(43) Pub. Date: Jun. 17, 2021

(54) OX1 ANTAGONISTS

(71) Applicant: Heptares Therapeutics Limited, Cambridge, Cambridgeshire (GB)

(72) Inventors: John Andrew Christopher, Cambridge (GB); Sarah Joanne Bucknell, Cambridge (GB); Miles Stuart Congreve, Cambridge (GB); Benjamin Gerald Tehan, Cambridge (GB); Rebecca Helen Nonoo, Cambridge (GB); Giles Albert Brown, Cambridge (GB); Nigel Alan Swain, Cambridge (GB); Mark Mills, Nottingham (GB)

(21) Appl. No.: 16/760,807

(22) PCT Filed: Nov. 5, 2018
(86) PCT No.: PCT/GB2018/053200

§ 371 Date: Apr. 30, 2020

(30) Foreign Application Priority Data
Nov. 3, 2017 (GB) 1718256.9

(51) Int. Cl. C07D 401/04; C07D 243/04

(52) U.S. Cl. CPC C07D 401/04; C07D 243/04

(57) ABSTRACT

The disclosures herein relate to novel compounds of formula (1) and salts thereof, wherein X; Y; R₁; R₂; R₃; R₄; R₅; R₆; R₇ and R₈ are defined herein, and their use in treating, preventing, ameliorating, controlling or reducing the risk of neurological or psychiatric disorders associated with orexin receptors.

This application relates to novel compounds and their use as orexin receptor antagonists. Compounds described herein may be useful in the treatment or prevention of diseases in which orexin receptors are involved. The application is also directed to pharmaceutical compositions comprising these compounds and the manufacture and use of these compounds and compositions in the prevention or treatment of such diseases in which orexin receptors are involved.

### BACKGROUND OF THE INVENTION

Neuroanatomical, immunohistochemical and pharmacological evidence suggests that the orexins play important roles in the regulation of a wide variety of physiological functions and complex behaviours. Orexinergic (aka hypocretinergic) neurones are located in the lateral hypothalamus and perifornical area of the mammalian mid-brain and from here they project widely throughout the central nervous system and spinal cord via ascending and descending pathways (Peyron et al, J. Neurosci., 1998, 18, 9996-10015). Orexinergic neurons project to forebrain structures involved in cognition and emotion, such as the cortex, amygdala, hippocampus and septum. In addition, they strongly target ascending neuronal systems involved in the control of arousal, movement, sleep wake cycles and reward processing, notably the noradrenergic locus coruleus, serotonergic Raphe nuclei, dopaminergic ventral tegmental area and Substantia nigra, cholinergic Pedunculopontine nuclei and histaminergic Tuberomammillary bodies. Similarly, orexinergic neurons project to brain areas involved in the control of feeding in the hypothalamus such as MCH, NPY and leptin positive neurons in the arcuate nucleus. Orexinergic innervation of the dorsal horn of the spinal cord has been reported and evidence for the presence of orexin in peripheral tissues including plasma, sympathetic ganglia, myenteric plexus, endocrine cells of the gastrointestinal tract, adrenal gland, pancreas, placenta, testes, pituitary, kidney, adrenal gland, thyroid and lung amongst others suggest important roles in the regulation of a wide variety of physiological functions.

The orexin peptides orexin A and orexin B are produced in the hypothalamus and exert their effect through activation of the orexin receptors: orexin 1 (OX₁R) and orexin 2 (OX₂R). OX₁R and OX₂R share 64% amino acid identity. Orexin A and B have equal affinity for OX₂R receptors whilst the affinity of orexin B is around one order of magnitude lower at the OX₁R receptor (Sakurai et al, Cell, 1998, 92, 573-585). The orexin receptors are predominately linked to G_{q/11} G proteins with activation of the receptors leading to activation of the phospholipase C pathway triggering influx of extracellular sodium and calcium and depolarisation of neurones.

Whilst OX₁R and OX₂R show some overlapping distribution, they largely show distinct (but complementary) patterns of distribution within the central nervous system which suggests that each receptor subtype plays a different physiological role. Outside of the hypothalamus the highest levels of OX₁R are found in the locus coreleus, tenia tecta, hippocampal formation, dorsal raphe nucleus—areas which have been linked to anxiety, arousal, stress and panic disorders and reward-seeking behaviour. OX₂R is found in the cerebral cortex, nucleus accumbens, subthalamic nucleus, paraventricular thalamic nuclei, anterior pretectal nucleus raphe nuclei and tuberomammillary bodies (Tsujino and Sakurai, Pharmacol. Rev., 2009, 61, 162-176). Orexin receptors are also expressed in peripheral tissues, for example OX₁R can be found in the kidney, testis, thyroid, ovaries, jejunum and adrenal glands (Jöhren et al, Endocrinology, 2001, 142, 3324-3331).

There is substantial preclinical evidence supporting the hypothesis that OX₁R receptor antagonists are efficacious in preclinical models of drug abuse (Boutrel et al, Front. Behav. Neurosci., 2013, 7, 1-10). Intravenous self-administration of cocaine is attenuated in OX₁R knock out mice (Hollander et al, Front. Behav. Neurosci., 2012, 6, 1-9; Muschamp et al, Proc. Nat. Acad. Sci. U.S.A., 2014, e1648-e1655). Intracerebroventricular administration of orexin A leads to a dose-related reinstatement of cocaine seeking and dramatically elevates intracranial self-stimulation thresholds in rodent self-administration studies (Boutrel et al, Proc. Nat. Acad. Sci. U.S.A., 2005, 102, 19168-19173). Administration of a partially selective OX₁R antagonist SB-334867 decreases alcohol (Lawrence et al, Br. J. Pharmacol., 2006, 148, 752-759) and nicotine (Hollander et al, Proc. Nat. Acad. Sci. U.S.A., 2008, 105, 19480-19485) self-administration in rats. In rats intraperitoneal administration of SB-334867 has also been demonstrated to significantly reduce opiate withdrawal symptoms (Laorden et al, PLoS One, 2012, 7, e36871).

Selective OX₁ receptor antagonists have potential utility therefore to treat a number of Substance Related and Addictive Disorders such as Opioid Use Disorder (including but not limited to Opioid Use Disorder, Opioid Intoxication, Opioid withdrawal, Other Opioid Induced Disorder and Unspecified Opioid Related Disorder): Stimulant Related disorders (including but not limited to Stimulant Use Disorder, Stimulant Intoxication, Stimulant Withdrawal, Other Stimulant Induced Disorders and Unspecified Stimulant Related Disorder where such disorders are associated with the abuse of stimulant drugs exemplified but not limited to cocaine or related structures and, amphetamine-like substances): Caffeine Related Disorders (including but not limited to Caffeine intoxication, Caffeine withdrawal, and Unspecified Caffeine Related disorders): Tobacco Related Disorders (including but not limited to Tobacco Use Disorder, Tobacco Withdrawal, Other Tobacco Induced Disorders and Unspecified Tobacco Related Disorder where such disorders are associated with the use of Tobacco, Tobacco products or the inhalation of nicotine and related compounds): Alcohol Use Disorder, Alcohol Intoxication, Alcohol Withdrawal and Unspecified Alcohol Related Disorder: Cannabis Related Disorders (including but not limited to Cannabis Use Disorder, Cannabis Intoxication, Cannabis Withdrawal, and Unspecified Cannabis Related Disorders whether associated with the use of cannabis, cannabis extracts or synthetic cannabinoids): Hallucinogen Related Disorders (including but not limited to Phencyclidine Use Disorder, Phencyclidine intoxication, Other Hallucinogen Use Disorder, Hallucinogen Persisting Perception Disorder and Unspecified Hallucinogen Related Disorder): Inhalant Related Disorders (including but not limited to Inhalant Use Disorder, Inhalant Intoxication, Other Inhalant Induced Disorders and Unspecified Inhalant Related Disorder where such disorders are associated with the use of compounds such as volatile hydrocarbons, nicotine or nicotine derivatives): Sedative, Hypnotic or Anxiolytic Related Disorders (including but not limited to Use Disorder, Intoxication and Withdrawal). Selective OX₁ receptor antagonists also have potential utility to treat a number of Non-Substance Related Disorders such as Gambling Disorder, Internet Gaming Disorder or addiction to sex or internet use.

There is emerging evidence of the orexin systems involvement in anxiety, panic and fear associated learning. Orexin neurones in perifornical and lateral hypothalamic areas are highly reactive to anxiogenic stimuli and optogenetic stimulation of orexin neurons in rodents increases anxiety-like states (Heydend et al, Physiol. Behav., 2013, 130, 182-190). Using two models of panic (sodium lactate infusion and CO₂ challenge models) blockade of OX₁R with the highly selective antagonist JNJ-54717793 attenuated panic-like behaviours and cardiovascular responses in rats without altering baseline locomotor or autonomic activity and without sedation (Bonaventure et al, Front. Pharmacol., 2017, 8, 1-13). In a study of 53 medication-free patients with suicidal behaviour increased orexin levels were observed in the cerebral spinal fluid versus control (Johnson et al, Nat. Med., 2010, 16, 111-118) suggesting a link with orexin levels and anxiety.

Selective OX₁ receptor antagonists therefore have potential utility to treat Anxiety Disorders (including but not limited to Separation Anxiety Disorder, Specific Phobia, Social Anxiety Disorder (Social Phobia), Panic Disorder, Agoraphobia, Generalized Anxiety Disorder, Substance/Medication Induced Anxiety Disorder and Anxiety Disorder due to Another Medical Condition): Disruptive Mood Dysregulation Disorder, Major Depressive Disorder particularly, but not exclusively, when specified with Anxious Distress, mixed features, atypical features, peripartum onset or seasonal pattern: Persistent Depressive Disorder (Dysthymia) particularly, but not exclusively, when specified with Anxious Distress, mixed features, atypical features, peripartum onset or seasonal pattern: Premenstrual Dysphoric Disorder, Substance/Medication-Induced Depressive Disorder, Other Specified Depressive Disorder or Unspecified Depressive Disorder. Similarly, OX₁ receptor antagonists have potential utility to treat the symptoms of Bipolar and Related Disorders (including but not restricted to Bipolar I Disorder and Bipolar II Disorder, particularly, but not exclusively, when these are specified with Anxious Distress, Cyclothymic Disorder, Substance/Medication-induced Bipolar and Related Disorder or Bipolar and Related Disorder due to Another Medical Condition): Schizophrenia Spectrum and other disorders (including but not limited to Schizotypal Personality, Delusional Disorder, Schizophreniform Disorder, Schizophrenia, Schizoaffective Disorder, and Substance/Medication-Induced Psychotic Disorder).

It has been shown that orexin plays a role in mediating the extinction of fear in animal models of stress and trauma (Flores et al, Trends Neurosci., 2015, 38, 550-559). OX₁ receptor antagonists therefore have potential utility to treat conditions associated with trauma and stress including but not limited to Post Traumatic Stress Disorder, Acute Stress Disorder, Adjustment Disorders particularly, but not exclusively, when specified with Anxiety or with Mixed Anxiety and Depressed Mood: Obsessive Compulsive and Related Disorders including but not limited to Obsessive Compulsive Disorder, Body Dysmorphia, Trichlotillomania, Excoriation and Obsessive Compulsive and Related Disorders due to another medical condition: Feeding and Eating Disorders including but not limited to Binge Eating Disorder, Anorexia Nervosa, Bulimia Nervosa, Cachexia, Obesity and Prader Willi syndrome.

Some Sleep-Wake Disorders may also be treated with selective OX₁ receptor antagonists and these disorders include but are not limited to Insomnia Disorder, Rapid Eye Movement Disorder, Sleep disturbances associated with diseases, Sleep Apnoea, Narcolepsy and Circadian Rhythm Sleep-Wake Disorders. Similarly, Neurodegenerative disorders (including but not limited to Parkinson's Disease, Alzheimer's Disease, Dementia, Lewy-body Dementia, Frontotemporal Dementia, Multiple System Atrophy, Perry Syndrome, Klein-Levin Syndrome, Amyotrophic Lateral Sclerosis, Niemann-Pick disease and Multiple Sclerosis): Behavioural Symptoms of neurodegenerative and other disorders (such as Positive Symptoms, Psychosis, Agitation, Anhedonia, Apathy): Movement disorders (such as Akinesias, Dyskinesia, Drug-induced Parkinsonism, Dystonia): other CNS disorders (such as Affective Neurosis, Delirium, Sexual Dysfunction, Psychosexual Dysfunction, Rett Syndrome, Attention-Deficit Disorder, Attention-Deficit Hyperactivity Disorder, Autism (including but not limited to autism spectrum disorder, autism spectrum condition, atypical autism, classic autism, Kanner autism, pervasive developmental disorder, high-functioning autism and Asperger syndrome), Fragile X syndrome, Disruptive behaviour disorder, Severe mental retardation, Vomiting.

Pathological conditions showing reduced metabolic activity or a decrease in resting energy expenditure as a percentage of total fat-free mass may be amenable to treatment with selective OX₁ receptor antagonists, such conditions include, but are not limited to Diabetes, impaired glucose tolerance; Cardiovascular Disease including but not limited to Acute and Congestive Heart Failure, Hypotension, Hypertension, Angina Pectoris, Myocardial infarction.

Hypothalamic/pituitary disorders may be amenable to treatment with selective OX₁ receptor antagonists, including but not limited to Cushing's Syndrome/disease, Basophile adenoma, Prolactinoma, hyperprolactinemia, Hypophysis Tumour/adenoma, Hypothalamic diseases, Froehlich's syndrome, Adrenohypophysis disease, Hypophysis disease, Adrenohypophysis hypofunction, Adrenohypophysis hyperfunction, Hypothalamic hypogonadism, Kallman's syndrome (anosmia, hyposmia), functional or psychogenic Amenorrhea, Hypopituitarism, Hypothalamic Hypothyroidism, Hypothalamic-adrenal dysfunction, Idiopathic Hyperprolactinemia, Hypothalamic Disorders of growth hormone deficiency, Idiopathic growth deficiency, Dwarfism, Gigantism, Acromegaly.

Pain disorders may be amenable to treatment with selective OX receptor antagonists, including but not limited to Neuropathic pain, Restless Leg Syndrome, Migraine, Cluster headache, Tension-type headache, Trigeminal autonomic Cephalalgias, Hemicrania Continua, Trigeminal neuralgia, other headache disorders, pain, enhanced or exaggerated sensitivity to pain such as Hyperalgesia, Causalgia, and Allodynia, Acute pain, Burn pain, Atypical facial pain, Back pain, Complex regional pain syndrome I and II, Arthritic pain, Sports injury pain, pain related to infection e.g. HIV, Post-chemotherapy pain, Post-stroke pain, Post-operative pain, neuralgia, conditions associated with Visceral pain such as Irritable bowel syndrome, and Angina pain; Inflammatory disorders (including Inflammatory bowel disease), Renal/urinary disorders (including urinary retention, benign prostatic hypertrophy, chronic renal failure, renal disease); Respiratory disorders (such as Chronic obstructive pulmonary disease, Asthma).

Orexin-A/OX₁ receptor interactions have been shown to modulate activity of the androgen receptor (Valiante et al, Biochem. Biophys. Res Comm., 2015, 464, 1290-1296) which regulates the onset and progression of prostate cancer, suggesting that selective OX₁ receptor antagonists have potential therapeutic benefit in treating some forms of cancer including, but not limited to, Prostate cancer, Liver Cancer, Colon cancer, Endometrial cancer, Pancreatic cancer and cancers associated with other organs of the body including the central nervous system and peripheral nervous system.

WO02/100327 discloses substituted 1-benzazepines and derivatives thereof and their use as anti-infective agents.

### SUMMARY OF THE INVENTION

The invention relates to novel compounds. The invention also relates to the use of novel compounds as antagonists of orexin receptor OX₁. The invention further relates to the use of novel compounds in the manufacture of medicaments for use as orexin receptor OX₁ antagonists or for the treatment of orexin system dysfunction. The invention further relates to compounds, compositions and medicaments for the treatment of neurological or psychiatric disorders. Embodiments of the invention may be compounds according to the formula (1)

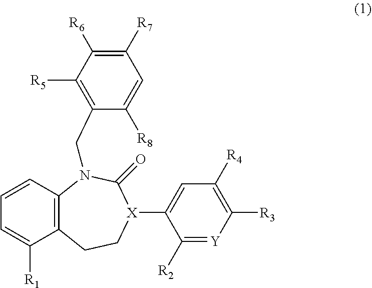

and salts thereof, wherein

### X is CH or N;

### Y is CH or N;

### R₁ is H or F;

R₂ is H, C₁-C₃ alkyl or C₁-C₃ alkoxy;
R₃ is C₁-C₃ alkyl or C₁-C₃ alkoxy;
R₄ is SO(n)CH₃ where n is 0-2 or C₁-C₃ alkyl or C₁-C₃ alkoxy where the alkyl or alkoxy groups are optionally substituted with one or more fluorine atoms;
R₅ is H, halo, cyano, C₁-C₃ alkyl or C₁-C₃ alkoxy;
R₆ is H, halo, cyano, C₁-C₃ alkyl or C₁-C₃ alkoxy;
R₇ is H, halo, cyano, C₁-C₃ alkyl or C₁-C₃ alkoxy; wherein the C₁-C₃ alkyl group can be substituted with C₁-C₃ alkoxy; and

### R₈ is H or F.

Specific embodiments include the following substituents, which can be combined in any particular combination without limitation:

X can be CH. X can be N.

Y can be CH. Y can be N.

R₁ can be H. R₁ can be F.

R₂ can be H. R₂ can be C₁-C₃ alkyl. R₂ can be C₁-C₃ alkoxy. R₂ can be CH₃. R₂ can be OCH₃.

R₃ can be C₁-C₃ alkyl. R₃ can be C₁-C₃ alkoxy. R₃ can be CH₃. R₃ can be OCH₃. R₃ can be CD₃. R₃ can be OCD₃.

R₄ can be SO(n)CH₃ where n is 0-2. R₄ can be SCH₃. R₄ can be S(O)CH₃. R₄ can be SO₂CH₃. R₄ can be C₁-C₃ alkyl. R₄ can be C₁-C₃ alkyl where the alkyl group is substituted with one or more fluorine atoms. R₄ can be C₁-C₃ alkoxy. R₄ can be C₁-C₃ alkoxy where the alkoxy group is substituted with one or more fluorine atoms. R₄ can be CF₂CH₃. R₄ can be OCF₂H.

R₅ can be H. R₅ can be halo. R₅ can be cyano. R₅ can be C₁-C₃ alkyl. R₅ can be C₁-C₃ alkoxy. R₅ can be F. R₅ can be CH₃.

R₆ can be H. R₆ can be halo. R₆ can be cyano. R₆ can be C₁-C₃ alkyl. R₆ can be C₁-C₃ alkoxy. R₆ can be CN. R₆ can be F.

R₇ can be H. R₇ can be halo R₇ can be cyano R₇ can be C₁-C₃ alkyl. R₇ can be or C₁-C₃ alkoxy. R₇ can be C₁-C₃ alkyl substituted with C₁-C₃ alkoxy. R₇ can be F. R₇ can be Cl. R₇ can be Br. R₇ can be OCH₃. R₇ can be OCH(CH₃)₂. R₇ can be OCD₃. R₇ can be CH₂OCH₃. R₇ can be CH₂OCD₃. R₇ can be CH₂CH₂CH₃.

R₈ can be H. R₈ can be F.

The compounds may be for use as antagonists of orexin receptor OX₁. The compounds may be used in the manufacture of medicaments. The compounds or medicaments may be for use in treating, preventing, ameliorating, controlling or reducing the risk of neurological or psychiatric disorders.

The compounds or medicaments may be for use in the treatment or prevention of substance related and addictive disorders (including opioid use disorder, opioid intoxication, opioid withdrawal, other opioid induced disorder, unspecified opioid related disorder, stimulant use disorder, stimulant intoxication, stimulant withdrawal, other stimulant induced disorders, unspecified stimulant related disorder, caffeine related disorders, caffeine intoxication, caffeine withdrawal, unspecified caffeine related disorders, tobacco related disorders, tobacco use disorder, tobacco withdrawal, other tobacco induced disorders, unspecified tobacco related disorder, alcohol use disorder, alcohol intoxication, alcohol withdrawal, unspecified alcohol related disorder, cannabis related disorders, cannabis use disorder, cannabis intoxication, cannabis withdrawal, unspecified cannabis related disorders, hallucinogen related disorders, phencyclidine use disorder, phencyclidine intoxication, other hallucinogen use disorder, hallucinogen persisting perception disorder, unspecified hallucinogen related disorder, inhalant related disorders, inhalant use disorder, inhalant intoxication, other inhalant induced disorders, unspecified inhalant related disorder, sedative, hypnotic or anxiolytic related disorders (including use disorder, intoxication and withdrawal), gambling disorder, internet gaming disorder, addiction to sex or internet use), anxiety disorders (including separation anxiety disorder, specific phobia, social anxiety disorder (social phobia), panic disorder, agoraphobia, generalized anxiety disorder, substance/medication induced anxiety disorder, anxiety disorder due to another medical condition), disruptive mood dysregulation disorder, major depressive disorder (including when specified with anxious distress, mixed features, atypical features, peripartum onset or seasonal pattern), persistent depressive disorder (dysthymia) (including when specified with anxious distress, mixed features, atypical features, peripartum onset or seasonal pattern), premenstrual dysphoric disorder, substance/medication-induced depressive disorder, other specified depressive disorder, unspecified depressive disorder bipolar and related disorders (including bipolar I disorder and bipolar II disorder, particularly, but not exclusively, when these are specified with anxious distress, cyclothymic disorder, substance/medication-induced bipolar and related disorder or bipolar and related disorder due to another medical condition), schizophrenia spectrum and other disorders (including schizotypal personality, delusional disorder, schizophreniform disorder, schizophrenia, schizoaffective disorder, and substance/medication-induced psychotic disorder), conditions associated with trauma and stress (including post traumatic stress disorder, acute stress disorder, adjustment disorders (including when specified with anxiety or with mixed anxiety and depressed mood), obsessive compulsive and related disorders (including obsessive compulsive disorder, body dysmorphia, trichlotillomania, excoriation and obsessive-compulsive and related disorders due to another medical condition), feeding and eating disorders (including binge eating disorder, anorexia nervosa, bulimia nervosa, cachexia, obesity, Prader Willi syndrome)), sleep-wake disorders, neurodegenerative disorders (including dementia), behavioural symptoms of neurodegenerative and other disorders, movement disorders, diabetes, impaired glucose tolerance, cardiovascular disease, diseases related to modulation of sympathetic outflow including hypertension, hypothalamic/pituitary disorders, neuropathic pain, restless leg syndrome, migraine, cluster headache, tension-type headache, trigeminal autonomic cephalalgias, hemicrania continua, trigeminal neuralgia, other headache disorders, hyperalgesia, pain, hyperalgesia, causalgia, and allodynia, acute pain, burn pain, atypical facial pain, back pain, complex regional pain syndrome I and II, arthritic pain, sports injury pain, pain related to infection, irritable bowel syndrome, angina pain, inflammatory disorders, renal/urinary disorders, respiratory disorders, cancer (including prostate cancer, liver cancer, colon cancer, endometrial cancer, pancreatic cancer and cancers associated with other organs of the body including the central nervous system and peripheral nervous system).

The compounds or medicaments may be for use in the treatment or prevention of substance related and addictive disorders, post traumatic stress disorder, panic disorder, major depressive disorder with anxious distress, diseases related to modulation of sympathetic outflow including hypertension, pain, headache, cancer.

The compounds may be formulated as a pharmaceutical composition comprising the compounds and a pharmaceutically acceptable excipient.

The compounds may be produced using any chemical synthesis method.

### DETAILED DESCRIPTION OF THE INVENTION

The invention relates to novel compounds. The invention also relates to the use of novel compounds as antagonists of orexin receptor OX₁. The invention further relates to the use of novel compounds in the manufacture of medicaments for use as orexin receptor OX₁ antagonists or for the treatment of orexin system dysfunction. The invention further relates to compounds, compositions and medicaments which are selective OX₁ receptor antagonists.

The invention further relates to compounds, compositions and medicaments for the treatment of Substance Related and Addictive Disorders such as Opioid Use Disorder (including but not limited to Opioid Use Disorder, Opioid Intoxication, Opioid withdrawal, Other Opioid Induced Disorder and Unspecified Opioid Related Disorder): Stimulant Related disorders (including but not limited to Stimulant Use Disorder, Stimulant Intoxication, Stimulant Withdrawal, Other Stimulant Induced Disorders and Unspecified Stimulant Related Disorder where such disorders are associated with the abuse of stimulant drugs exemplified but not limited to cocaine or related structures and, amphetamine-like substances): Caffeine Related Disorders (including but not limited to Caffeine intoxication, Caffeine withdrawal, and Unspecified Caffeine Related disorders): Tobacco Related Disorders (including but not limited to Tobacco Use Disorder, Tobacco Withdrawal, Other Tobacco Induced Disorders and Unspecified Tobacco Related Disorder where such disorders are associated with the use of Tobacco, Tobacco products or the inhalation of nicotine and related compounds): Alcohol Use Disorder, Alcohol Intoxication, Alcohol Withdrawal and Unspecified Alcohol Related Disorder: Cannabis Related Disorders (including but not limited to Cannabis Use Disorder, Cannabis Intoxication, Cannabis Withdrawal, and Unspecified Cannabis Related Disorders whether associated with the use of cannabis, cannabis extracts or synthetic cannabinoids): Hallucinogen Related Disorders (including but not limited to Phencyclidine Use Disorder, Phencyclidine intoxication, Other Hallucinogen Use Disorder, Hallucinogen Persisting Perception Disorder and Unspecified Hallucinogen Related Disorder): Inhalant Related Disorders (including but not limited to Inhalant Use Disorder, Inhalant Intoxication, Other Inhalant Induced Disorders and Unspecified Inhalant Related Disorder where such disorders are associated with the use of compounds such as volatile hydrocarbons, nicotine or nicotine derivatives): Sedative, Hypnotic or Anxiolytic Related Disorders (including but not limited to Use Disorder, Intoxication and Withdrawal). Selective OX₁ receptor antagonists also have potential utility to treat a number of Non-Substance Related Disorders such as Gambling Disorder, Internet Gaming Disorder or addiction to sex or internet use.

The invention further relates to compounds, compositions and medicaments for the treatment of Anxiety Disorders (including but not limited to Separation Anxiety Disorder, Specific Phobia, Social Anxiety Disorder (Social Phobia), Panic Disorder, Agoraphobia, Generalized Anxiety Disorder, Substance/Medication Induced Anxiety Disorder and Anxiety Disorder due to Another Medical Condition): Disruptive Mood Dysregulation Disorder, Major Depressive Disorder particularly, but not exclusively, when specified with Anxious Distress, mixed features, atypical features, peripartum onset or seasonal pattern: Persistent Depressive Disorder (Dysthymia) particularly, but not exclusively, when specified with Anxious Distress, mixed features, atypical features, peripartum onset or seasonal pattern: Premenstrual Dysphoric Disorder, Substance/Medication-Induced Depressive Disorder, Other Specified Depressive Disorder or Unspecified Depressive Disorder. Similarly, OX₁ receptor antagonists have potential utility to treat the symptoms of Bipolar and Related Disorders (including but not restricted to Bipolar I Disorder and Bipolar II Disorder, particularly, but not exclusively, when these are specified with Anxious Distress, Cyclothymic Disorder, Substance/Medication-induced Bipolar and Related Disorder or Bipolar and Related Disorder due to Another Medical Condition): Schizophrenia Spectrum and other disorders (including but not limited to Schizotypal Personality, Delusional Disorder, Schizophreniform Disorder, Schizophrenia, Schizoaffective Disorder, and Substance/Medication-Induced Psychotic Disorder).

The invention further relates to compounds, compositions and medicaments for the treatment of conditions associated with trauma and stress including but not limited to Post Traumatic Stress Disorder, Acute Stress Disorder, Adjustment Disorders particularly, but not exclusively, when specified with Anxiety or with Mixed Anxiety and Depressed Mood: Obsessive Compulsive and Related Disorders including but not limited to Obsessive Compulsive Disorder, Body Dysmorphia, Trichlotillomania, Excoriation and Obsessive Compulsive and Related Disorders due to another medical condition: Feeding and Eating Disorders including but not limited to Binge Eating Disorder, Anorexia Nervosa, Bulimia Nervosa, Cachexia, Obesity and Prader Willi syndrome.

The invention further relates to compounds, compositions and medicaments for the treatment of Sleep-Wake Disorders. These disorders include but are not limited to Insomnia Disorder, Rapid Eye Movement Disorder, Sleep disturbances associated with diseases, Sleep Apnoea, Narcolepsy and Circadian Rhythm Sleep-Wake Disorders. Similarly, Neurodegenerative disorders (including but not limited to Parkinson's Disease, Alzheimer's Disease, Dementia, Lewy-body Dementia, Frontotemporal Dementia, Multiple System Atrophy, Perry Syndrome, Klein-Levin Syndrome, Amyotrophic Lateral Sclerosis, Niemann-Pick disease and Multiple Sclerosis): Behavioural Symptoms of neurodegenerative and other disorders (such as Positive Symptoms, Psychosis, Agitation, Anhedonia, Apathy): Movement disorders (such as Akinesias, Dyskinesia, Drug-induced Parkinsonism, Dystonia): other CNS disorders (such as Affective Neurosis, Delirium, Sexual Dysfunction, Psychosexual Dysfunction, Rett Syndrome, Attention-Deficit Disorder, Attention-Deficit Hyperactivity Disorder, Autism (including but not limited to autism spectrum disorder, autism spectrum condition, atypical autism, classic autism, Kanner autism, pervasive developmental disorder, high-functioning autism and Asperger syndrome), Fragile X syndrome, Disruptive behaviour disorder, Severe mental retardation, Vomiting.

The invention further relates to compounds, compositions and medicaments for the treatment of Pathological conditions showing reduced metabolic activity or a decrease in resting energy expenditure as a percentage of total fat-free mass. Such conditions include, but are not limited to Diabetes, impaired glucose tolerance; Cardiovascular Disease including but not limited to Acute and Congestive Heart Failure, Hypotension, Hypertension, Angina Pectoris, Myocardial infarction.

The invention further relates to compounds, compositions and medicaments for the treatment of Hypothalamic/pituitary disorders, including but not limited to Cushing's Syndrome/disease, Basophile adenoma, Prolactinoma, hyperprolactinemia, Hypophysis Tumour/adenoma, Hypothalamic diseases, Froehlich's syndrome, Adrenohypophysis disease, Hypophysis disease, Adrenohypophysis hypofunction, Adrenohypophysis hyperfunction, Hypothalamic hypogonadism, Kallman's syndrome (anosmia, hyposmia), functional or psychogenic Amenorrhea, Hypopituitarism, Hypothalamic Hypothyroidism, Hypothalamic-adrenal dysfunction, Idiopathic Hyperprolactinemia, Hypothalamic Disorders of growth hormone deficiency, Idiopathic growth deficiency, Dwarfism, Gigantism, Acromegaly.

The invention further relates to compounds, compositions and medicaments for the treatment of Pain disorders, including but not limited to Neuropathic pain, Restless Leg Syndrome, Migraine, Cluster headache, Tension-type headache, Trigeminal autonomic Cephalalgias, Hemicrania Continua, Trigeminal neuralgia, other headache disorders, pain, enhanced or exaggerated sensitivity to pain such as Hyperalgesia, Causalgia, and Allodynia, Acute pain, Burn pain, Atypical facial pain, Back pain, Complex regional pain syndrome I and II, Arthritic pain, Sports injury pain, pain related to infection e.g. HIV, Post-chemotherapy pain, Post-stroke pain, Post-operative pain, neuralgia, conditions associated with Visceral pain such as Irritable bowel syndrome, and Angina pain; Inflammatory disorders (including Inflammatory bowel disease), Renal/urinary disorders (including urinary retention, benign prostatic hypertrophy, chronic renal failure, renal disease); Respiratory disorders (such as Chronic obstructive pulmonary disease, Asthma).

The invention further relates to compounds, compositions and medicaments for the treatment of androgen receptor mediated cancers, including, but not limited to, Prostate cancer, Liver Cancer, Colon cancer, Endometrial cancer, Pancreatic cancer and cancers associated with other organs of the body including the central nervous system and peripheral nervous system.

Compounds of the invention include compounds according to the formula (1)

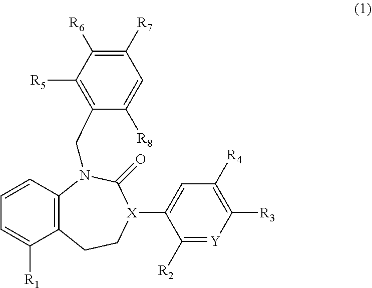

and salts thereof, wherein

### X is CH or N;

### Y is CH or N;

### R₁ is H or F;

R₂ is H, C₁-C₃ alkyl or C₁-C₃ alkoxy;
R₃ is C₁-C₃ alkyl or C₁-C₃ alkoxy;
R₄ is SO(n)CH₃ where n is 0-2 or C₁-C₃ alkyl or C₁-C₃ alkoxy where the alkyl or alkoxy groups are optionally substituted with one or more fluorine atoms;
R₅ is H, halo, cyano, C₁-C₃ alkyl or C₁-C₃ alkoxy;
R₆ is H, halo, cyano, C₁-C₃ alkyl or C₁-C₃ alkoxy;
R₇ is H, halo, cyano, C₁-C₃ alkyl or C₁-C₃ alkoxy; wherein the C₁-C₃ alkyl group can be substituted with C₁-C₃ alkoxy; and

### R₈ is H or F.

Specific embodiments include the following substituents, which can be combined in any particular combination without limitation:

X can be CH. X can be N.

Y can be CH. Y can be N.

R₁ can be H. R₁ can be F.

R₂ can be H. R₂ can be C₁-C₃ alkyl. R₂ can be C₁-C₃ alkoxy. R₂ can be CH₃. R₂ can be OCH₃.

R₃ can be C₁-C₃ alkyl. R₃ can be C₁-C₃ alkoxy. R₃ can be CH₃. R₃ can be OCH₃. R₃ can be CD₃. R₃ can be OCD₃.

R₄ can be SO(n)CH₃ where n is 0-2. R₄ can be SCH₃. R₄ can be S(O)CH₃. R₄ can be SO₂CH₃. R₄ can be C₁-C₃ alkyl. R₄ can be C₁-C₃ alkyl where the alkyl group is substituted with one or more fluorine atoms. R₄ can be C₁-C₃ alkoxy. R₄ can be C₁-C₃ alkoxy where the alkoxy group is substituted with one or more fluorine atoms. R₄ can be CF₂CH₃. R₄ can be OCF₂H.

R₅ can be H. R₅ can be halo. R₅ can be cyano R₅ can be C₁-C₃ alkyl. R₅ can be C₁-C₃ alkoxy. R₅ can be F. R₅ can be CH₃.

R₆ can be H. R₆ can be halo. R₆ can be cyano. R₆ can be C₁-C₃ alkyl. R₆ can be C₁-C₃ alkoxy. R₆ can be CN. R₆ can be F.

R₇ can be H. R₇ can be halo R₇ can be cyano R₇ can be C₁-C₃ alkyl. R₇ can be or C₁-C₃ alkoxy. R₇ can be C₁-C₃ alkyl substituted with C₁-C₃ alkoxy. R₇ can be F. R₇ can be Cl. R₇ can be Br. R₇ can be OCH₃. R₇ can be OCH(CH₃)₂. R₇ can be OCD₃. R₇ can be CH₂OCH₃. R₇ can be CH₂OCD₃. R₇ can be CH₂CH₂CH₃.

R₈ can be H. R₈ can be F.

Compounds of the invention also include compounds according to the formula (1a)

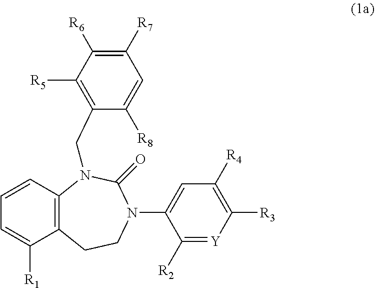

and salts thereof, wherein R₁, R₂, R₃, R₄, R₅, R₆, R₇, R₈ and Y are as defined above.

Compounds of the invention also include compounds according to the formula (1b)

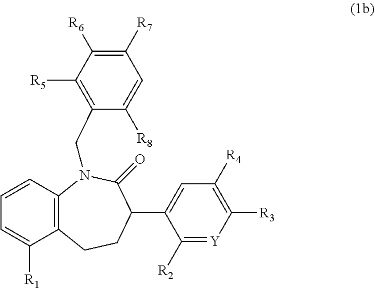

and salts thereof, wherein R₁, R₂, R₃, R₄, R₅, R₆, R₇, R₈ and Y are as defined above.

Compounds of the invention also include compounds according to the formula (1c)

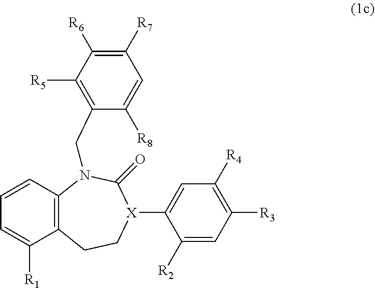

and salts thereof, wherein R₁, R₂, R₃, R₄, R₅, R₆, R₇, R₈ and X are as defined above.

Compounds of the invention also include compounds according to the formula (1d)

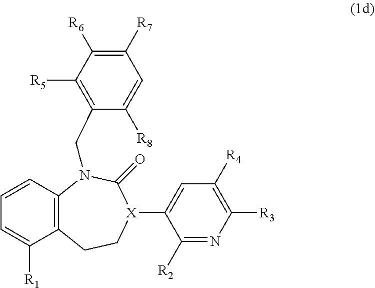

and salts thereof, wherein R₁, R₂, R₃, R₄, R₅, R₆, R₇, R₈ and X are as defined above.

Compounds of the invention also include compounds according to the formula (2a)

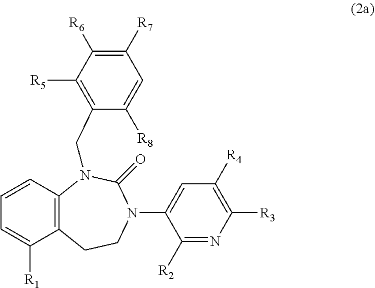

and salts thereof, wherein R₁, R₂, R₃, R₄, R₅, R₆, R₇ and R₈ are as defined above.

Compounds of the invention also include compounds according to the formula (2b)

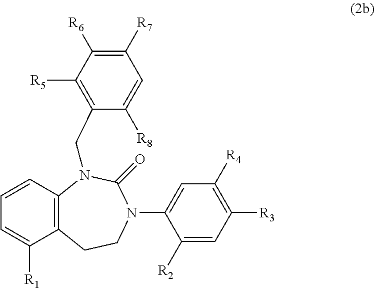

and salts thereof, wherein R₁, R₂, R₃, R₄, R₅, R₆, R₇ and R₈ are as defined above.

Compounds of the invention also include compounds according to the formula (2c)

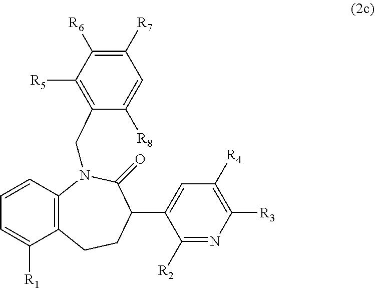

and salts thereof, wherein R₁, R₂, R₃, R₄, R₅, R₆, R₇ and R₈ are as defined above.

Compounds of the invention also include compounds according to the formula (2d)

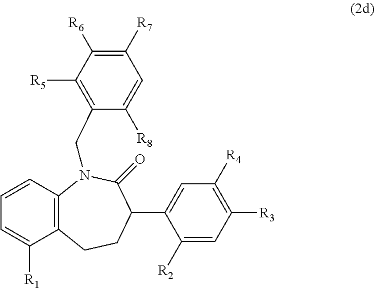

and salts thereof, wherein R₁, R₂, R₃, R₄, R₅, R₆, R₇ and R₈ are as defined above.

Specific examples of compounds include
1-(4-bromo-2,6-difluorobenzyl)-3-[3-(1,1-difluoroethyl)-4-methylphenyl]-6-fluoro-1,3,4,5-tetrahydro-2H-1,3-benzodiazepin-2-one
1-(4-bromo-2,6-difluorobenzyl)-3-{5-(1,1-difluoroethyl)-6-[(²H₃)methyloxy]pyridin-3-yl}-6-fluoro-1,3,4,5-tetrahydro-2H-1,3-benzodiazepin-2-one
1-(4-bromo-2,6-difluorobenzyl)-3-[5-(1,1-difluoroethyl)-6-methoxypyridin-3-yl]-6-fluoro-1,3,4,5-tetrahydro-2H-1,3-benzodiazepin-2-one
3-[5-(1,1-difluoroethyl)-6-methoxypyridin-3-yl]-6-fluoro-1-(2-fluoro-4-methoxybenzyl)-1,3,4,5-tetrahydro-2H-1,3-benzodiazepin-2-one
3-[5-(1,1-difluoroethyl)-6-methoxypyridin-3-yl]-1-(2,6-difluoro-4-{[(²H₃)methyloxy]methyl}benzyl)-6-fluoro-1,3,4,5-tetrahydro-2H-1,3-benzodiazepin-2-one
3-[5-(1,1-difluoroethyl)-6-methoxypyridin-3-yl]-1-{2,6-difluoro-4-[(²H₃)methyloxy]benzyl}-6-fluoro-1,3,4,5-tetrahydro-2H-1,3-benzodiazepin-2-one
3-[5-(1,1-difluoroethyl)-6-methoxypyridin-3-yl]-1-[2,6-difluoro-4-(methoxymethyl)benzyl]-6-fluoro-1,3,4,5-tetrahydro-2H-1,3-benzodiazepin-2-one
3-[5-(1,1-difluoroethyl)-6-methoxypyridin-3-yl]-1-(2,6-difluoro-4-methoxybenzyl)-6-fluoro-1,3,4,5-tetrahydro-2H-1,3-benzodiazepin-2-one
1-(4-chloro-2,6-difluorobenzyl)-3-[5-(1,1-difluoroethyl)-6-methoxypyridin-3-yl]-6-fluoro-1,3,4,5-tetrahydro-2H-1,3-benzodiazepin-2-one
1-(4-bromo-2-fluorobenzyl)-3-[5-(1,1-difluoroethyl)-6-methoxypyridin-3-yl]-6-fluoro-1,3,4,5-tetrahydro-2H-1,3-benzodiazepin-2-one
1-(4-bromo-2,6-difluorobenzyl)-3-[5-(1,1-difluoroethyl)-6-methoxypyridin-3-yl]-6-fluoro-1,3,4,5-tetrahydro-2H-1-benzazepin-2-one
(3R)-1-(4-bromo-2,6-difluorobenzyl)-3-[5-(1,1-difluoroethyl)-6-methoxypyridin-3-yl]-6-fluoro-1,3,4,5-tetrahydro-2H-1-benzazepin-2-one
3-({3-[5-(1,1-difluoroethyl)-6-methoxypyridin-3-yl]-6-fluoro-2-oxo-2,3,4,5-tetrahydro-1H-1-benzazepin-1-yl}methyl)-4-fluorobenzonitrile
3-({(3R)-3-[5-(1,1-difluoroethyl)-6-methoxypyridin-3-yl]-6-fluoro-2-oxo-2,3,4,5-tetrahydro-1H-1-benzazepin-1-yl}methyl)-4-fluorobenzonitrile
1-(4-bromo-2,6-difluorobenzyl)-6-fluoro-3-[6-methoxy-5-(methylsulfonyl)pyridin-3-yl]-1,3,4,5-tetrahydro-2H-1,3-benzodiazepin-2-one
6-fluoro-3-[6-methoxy-5-(methylsulfonyl)pyridin-3-yl]-1-(2,3,6-trifluoro-4-methoxybenzyl)-1,3,4,5-tetrahydro-2H-1,3-benzodiazepin-2-one
1-(2,6-difluoro-4-methoxybenzyl)-6-fluoro-3-[6-methoxy-5-(methylsulfonyl)pyridin-3-yl]-1,3,4,5-tetrahydro-2H-1,3-benzodiazepin-2-one
1-(4-bromo-2-fluoro-6-methylbenzyl)-6-fluoro-3-[6-methoxy-5-(methylsulfonyl)pyridin-3-yl]-1,3,4,5-tetrahydro-2H-1,3-benzodiazepin-2-one
1-(2,4-difluoro-6-methylbenzyl)-6-fluoro-3-[6-methoxy-5-(methylsulfonyl)pyridin-3-yl]-1,3,4,5-tetrahydro-2H-1,3-benzodiazepin-2-one
1-(4-bromo-2,6-difluorobenzyl)-6-fluoro-3-[6-methoxy-5-(methylsulfonyl)pyridin-3-yl]-1,3,4,5-tetrahydro-2H-1-benzazepin-2-one
(3R)-1-(4-bromo-2,6-difluorobenzyl)-6-fluoro-3-[6-methoxy-5-(methylsulfonyl)pyridin-3-yl]-1,3,4,5-tetrahydro-2H-1-benzazepin-2-one
(3S)-1-(4-bromo-2,6-difluorobenzyl)-6-fluoro-3-[6-methoxy-5-(methylsulfonyl)pyridin-3-yl]-1,3,4,5-tetrahydro-2H-1-benzazepin-2-one
1-(4-chloro-2,6-difluorobenzyl)-6-fluoro-3-[6-methoxy-5-(methylsulfonyl)pyridin-3-yl]-1,3,4,5-tetrahydro-2H-1-benzazepin-2-one
(3R)-1-(4-chloro-2,6-difluorobenzyl)-6-fluoro-3-[6-methoxy-5-(methylsulfonyl)pyridin-3-yl]-1,3,4,5-tetrahydro-2H-1-benzazepin-2-one
1-(4-bromo-2,6-difluorobenzyl)-3-[5-(1,1-difluoroethyl)-2,6-dimethoxypyridin-3-yl]-6-fluoro-1,3,4,5-tetrahydro-2H-1,3-benzodiazepin-2-one
3-[5-(1,1-difluoroethyl)-2,6-dimethoxypyridin-3-yl]-1-[2,6-difluoro-4-(methoxymethyl)benzyl]-6-fluoro-1,3,4,5-tetrahydro-2H-1,3-benzodiazepin-2-one
1-(2,6-difluoro-4-methoxybenzyl)-3-[2,6-dimethoxy-5-(methylsulfonyl)pyridin-3-yl]-6-fluoro-1,3,4,5-tetrahydro-2H-1,3-benzodiazepin-2-one
1-(4-bromo-2,6-difluorobenzyl)-6-fluoro-3-[4-methyl-3-(methylsulfanyl)phenyl]-1,3,4,5-tetrahydro-2H-1,3-benzodiazepin-2-one
1-(4-bromo-2,6-difluorobenzyl)-6-fluoro-3-[4-methyl-3-(methylsulfonyl)phenyl]-1,3,4,5-tetrahydro-2H-1,3-benzodiazepin-2-one
1-(2,6-difluoro-4-propylbenzyl)-6-fluoro-3-[4-methyl-3-(methylsulfonyl)phenyl]-1,3,4,5-tetrahydro-2H-1,3-benzodiazepin-2-one
1-(4-bromo-2,6-difluorobenzyl)-3-[2,4-dimethyl-5-(methylsulfonyl)phenyl]-1,3,4,5-tetrahydro-2H-1,3-benzodiazepin-2-one
1-[2,6-difluoro-4-(propan-2-yloxy)benzyl]-3-[2,4-dimethyl-5-(methylsulfonyl)phenyl]-1,3,4,5-tetrahydro-2H-1,3-benzodiazepin-2-one
1-(4-bromo-2,6-difluorobenzyl)-3-[2,4-dimethyl-5-(methylsulfonyl)phenyl]-6-fluoro-1,3,4,5-tetrahydro-2H-1,3-benzodiazepin-2-one
1-(4-bromo-2,6-difluorobenzyl)-3-[5-(1,1-difluoroethyl)-6-methylpyridin-3-yl]-6-fluoro-1,3,4,5-tetrahydro-2H-1,3-benzodiazepin-2-one
3-[5-(1,1-difluoroethyl)-6-methylpyridin-3-yl]-1-[2,6-difluoro-4-(methoxymethyl)benzyl]-6-fluoro-1,3,4,5-tetrahydro-2H-1,3-benzodiazepin-2-one
3-[5-(1,1-difluoroethyl)-6-methylpyridin-3-yl]-1-(2,6-difluoro-4-propylbenzyl)-6-fluoro-1,3,4,5-tetrahydro-2H-1,3-benzodiazepin-2-one
1-(4-bromo-2,6-difluorobenzyl)-3-[5-(1,1-difluoroethyl)-6-methylpyridin-3-yl]-6-fluoro-1,3,4,5-tetrahydro-2H-1-benzazepin-2-one
(3R)-1-(4-bromo-2,6-difluorobenzyl)-3-[5-(1,1-difluoroethyl)-6-methylpyridin-3-yl]-6-fluoro-1,3,4,5-tetrahydro-2H-1-benzazepin-2-one
1-(4-bromo-2,6-difluorobenzyl)-3-[5-(difluoromethoxy)-6-methylpyridin-3-yl]-6-fluoro-1,3,4,5-tetrahydro-2H-1,3-benzodiazepin-2-one
1-(4-bromo-2,6-difluorobenzyl)-6-fluoro-3-[6-methyl-5-(methylsulfonyl)pyridin-3-yl]-1,3,4,5-tetrahydro-2H-1-benzazepin-2-one
(3R)-1-(4-bromo-2,6-difluorobenzyl)-6-fluoro-3-[6-methyl-5-(methylsulfonyl)pyridin-3-yl]-1,3,4,5-tetrahydro-2H-1-benzazepin-2-one
3-[5-(1,1-difluoroethyl)-6-methylpyridin-3-yl]-1-[2,6-difluoro-4-(methoxymethyl)benzyl]-6-fluoro-1,3,4,5-tetrahydro-2H-1-benzazepin-2-one
(3R)-3-[5-(1,1-difluoroethyl)-6-methylpyridin-3-yl]-1-[2,6-difluoro-4-(methoxymethyl)benzyl]-6-fluoro-1,3,4,5-tetrahydro-2H-1-benzazepin-2-one
3-({3-[5-(1,1-difluoroethyl)-6-methylpyridin-3-yl]-6-fluoro-2-oxo-2,3,4,5-tetrahydro-1H-1-benzazepin-1-yl}methyl)-4-fluorobenzonitrile
3-({(3R)-3-[5-(1,1-difluoroethyl)-6-methylpyridin-3-yl]-6-fluoro-2-oxo-2,3,4,5-tetrahydro-1H-1-benzazepin-1-yl}methyl)-4-fluorobenzonitrile
   and salts thereof.

To the extent that any of the compounds described have chiral centres, the present invention extends to all optical isomers of such compounds, whether in the form of racemates or resolved enantiomers. The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

Pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulfuric acids; from organic acids, such as tartaric, acetic, citric, malic, lactic, fumaric, benzoic, glycolic, gluconic, succinic, arylsulfonic acids; and from metals such as sodium, magnesium, or preferably, potassium and calcium.

Also encompassed are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates.

For a more detailed discussion of solvates and the methods used to make and characterise them, see Bryn et al, Solid-State Chemistry of Drugs, Second Edition, published by SSCI, Inc of West Lafayette, Ind., USA, 1999, ISBN 0-967-06710-3. The term “pharmaceutical composition” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

The compounds of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

Therapeutic dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

The magnitude of an effective dose of a compound will, of course, vary with the nature of the severity of the condition to be treated and with the particular compound and its route of administration. The selection of appropriate dosages is within the ability of one of ordinary skill in this art, without undue burden. In general, the daily dose range may be from about 10 μg to about 30 mg per kg body weight of a human and non-human animal, preferably from about 50 μg to about 30 mg per kg of body weight of a human and non-human animal, for example from about 50 μg to about 10 mg per kg of body weight of a human and non-human animal, for example from about 100 μg to about 30 mg per kg of body weight of a human and non-human animal, for example from about 100 μg to about 10 mg per kg of body weight of a human and non-human animal and most preferably from about 100 μg to about 1 mg per kg of body weight of a human and non-human animal.

### Preparation of the Compounds of the Invention

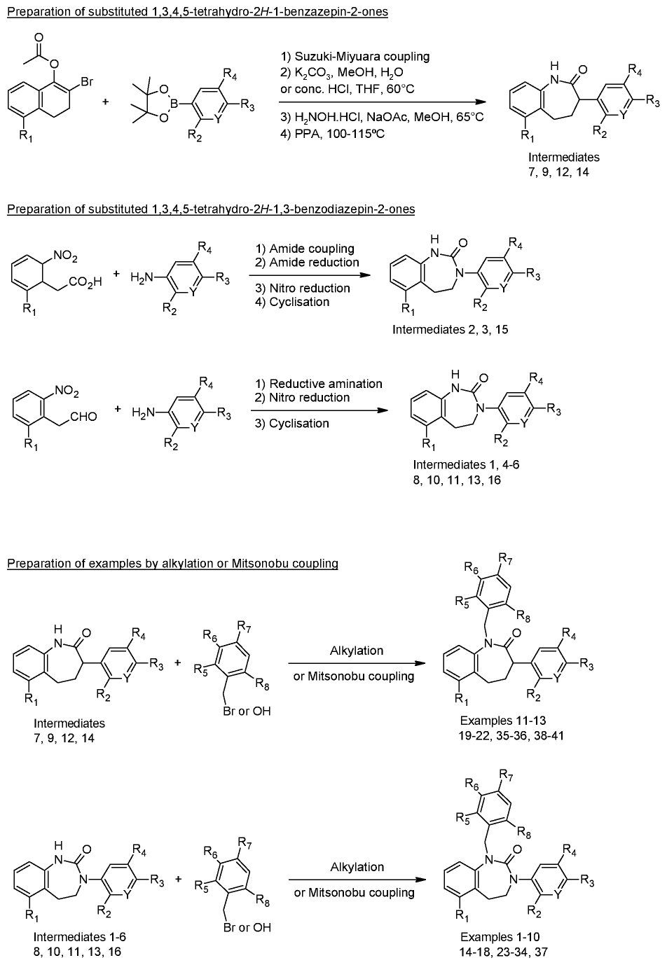

Compounds of the invention may be prepared by routes including those in FIG. 1.

Details of many of the standard transformations such as those in the routes below and others which could be used to perform the same transformations can be found in standard reference textbooks such as “Organic Synthesis”, M. B. Smith, McGraw-Hill (1994) or “Advanced Organic Chemistry”, 4^{th} edition, J. March, John Wiley & Sons (1992).

Compounds of the invention may be prepared by an alkylation reaction between a substituted 3-aryl- or 3-heteroaryl-1,3,4,5-tetrahydro-2H-1-benzazepin-2-one or substituted 3-aryl- or 3-heteroaryl-1,3,4,5-tetrahydro-2H-1,3-benzodiazepin-2-one and a substituted benzyl halide, for example a benzyl bromide. The reaction conditions will typically use a base such as potassium carbonate, sodium hydride, sodium tert-butoxide or sodium bis(trimethylsilyl)amide in a suitable solvent such as THE or DMF at a suitable temperature, for example between room temperature and 100° C. Alternatively, compounds of the invention may be prepared by reaction between a substituted 1,3,4,5-tetrahydro-2H-1-benzazepin-2-one or substituted 1,3,4,5-tetrahydro-2H-1,3-benzodiazepin-2-one and a substituted benzyl alcohol, using triphenylphosphine and an azodicarboxylate such as TMAD in a solvent such as DCM at a temperature such as room temperature. Such a reaction is commonly known as a Mitsonobu coupling (Swamy et al, Chem. Rev., 2009, 109, 2551-2561).

Exemplary reagents for alkylation or Mitsonobu coupling are shown below:

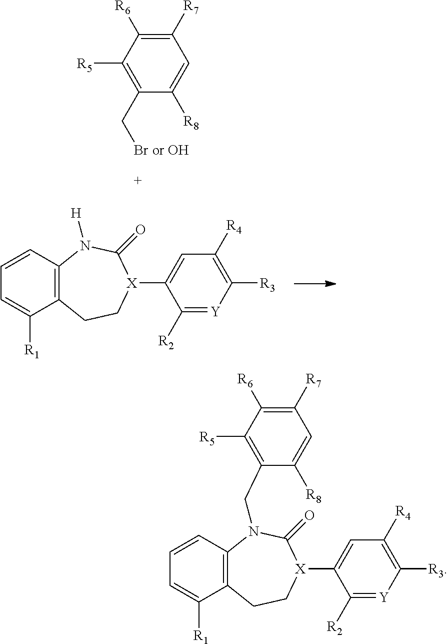

Where appropriate, compounds of the invention which contain a chiral centre or centres may be separated into their individual stereoisomers using separation techniques such as preparative chiral supercritical fluid chromatography (SFC).

Substituted 3-aryl- or 3-heteroaryl-1,3,4,5-tetrahydro-2H-1-benzazepin-2-ones may be prepared by routes including that shown in FIG. 1, where a palladium-mediated catalytic coupling (commonly known as a Suzuki-Miyaura coupling, Miyaura et al, Chem. Rev., 1995, 95, 2457-2483) between an optionally substituted 2-bromo-3,4-dihydronaphthalen-1-yl acetate and a substituted aryl or substituted heteroaryl boronic ester or boronic acid forms a substituted 2-aryl- or 2-heteroaryl-3,4-dihydronaphthalen-1-yl acetate. The palladium catalyst may be tetrakis(triphenylphosphine)palladium(0) for example, and is used in the presence of a suitable base, for example sodium carbonate, in a suitable solvent or mixture of solvents, for example THF, water, toluene or ethanol, at an elevated temperature, for example 80° C. or 90° C.

Substituted 2-aryl- or 2-heteroaryl-3,4-dihydronaphthalen-1-yl acetates can be hydrolysed to substituted 2-aryl- or 2-heteroaryl-3,4-dihydronaphthalen-1(2H)-ones under basic (for example potassium carbonate in a suitable solvent or mixture of solvents, for example MeOH/water, at a suitable temperature such as room temperature) or acidic conditions (for example concentrated hydrochloric acid in a suitable solvent such as THF, at a suitable temperature such as 60° C.).

Condensation of the ketone group in substituted 2-aryl- or 2-heteroaryl-3,4-dihydronaphthalen-1(2H)-ones with hydroxylamine, typically as the hydrochloride salt, and typically in the presence of sodium acetate using MeOH as solvent, and typically at an elevated temperature, for example 65° C., forms substituted 2-aryl- or 2-heteroaryl-3,4-dihydronaphthalen-1(2H)-imines. The imines can subsequently be ring expanded to substituted 3-aryl- or 3-heteroaryl-1,3,4,5-tetrahydro-2H-1-benzazepin-2-ones by heating in acidic conditions, for example in the presence of PPA at an elevated temperature, for example 110° C. or 115° C., for a suitable period of time, for example between 7 and 60 min.

Substituted 3-aryl- or 3-heteroaryl-1,3,4,5-tetrahydro-2H-1,3-benzodiazepin-2-ones may be prepared by routes including those shown in FIG. 1, where in one procedure an amide coupling between an optionally substituted (2-nitrophenyl)acetic acid and a substituted aniline or substituted aminopyridine forms a substituted (nitrophenyl)acetamide, under conditions which typically use a coupling agent such as T₃P or TBTU in the presence of a base such as DIPEA, in a suitable solvent such as THE or DMF, typically at room temperature. Subsequent reduction of the amide bond, typically using borane-THF complex in a solvent such as THE at room temperature, forms a substituted (nitrophenyl)ethanamine. Reduction of the nitro group can be achieved under standard conditions, for example using iron powder in the presence of ammonium chloride in a solvent such as EtOH-water, at a temperature between room temperature and reflux, iron powder in acetic acid at room temperature, or by hydrogenation using a suitable catalyst, for example Raney-nickel, in a solvent such as methanol at a suitable temperature, for example 50° C. The order of the two reduction steps can be reversed. The resulting substituted aniline can be cyclised to form substituted 3-aryl- or 3-heteroaryl-1,3,4,5-tetrahydro-2H-1,3-benzodiazepin-2-ones by treatment with a coupling agent such as CDI, a base such as Et₃N or DIPEA, in a suitable solvent, for example MeCN or DMF, at a suitable temperature, for example between 0° C. and 100° C.

In a second procedure for the formation of substituted 3-aryl- or 3-heteroaryl-1,3,4,5-tetrahydro-2H-1,3-benzodiazepin-2-ones, an optionally substituted (2-nitrophenyl)acetaldehyde can be reacted with a substituted aniline or substituted aminopyridine under reductive amination conditions. Reductive aminations are typically performed using a reducing agent, for example sodium cyanoborohydride, in the presence of an additive such as acetic acid or zinc cyanide, in a suitable solvent such as MeOH, at a temperature typically between room temperature and 60° C. The resulting substituted (nitrophenyl)ethanamines can be transformed in two steps to substituted 3-aryl- or 3-heteroaryl-1,3,4,5-tetrahydro-2H-1,3-benzodiazepin-2-ones using conditions such as those detailed above for nitro reduction and cyclisation.

Optionally substituted 2-bromo-3,4-dihydronaphthalen-1-yl acetates, substituted aryl or heteroaryl boronic esters or boronic acids, substituted benzyl halides, substituted benzyl alcohols and other intermediates can be formed using a number of standard transformations which will be known to those skilled in the art. Where such intermediates are commercially available they are identified by their chemical abstracts service (CAS) reference number in Table 1, where not commercially available the synthesis of the intermediates using standard transformations is detailed herein. Commercial reagents were utilized without further purification.

### General Procedures

Room temperature (rt) refers to approximately 20-27° C. ¹H NMR spectra were typically recorded at 300, 400 or 500 MHz on Bruker, Varian or JEOL instruments at ambient temperature unless otherwise specified. Chemical shift values are expressed in parts per million (ppm), i.e. (δ)-values. Standard abbreviations, or their combinations, are used for the multiplicity of the NMR signals, for example: s=singlet, br=broad, d=doublet, t=triplet, q=quartet, quin=quintet, h=heptet, dd=doublet of doublets, dt=doublet of triplets, m=multiplet. Coupling constants are listed as J values, measured in Hz. NMR and mass spectroscopy results were corrected to account for background peaks. Chromatography refers to column chromatography performed using silica and executed under positive pressure (flash chromatography) conditions.

### LCMS Methods

LCMS experiments were carried out using electrospray conditions under the conditions below (Solvents: A1=0.1% HCO₂H in water; A2=Water; A3=10 mM aqueous ammonium bicarbonate pH10; B1=MeCN; B2=0.1% HCO₂H in MeCN; B3=MeOH; C1=2.5 L H₂O+2.5 mL 28% ammonia in water solution; D1=2.5 L MeCN+135 mL H₂O+2.5 mL 28% ammonia in water solution; D2=MeCN+0.1% ammonia). LCMS data are given in the format: Mass ion, electrospray mode (positive or negative), retention time (experimental text and Table 1); Mass ion, electrospray mode (positive or negative), retention time, approximate purity (Table 2).

Method 1. Instruments: Hewlett Packard 1100 with G1315A DAD, Micromass ZQ; Column: Waters X-Bridge C-18, 2.5 micron, 2.1×20 mm or Phenomenex Gemini-NX C-18, 3 micron, 2.0×30 mm; Gradient [time (min)/solvent D1 in C1(%)]: 0.00/2, 0.10/2, 8.40/95, 10.00/95; Injection volume 1 μL; UV detection 230 to 400 nM; column temperature 45° C.; Flow rate 1.5 mL/min.

Method 2. Instruments: Waters Acquity H class UPLC with diode array detector and QDa mass detector; Column: Acquity UPLC BEH C18, 1.7 micron, 2.1×50 mm; Gradient [time (min)/solvent D2 in A2(%)]:0.00/2, 0.20/2, 2.50/98, 3.00/98; UV detection 210 to 350 nM; column temperature 50° C.; Flow rate 1.0 mL/min.

Method 3. Instruments: Waters Acquity UPLC with Acquity photodiode array detector and QDa mass detector; Column: Waters X-Select UPLC C-18, 1.7 micron, 2.1×30 mm; Gradient [time (min)/solvent B2 in A1(%)]:0.00/5, 0.11/5, 2.15/95, 2.56/95, 2.83/5, 3.00/5; Injection volume 3 μL; UV detection 210 to 400 nM; column temperature 40° C.; Flow rate 0.77 mL/min.

Method 4. Instruments: Waters Acquity UPLC; Column: Acquity UPLC BEH C18, 1.7 micron, 2.1×50 mm; Gradient [time (min)/solvent B2 in A1(%)]: 0.00/3, 0.20/3, 2.50/98, 3.80/98, 4.20/3, 4.50/3; Injection volume 0.5 μL; UV detection 215 nM; Column temperature 35° C.; Flow rate 0.6 mL/min.

Method 5. Instruments: Waters Acquity UPLC; Column: Acquity UPLC BEH C18, 1.7 micron, 2.1×50 mm; Gradient [time (min)/solvent B2 in A1(%)]: 0.00/2, 0.20/2, 1.50/98, 2.60/98, 2.61/2, 3.20/2; Injection volume 0.5 μL; UV detection 215 nM; Column temperature 45° C.; Flow rate 0.8 mL/min.

Method 6. Instruments: Agilent Technologies 1260 LC with Chemstation software, Diode Array Detector, Agilent 6130 Quadrupole MS with APCI and ES Source; Column: Phenomenex Gemini C-18, 5 micron, 4.6×50 mm; Gradient [time (min)/solvent B1 in A1(%)]:0.00/5, 1.37/98, 1.60/98, 1.83/5, 2.29/5; Injection volume 1 μL; UV detection 175-275 nM; column temperature 40° C.; Flow rate 1.2 mL/min.

Method 7. Instruments: Waters 2690 HPLC with 996 Photodiode Array Detector and Acquity QDA mass detector; Column: Waters X-Bridge C-18, 5 micron, 4.6×100 mm; Gradient [time (min)/solvent B3 in A1(%)]: 0.00/10, 3.00/10, 6.00/100, 7.00/100, 7.01/10, 10.00/10; Injection volume 10 μL; UV detection 230 nM; Flow rate 1 mL/min.

Method 8. Instruments: Waters Acquity H Class UPLC with MassLynx software, Diode Array Detector, Waters Acquity QDA Mass Detector; Column: Waters X-Bridge BEH C-18, 2.5 micron, 2.1×50 mm; Gradient [time (min)/solvent B1 in A2(%)]: 0.00/2, 4.00/98, 4.80/98; Injection volume 0.5 μL; UV detection 210 to 350 nM; column temperature 40° C.; Flow rate 0.8 mL/min.

Method 9. Instruments: Shimadzu LCMS-2010EV with single quadrupole MW, with Shimadzu SPD-M20A photodiode array and LC SOLUTIONS software; Column: Zodiac C18, 3 micron, 4.6×50 mm; Gradient [time (min)/solvent B1 in A1(%)]:0.00/30, 3.00/90, 6.00/90, 6.10/30; Injection volume 1 μL; UV detection 254 nM; column temperature 40° C.; Flow rate 0.8 mL/min.

Method 10. Instruments: Agilent Technologies 1260 Infinity LC with Chemstation software, Diode Array Detector, Agilent 6120B Single Quadrupole MS with API-ES Source; Column: Phenomenex Gemini-NX C-18, 3 micron, 2.0×30 mm; Gradient [time (min)/solvent D1 in C1(%)]:0.00/5, 2.00/95, 2.50/95, 2.60/5, 3.00/5; Injection volume 0.5 μL; UV detection 190 to 400 nM; column temperature 40° C.; Flow rate 1.5 mL/min.

Method 11. Instruments: Waters Acquity UPLC with Acquity photodiode array detector and QDa mass detector; Column: Waters Acquity CSH C-18, 1.7 micron, 2.1×30 mm; Gradient [time (min)/solvent B2 in A1(%)]:0.00/5, 9.52/95, 9.93/95, 10.20/5; Injection volume 3 μL; UV detection 210 to 400 nM; column temperature 40° C.; Flow rate 0.77 mL/min.

Method 12. Instruments: Shimadzu LCMS-2010EV with single quadrupole MW, with Shimadzu SPD-M20A photodiode array and LC SOLUTIONS software; Column: Zodiac C18, 3 micron, 4.6×50 mm; Gradient [time (min)/solvent B1 in A1(%)]:0.00/5, 3.00/50, 5.00/95, 7.00/95, 7.10/5; Injection volume 1 μL; UV detection 254 nM; column temperature 40° C.; Flow rate 0.6 mL/min.

### Preparative Chiral SFC Methods and Analytical Chiral SFC Methods

### 1. Preparative Chiral SFC Methods

| | | | | | Injection | |
|---|---|---|---|---|---|---|
| Example | Method | Instrument | Column | Mobile Phase | Volume | Flow rate |
| 20, 21 | A | 1 | Lux C4, 5 micron, | 50:50 MeOH:CO₂ | 1.00 mL | 50 mL/min |
| | | | 21.2 × 250 mm; | (0.1% v/v NH₃) | | |
| | | | Phenomenex Inc. | | | |
| 36 | B | 1 | Lux C1, 5 micron, | 20:80 MeOH:CO₂ | 0.30 mL | 50 mL/min |
| | | | 21.2 × 250 mm; | | | |
| | | | Phenomenex Inc. | | | |
| 12 | C | 1 | Lux A1, 5 micron, | 20:80 MeOH:CO₂ | 1.00 mL | 50 mL/min |
| | | | 21.2 × 250 mm; | (0.1% v/v NH₃) | | |
| | | | Phenomenex Inc. | | | |
| 13 | D | 2 | Lux A1, 5 micron, | 25:75 MeOH:CO₂ | 1.00 mL | 50 mL/min |
| | | | 21.2 × 250 mm; | (0.2% v/v NH₃) | | |
| | | | Phenomenex Inc. | | | |
| 22 | E | 2 | Lux A1, 5 micron, | 40:60 MeOH:CO₂ | 0.35 mL | 50 mL/min |
| | | | 21.2 × 250 mm; | (0.2% v/v NH₃) | | |
| | | | Phenomenex Inc. | | | |
| 39 | F | 2 | Lux C1, 5 micron, | 35:65 EtOH:CO₂ | 0.50 mL | 50 mL/min |
| | | | 21.2 × 250 mm; | (0.2% v/v NH₃) | | |
| | | | Phenomenex Inc. | | | |
| 40 | G | 1 | Chiralart SA, | 25.75 MeOH:CO₂ | 0.50 mL | 50 mL/min |
| | | | 5 micron, | | | |
| | | | 20 × 250 mm; | | | |
| | | | YMC Co. Ltd. | | | |
| 41 | H | 1 | Lux C4, 5 micron, | 20:80 MeOH:CO₂ | 0.30 mL | 50 mL/min |
| | | | 21.2 × 250 mm; | | | |
| | | | Phenomenex Inc. | | | |

Instrument 1: Sepiatec Prep SFC 100 with Prep SFC 100 control software and UV/Vis detector; Instrument 2: Berger Multigram II with SFC Pronto Version 1.5 software and Knauer UV detector; Mobile phases are all isocratic, see table above; UV detection in each method is at 210 nM; column temperature in each case is 40° C.; Back pressure regulation 125 Bar for methods A-C, 100 Bar for methods D-H.

### 2. Analytical Chiral SFC Methods

| Example | Method | Column | Mobile Phase |
|---|---|---|---|
| 20, 21 | I | Lux C4, 5 micron, 4.6 × 250 mm; | 35:65 MeOH:CO₂ (0.1% v/v NH₃) |
| | | Phenomenex Inc. | |
| 36 | J | Lux C1, 5 micron, 4.6 × 250 mm; | 20:80 MeOH:CO₂ |
| | | Phenomenex Inc. | |
| 12 | K | Lux A1, 5 micron, 4.6 × 250 mm; | 20:80 MeOH:CO₂ |
| | | Phenomenex Inc. | |
| 13 | L | Chiralart Amylose-C, 5 micron, | 20:80 MeOH:CO₂ (0.2% v/v NH₃) |
| | | 4.6 × 250 mm; YMC Co. Ltd. | |
| 22 | M | Chiralart Amylose-C, 5 micron, | 40:60 MeOH:CO₂ (0.2% v/v NH₃) |
| | | 4.6 × 250 mm; YMC Co. Ltd. | |
| 39 | N | Lux C1, 5 micron, 4.6 × 250 mm; | 30:70 EtOH:CO₂ (0.2% v/v NH₃) |
| | | Phenomenex Inc. | |
| 40 | O | Chiralart Amylose-SA, 5 micron, | 20:80 MeOH:CO₂ (0.2% v/v NH₃) |
| | | 4.6 × 150 mm; YMC Co. Ltd. | |
| 41 | P | Lux C4, 5 micron, 4.6 × 250 mm; | 20:80 MeOH:CO₂ (0.2% v/v NH₃) |
| | | Phenomenex Inc. | |

Instrument: Waters Acquity UPC² with Empower software, PDA detector and QDa mass detector; Column: see table above; Mobile phases are all isocratic, see table above, 125 Bar back pressure regulation; Injection volume 1 μL; UV detection 210-400 nM; column temperature 40° C.; Flow rate 4 mL/min.

Chiral purity data are given in the format: retention time, approximate purity (Table 2).

### ABBREVIATIONS

BAST=bis(2-methoxyethyl)aminosulfur trifluoride
CDI=1,1′-carbonyldiimidazole
DCE=1,2-dichloroethane
DCM=dichloromethane
DIBAL=diisobutylaluminum hydride

### DIPEA=N,N-diisopropylethylamine

### DMAC=N,N-dimethylacetamide

### DMAP=4-(dimethylamino)pyridine

### DMF=N,N-dimethylformamide

DMP=Dess-Martin periodinane
DMSO=dimethylsulfoxide
EDCI=N-(3-dimethylaminopropyl)-N′-ethylcarbodiimide hydrochloride
ES=electrospray
EtOAc=ethyl acetate
h=hour(s)
IPA=isopropyl alcohol
L=litre
LC=liquid chromatography
LCMS=liquid chromatography mass spectrometry
MeCN=acetonitrile
MeOH=methanol
min=minute(s)
MS=mass spectrometry
NaHMDS=sodium bis(trimethylsilyl)amide

### NBS=N-bromosuccinimide

### NIS=N-iodosuccinimide

NMR=nuclear magnetic resonance
PPA=polyphosphoric acid
ref=relative centrifugal force
rpm=revolutions per minute
rt=room temperature
s=second(s)
SFC=supercritical fluid chromatography
TBME=tert-butyl methyl ether
TBTU=O-(benzotriazol-1-yl)-N,N,N′,N′-tetramethyluronium tetrafluoroborate
THF=tetrahydrofuran
TMAD=1,1′-azobis(N,N-dimethylformamide)
T₃P=1-propanephosphonic anhydride

Prefixes n-, s-, i-, t- and tert—have their usual meanings: normal, secondary, iso, and tertiary.

### Synthesis of Intermediates

### Preparation of Substituted 1,3,4,5-tetrahydro-2H-1-benzazepin-2-ones and 1,3,4,5-tetrahydro-2H-1,3-benzodiazepin-2-ones

### Typical Procedure for the Preparation of Substituted 1,3,4,5-tetrahydro-2H-1-benzazepin-2-ones, as Exemplified by the Preparation of Intermediate 14, 6-fluoro-3-[6-methoxy-5-(methylsulfonyl)pyridin-3-yl]-1,3,4,5-tetrahydro-2H-1-benzazepin-2-one

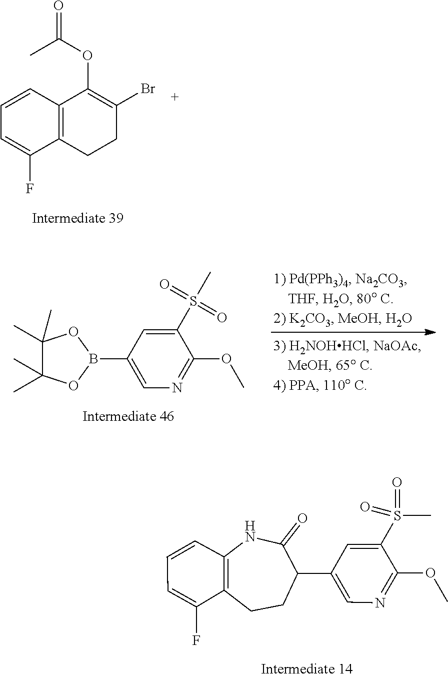

Step 1) A mixture of 2-bromo-5-fluoro-3,4-dihydronaphthalen-1-yl acetate (Intermediate 39, 2.10 g, 7.36 mmol), 2-[6-methoxy-5-(methylsulfonyl)pyridin-3-yl]-4,4,5,5-tetramethyl-1,3,2-dioxaborolane (Intermediate 46, 3.04 g, approximately 8.83 mmol, approximately 90% purity), tetrakis(triphenylphosphine)palladium(0) (425 mg, 0.37 mmol) and Na₂CO₃ (1.40 g, 13.3 mmol) in oxygen free THF (67 mL) and water (6.7 mL) was stirred at 80° C. under N₂ for 4 h. After cooling to rt, the reaction mixture was added to water (1 L) and extracted with EtOAc (500 mL then 2×300 mL). The combined organic phases were dried (Na₂SO₄), concentrated in vacuo and purified by gradient flash chromatography eluting with 10-40% EtOAc in petroleum ether to yield 5-fluoro-2-(6-methoxy-5-(methylsulfonyl)pyridin-3-yl)-3,4-dihydronaphthalen-1-yl acetate (2.14 g, 5.47 mmol) as a yellow solid.

LCMS (Method 2): m/z 392.1 (ES+), at 1.89 min.

¹H NMR: (500 MHz, CDCl₃) δ: 2.23 (s, 3H), 2.81 (t, J=8.1, 2H), 3.05 (t, J=8.1, 2H), 3.25 (s, 3H), 4.16 (s, 3H), 6.93 (d, J=7.8, 1H), 6.99 (t, J=8.6, 1H), 7.14-7.23 (m, 1H), 8.36 (d, J=2.5, 1H), 8.47 (d, J=2.4, 1H).

Step 2) Water (128 mL) and K₂CO₃ (638 mg, 4.62 mmol) were added to a solution of 5-fluoro-2-(6-methoxy-5-(methylsulfonyl)pyridin-3-yl)-3,4-dihydronaphthalen-1-yl acetate (1.60 g, 4.09 mmol) in MeOH (384 mL), the reaction flask was flushed with nitrogen and the mixture stirred vigorously at rt for 2.5 h. EtOAc (900 mL) and water (600 mL) were added, the phases were separated and the aqueous layer extracted with EtOAc (2×300 mL). The combined organic phases were washed with brine, dried (Na₂SO₄), and concentrated in vacuo to give a yellow oil which solidified on standing. Purification by column chromatography eluting with 30-50% EtOAc in petroleum ether yielded 5-fluoro-2-[6-methoxy-5-(methylsulfonyl)-3-pyridyl]-3,4-dihydro-2H-naphthalen-1-one (1.23 g, 3.52 mmol) as a white solid.

LCMS (Method 2): m/z 350.1 (ES+), at 1.72 min.

Step 3) 5-Fluoro-2-[6-methoxy-5-(methylsulfonyl)-3-pyridyl]-3,4-dihydro-2H-naphthalen-1-one (1.22 g, 3.49 mmol) was dissolved in MeOH (60 mL), hydroxylamine hydrochloride (1.21 g, 17.4 mmol) and NaOAc (1.43 g, 17.4 mmol) were added and the reaction mixture was stirred under N₂ at 65° C. for 16 h. After cooling, water (60 mL) and EtOAc (60 mL) were added, the phases were separated and the aqueous phase was extracted with EtOAc (2×60 mL). The combined organic layers were dried (Na₂SO₄) and concentrated in vacuo to yield 5-fluoro-2-[6-methoxy-5-(methylsulfonyl)-3-pyridyl]-3,4-dihydro-2H-naphthalen-1-one oxime (1.18 g, 3.24 mmol) as a white solid which was used in the next step without further purification.

LCMS (Method 2): m/z 365.1 (ES+), at 1.73 min.

Step 4) A mixture of 5-fluoro-2-[6-methoxy-5-(methylsulfonyl)-3-pyridyl]-3,4-dihydro-2H-naphthalen-1-one oxime (1.06 g, 2.91 mmol) and PPA (4.4 mL) under N₂ in a reaction flask was placed in an oil bath (pre-heated to 110° C.) for 10 min. The reaction mixture was cooled, partitioned between water (225 mL) and EtOAc (225 mL), the phases were separated and the aqueous phase was extracted with EtOAc (2×140 mL). The combined organic phases were dried (Na₂SO₄), concentrated in vacuo and purification by gradient flash chromatography eluting with 30-75% EtOAc in hexane yielded the title compound (Intermediate 14, 0.52 g, 1.43 mmol) as a white solid.

### Data in Table 1

### Intermediate 9, 6-fluoro-3-[6-methyl-5-(methylsulfonyl)pyridin-3-yl]-1,3,4,5-tetrahydro-2H-1-benzazepin-2-one

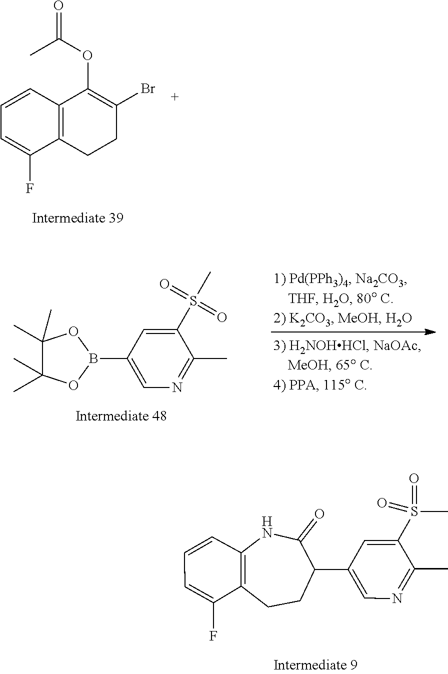

The title compound (Intermediate 9, 1.06 g, 3.04 mmol) was prepared in four steps from 2-bromo-5-fluoro-3,4-dihydronaphthalen-1-yl acetate (Intermediate 39, 6.30 g, 22.1 mmol), Pd(PPh₃)₄ (1.28 g, 1.11 mmol), 2-methyl-3-(methylsulfonyl)-5-(4,4,5,5-tetramethyl-1,3,2-dioxaborolan-2-yl)pyridine (Intermediate 48, 8.40 g, approximately 86% purity, approximately 24.3 mmol) and Na₂CO₃ (4.19 g, 39.5 mmol) in oxygen free THF (200 mL) and water (20 mL) at 80° C. for 4 h; K₂CO₃ (2.37 g, 17.1 mmol) in MeOH/water (3:1, 1.9 L) at rt for 1 h; hydroxylamine hydrochloride (5.00 g, 72.0 mmol) and NaOAc (5.91 g, 72.0 mmol) in MeOH (252 mL) at 65° C. for 16 h; and PPA (20 mL) at 115° C. for 7 min, using the methods of Intermediate Core 14, steps 1 to 4.

### Data in Table 1

### Intermediate 7, 3-[5-(1,1-difluoroethyl)-6-methylpyridin-3-yl]-6-fluoro-1,3,4,5-tetrahydro-2H-1-benzazepin-2-one

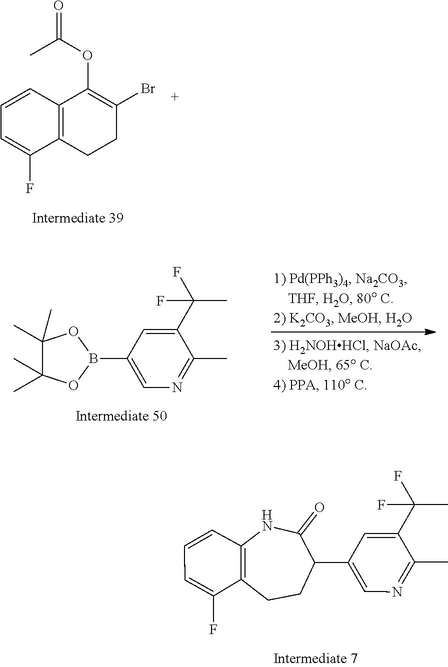 [SMILES: CC(=O)OC1=C(Br)CCc2c(F)cccc21 ; Cc1ncc(B2OC(C)(C)C(C)(C)O2)cc1C(C)(F)F ; Cc1ncc(C2CCc3c(F)cccc3NC2=O)cc1C(C)(F)F]

The title compound (Intermediate 7, 930 mg, 2.78 mmol) was prepared in four steps from 2-bromo-5-fluoro-3,4-dihydronaphthalen-1-yl acetate (Intermediate 39, 5.99 g, 21.0 mmol), Pd(PPh₃)₄ (1.21 g, 1.05 mmol), 3-(1,1-difluoroethyl)-2-methyl-5-(4,4,5,5-tetramethyl-1,3,2-dioxaborolan-2-yl)pyridine (Intermediate 50, 6.54 g, approximately 82% purity, approximately 18.9 mmol) and Na₂CO₃ (3.99 g, 37.6 mmol) in oxygen free THF (190 mL) and water (19 mL) at 80° C. for 4 h; K₂CO₃ (1.00 g, 7.24 mmol) in MeOH/water (3:1, 798 mL) at rt for 30 min; hydroxylamine hydrochloride (1.90 g, 27.3 mmol) and NaOAc (2.25 g, 27.4 mmol) in MeOH (96 mL) at 65° C. for 24 h; and PPA (22 mL) at 110° C. for 7 min, using the methods of Intermediate 14, steps 1 to 4.

### Data in Table 1

### Intermediate 12, 3-[5-(1,1-difluoroethyl)-6-methoxypyridin-3-yl]-6-fluoro-1,3,4,5-tetrahydro-2H-1-benzazepin-2-one

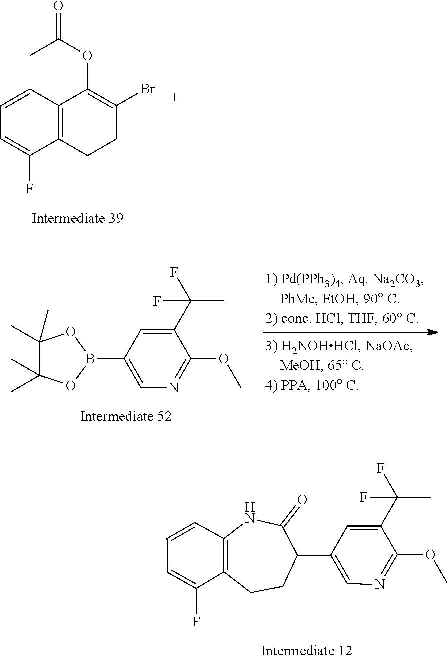

Step 1) 2-[5-(1,1-Difluoroethyl)-6-methoxypyridin-3-yl]-5-fluoro-3,4-dihydronaphthalen-1-yl acetate (2.65 g, 7.02 mmol) was prepared from 2-bromo-5-fluoro-3,4-dihydronaphthalen-1-yl acetate (Intermediate 39, 2.40 g, 8.42 mmol), 3-(1,1-difluoroethyl)-2-methoxy-5-(4,4,5,5-tetramethyl-1,3,2-dioxaborolan-2-yl)pyridine (Intermediate 52, 3.24 g, 10.8 mmol), 10% aqueous sodium carbonate solution (13.0 mL, 8.08 mmol) and tetrakis(triphenylphosphine)palladium(0) (467 mg, 0.40 mmol) in PhMe/EtOH (2:1, 60 mL) at 90° C. using the methods of Intermediate 14.

LCMS (Method 3): m/z 378 (ES+), at 1.82 min.

¹H NMR: (400 MHz, DMSO-d₆) δ: 2.01 (t, J=19.2, 3H), 2.20 (s, 3H), 2.83 (dd, J=6.8, 9.0, 2H), 2.94 (t, J=8.0, 2H), 3.97 (s, 3H), 7.05 (dd, J=1.1, 7.7, 1H), 7.14 (ddd, J=1.1, 8.3, 9.4, 1H), 7.27 (td, J=5.8, 8.0, 1H), 7.88 (d, J=2.3, 1H), 8.36 (d, J=2.3, 1H).

Step 2) 2-(5-(1,1-Difluoroethyl)-6-methoxypyridin-3-yl)-5-fluoro-3,4-dihydronaphthalen-1-yl acetate (2.65 g, 7.02 mmol) was dissolved in 5M aqueous HCl (30 mL) and heated at 60° C. for 3 h. THF (20 mL) was added and after heating at 60° C. for 3 h the reaction mixture was cooled to rt, concentrated HCl (2 mL) was added, and the reaction was stirred at rt for 72 h then at 60° C. for 3 h. The reaction was cooled to rt and carefully basified with saturated aqueous NaHCO₃ solution and extracted with EtOAc (2×200 mL). The combined organic phases were dried (MgSO₄), filtered and concentrated in vacuo. Purification by gradient flash column chromatography, eluting with 0-50% EtOAc in isohexane yielded 2-(5-(1,1-difluoroethyl)-6-methoxypyridin-3-yl)-5-fluoro-3,4-dihydronaphthalen-1(2H)-one (492 mg, 1.47 mmol) as an off-white, waxy, solid.

LCMS (Method 3): m/z 336 (ES+), at 1.68 min.

¹H NMR: (400 MHz, DMSO-d₆) δ: 2.00 (t, J=19.1, 3H), 2.29 (dtd, J=3.0, 4.7, 12.6, 1H), 2.90-3.09 (m, 1H), 3.08-3.18 (m, 1H), 3.98 (s, 3H), 4.14 (dd, J=4.3, 13.7, 1H), 7.36-7.56 (m, 2H), 7.71-7.81 (m, 2H), 8.15 (d, J=2.2, 1H). One proton obscured by the solvent peak.

Step 3) 2-[5-(1,1-Difluoroethyl)-6-methoxypyridin-3-yl]-5-fluoro-N-hydroxy-3,4-dihydronaphthalen-1(2H)-imine (450 mg, 1.28 mmol) was prepared from 2-(5-(1,1-difluoroethyl)-6-methoxypyridin-3-yl)-5-fluoro-3,4-dihydronaphthalen-1(2H)-one (472 mg, 1.41 mmol), hydroxylamine hydrochloride (440 mg, 6.33 mmol) and sodium acetate (520 mg, 6.34 mmol) in MeOH (30 mL) at 65° C. using the methods of Intermediate 14.

LCMS (Method 3): m/z 351 (ES+), at 1.65 min.

¹H NMR: (400 MHz, DMSO-d₆) δ: 1.96 (t, J=19.3, 3H), 2.07 (ddt, J=4.6, 9.0, 13.5, 1H), 2.17 (dt, J=3.9, 13.7, 1H), 2.24-2.38 (m, 1H), 2.78 (d, J=17.0, 1H), 3.89 (s, 3H), 4.73 (t, J=4.2, 1H), 7.19 (ddd, J=1.2, 8.1, 9.4, 1H), 7.31 (td, J=5.9, 8.1, 1H), 7.59 (d, J=2.4, 1H), 7.86 (dd, J=1.2, 8.0, 1H), 7.97 (dd, J=0.8, 2.4, 1H), 11.52 (s, 1H).

Step 4) The title compound (Intermediate 12, 120 mg, 0.34 mmol) was prepared from PPA (5 mL) and 2-[5-(1,1-difluoroethyl)-6-methoxypyridin-3-yl]-5-fluoro-N-hydroxy-3,4-dihydronaphthalen-1(2H)-imine (450 mg, 1.28 mmol) at 100° C. for 1 h using the methods of Intermediate 14.

### Data in Table 1

### Typical Procedure 1 for the Preparation of Substituted 1,3,4,5-tetrahydro-2H-1,3-benzodiazepin-2-ones, as Exemplified by the Preparation of Intermediate 3, 6-fluoro-3-[4-methyl-3-(methylsulfonyl)phenyl]-1,3,4,5-tetrahydro-2H-1,3-benzodiazepin-2-one

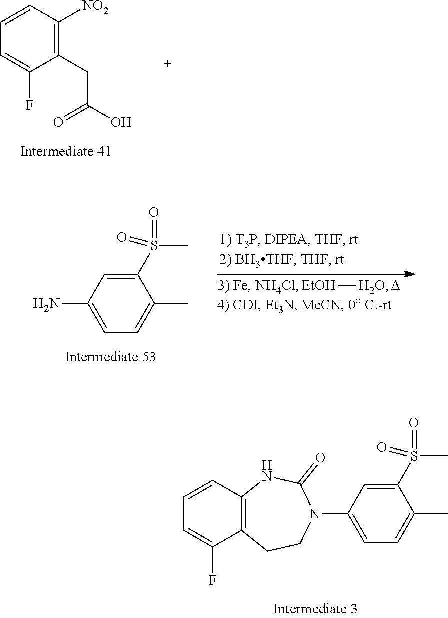

Step 1) DIPEA (0.78 mL, 4.48 mmol) and T₃P (50% solution in EtOAc, 1.78 mL, 2.99 mmol) were added to a solution of (2-fluoro-6-nitrophenyl)acetic acid (Intermediate 41, 0.30 g, 1.51 mmol) and 4-methyl-3-(methylsulfonyl)aniline (Intermediate 53, 0.27 g, 1.46 mmol) in THF (10 mL). After stirring at for rt for 16 h the reaction mixture was quenched with ice water (20 mL), extracted with EtOAc (2×15 mL), dried (Na₂SO₄) and concentrated in vacuo. Purification by trituration with n-pentane (10 mL) yielded 2-(2-fluoro-6-nitrophenyl)-N-[4-methyl-3-(methylsulfonyl)phenyl]acetamide (0.35 g, 0.96 mmol) as an off white solid.

¹H NMR: (400 MHz, DMSO-d₆) δ 2.57 (s, 3H), 3.19 (s, 3H), 4.07-4.12 (m, 2H), 7.39 (d, J=8.4, 1H), 7.56-7.87 (m, 3H), 7.95 (dt, J=1.2, 8.2, 1H), 8.19 (d, J=2.4, 1H), 10.63 (s, 1H).

Step 2) BH₃.THF (1M in THF, 5.0 mL, 5.0 mmol) was added to a stirred solution of 2-(2-fluoro-6-nitrophenyl)-N-[4-methyl-3-(methylsulfonyl)phenyl]acetamide (0.34 g, 0.93 mmol) in THF (10 mL) at 0° C. and the mixture was stirred at rt for 16 h before quenching with ice cold water (20 mL), and extracted with EtOAc (2×20 mL). The combined organic layers were dried (Na₂SO₄), concentrated in vacuo, and triturated with pentane (10 mL) to yield crude N-[2-(2-fluoro-6-nitrophenyl)ethyl]-4-methyl-3-(methylsulfonyl)aniline (0.22 g) as an off white solid.

LCMS (Method 4): m/z 353.1 (ES+), at 2.80 min.

Step 3) NH₄Cl (0.16 g, 2.99 mmol) and iron powder (0.16 g, 2.86 mmol) were added to a solution of crude N-[2-(2-fluoro-6-nitrophenyl)ethyl]-4-methyl-3-(methylsulfonyl)aniline (0.22 g) in EtOH/water (6:1, 14 mL) and the mixture was heated at reflux for 3 h. The reaction mixture was cooled to rt and filtered through a celite pad, washing the residue with ethanol (20 mL). After concentration of the filtrate in vacuo, water (20 mL) was added, and the aqueous phase was extracted with EtOAc (3×30 mL). The combined organic phases were washed with brine (30 mL), dried (Na₂SO₄), and concentrated in vacuo to yield crude N-[2-(2-amino-6-fluorophenyl)ethyl]-4-methyl-3-(methylsulfonyl)aniline (0.14 g) as a pale yellow solid.

LCMS (Method 4): m/z 323.1 (ES+), at 2.65 min.

Step 4) Et₃N (0.20 mL, 1.43 mmol) was added to a solution of crude N-[2-(2-amino-6-fluorophenyl)ethyl]-4-methyl-3-(methylsulfonyl)aniline (0.14 g) in MeCN (10 mL) at rt. After cooling to 0° C., CDI (0.19 g, 1.17 mmol) was added portion wise and the reaction mixture was stirred at rt for 16 h. Water (10 mL) was added to the reaction mixture, the resulting solid was filtered, washed with water (20 mL) and dried in vacuo to yield the title compound (90 mg, 0.26 mmol) as an off white solid.

### Data in Table 1

### Intermediate 2, 6-fluoro-3-[4-methyl-3-(methylsulfanyl)phenyl]-1,3,4,5-tetrahydro-2H-1,3-benzodiazepin-2-one

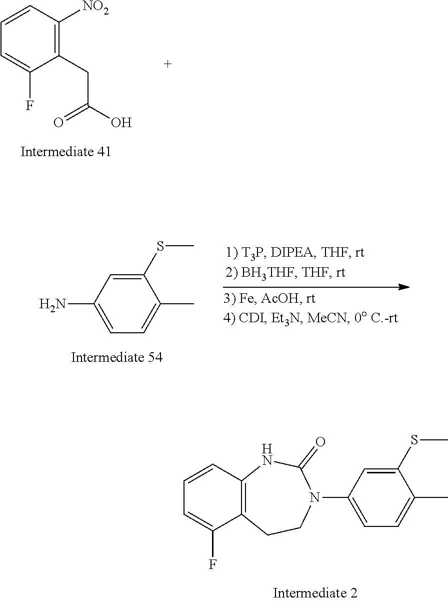

Steps 1) and 2) N-[2-(2-Fluoro-6-nitrophenyl)ethyl]-4-methyl-3-(methylsulfanyl)aniline (340 mg, 1.06 mmol) was prepared over 2 steps from (2-fluoro-6-nitrophenyl)acetic acid (Intermediate 41, 0.65 g, 3.26 mmol), 4-methyl-3-(methylsulfanyl)aniline (Intermediate 54, 0.50 g, 3.26 mmol), T₃P (1.50 g, 4.71 mmol) and DIPEA (1.75 mL, 10.1 mmol) in THF (20 mL) at rt for 16 h; and BH₃.THF (1M in THF, 3.9 mL, 3.9 mmol) in THF (10 mL) at rt for 16 h, using the methods of Intermediate 3, steps 1) and 2).

LCMS (Method 5): m/z 321.1 (ES+), at 2.15 min.

Step 3) Iron powder (5.37 mmol) was added to a solution of N-[2-(2-fluoro-6-nitrophenyl)ethyl]-4-methyl-3-(methylsulfanyl)aniline (340 mg, 1.06 mmol) in acetic acid (10 mL). After stirring at rt for 12 h the reaction mixture was filtered through a celite pad, washing the residue with EtOAc (20 mL). Water (30 mL) was added to the filtrate and the phases were separated. The aqueous phase was extracted with EtOAc (3×30 mL), the combined organic phases were washed with saturated aqueous NaHCO₃ (30 mL), dried (Na₂SO₄), and concentrated in vacuo to yield N-[2-(2-amino-6-fluorophenyl)ethyl]-4-methyl-3-(methylsulfanyl)aniline (0.30 g, 1.03 mmol) which was used for the next step without further purification.

LCMS (Method 5): m/z 291.1 (ES+), at 2.07 min.

Step 4) The title compound (Intermediate 2, 0.21 g, 0.66 mmol) was prepared from N-[2-(2-amino-6-fluorophenyl)ethyl]-4-methyl-3-(methylsulfanyl)aniline (0.30 g, 1.03 mmol), Et₃N (0.70 mL, 4.99 mmol) and CDI (0.48 g, 2.96 mmol) in MeCN (20 mL) at rt for 16 h, using the methods of Intermediate 3, step 4).

### Data in Table 1

### Intermediate 15, 3-[5-(1,1-difluoroethyl)-2,6-dimethoxypyridin-3-yl]-6-fluoro-1,3,4,5-tetrahydro-2H-1,3-benzodiazepin-2-one

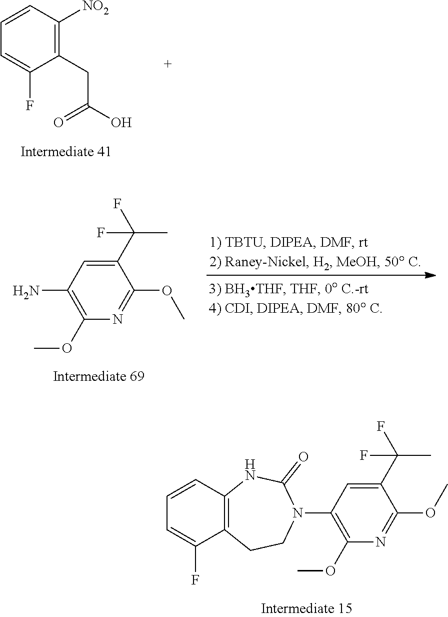 [SMILES: O=C(O)Cc1c(F)cccc1[N+](=O)[O-] ; COc1nc(OC)c(C(C)(F)F)cc1N ; COc1nc(OC)c(C(C)(F)F)cc1N1CCc2c(F)cccc2NC1=O]

Step 1) A mixture of TBTU (2.90 g, 9.03 mmol), (2-fluoro-6-nitrophenyl)acetic acid (Intermediate 41, 1.50 g, 7.53 mmol), 5-(1,1-difluoroethyl)-2,6-dimethoxypyridin-3-amine (Intermediate 69, 1.64 g, 7.52 mmol) and DIPEA (2.58 mL, 14.8 mmol) in DMF (60 mL) was stirred at rt for 2 h. The reaction mixture was then poured into water (100 mL) and stirred for 14 min, before the resulting precipitate was isolated by filtration, washed with water (100 mL) and diethyl ether (100 mL) to yield an initial crop of N-[5-(1,1-difluoroethyl)-2,6-dimethoxypyridin-3-yl]-2-(2-fluoro-6-nitrophenyl)acetamide (2.04 g). The filtrate was extracted with EtOAc (2×50 mL) and concentrated in vacuo to an oil, from which a second crop of material (0.50 g) was isolated by filtration after addition of water (20 mL). Purification of the combined crops by flash column chromatography, eluting with 40% EtOAc in hexane, followed by further purification by flash column chromatography, eluting with DCM, yielded N-[5-(1,1-difluoroethyl)-2,6-dimethoxypyridin-3-yl]-2-(2-fluoro-6-nitrophenyl)acetamide (2.10 g, 5.26 mmol).

LCMS (Method 6): m/z not observed, at 2.08 min.

¹H NMR: (400 MHz, CDCl₃) δ: 1.93 (t, J=18.5, 3H), 3.97 (s, 3H), 4.03 (s, 3H), 4.15 (d, J=1.5, 2H), 7.38-7.53 (m, 2H), 7.65 (br s, 1H), 7.93 (dt, J=1.4, 7.9, 1H), 8.63 (s, 1H).

Step 2) Raney Nickel (50% slurry in water, approximately 50-100 mg) and N-[5-(1,1-difluoroethyl)-2,6-dimethoxypyridin-3-yl]-2-(2-fluoro-6-nitrophenyl)acetamide (1.20 g, 3.01 mmol) were suspended in MeOH (60 mL) and the reaction mixture was heated in an autoclave reactor at 50° C. under 50 bar hydrogen gas pressure for 3.5 h. Further Raney Nickel (50% slurry in water, approximately 50 mg) was added, the mixture was heated at 50° C. under 50 bar hydrogen gas pressure for 90 min, then at rt overnight, followed by 55° C. for 2 h. After cooling to rt the reaction mixture was filtered through celite, rinsing with MeOH, then DCM/MeOH (1:1), and the filtrate was concentrated in vacuo to yield 2-(2-amino-6-fluorophenyl)-N-[5-(1,1-difluoroethyl)-2,6-dimethoxypyridin-3-yl]acetamide (1.0 g).

LCMS (Method 6): m/z 370.0 (ES+), at 1.75 min.

¹H NMR: (400 MHz, CDCl₃) δ: 1.94 (t, J=18.5, 3H), 3.66 (d, J=1.7, 2H), 3.95 (s, 3H), 3.99 (s, 3H), 4.44 (br s, 2H), 6.47-6.56 (m, 2H), 6.98-7.08 (m, 1H), 7.59 (s, 1H), 8.60 (s, 1H).

Step 3) 2-(2-Amino-6-fluorophenyl)-N-[5-(1,1-difluoroethyl)-2,6-dimethoxypyridin-3-yl]acetamide (0.45 g, 1.22 mmol) was suspended in THE (15 mL) and cooled to 0° C. BH₃.THF (1M in THF, 6.09 mL, 6.09 mmol) was added dropwise and the reaction allowed to warm to rt with stirring overnight. Water (20 mL) and brine (10 mL) were added and the mixture extracted with EtOAc (3×20 mL), dried (MgSO₄), and concentrated in vacuo to yield N-[2-(2-amino-6-fluorophenyl)ethyl]-5-(1,1-difluoroethyl)-2,6-dimethoxypyridin-3-amine (0.41 g, 1.15 mmol) as an orange oil which was taken into the next step without further purification due to an observation of potential instability on silica.

LCMS (Method 6): m/z 356.0 (ES+), at 1.87 min.

Step 4) DIPEA (0.40 mL, 2.30 mmol) and CDI (0.75 g, 4.63 mmol) were added to a solution of N-[2-(2-amino-6-fluorophenyl)ethyl]-5-(1,1-difluoroethyl)-2,6-dimethoxypyridin-3-amine (0.41 g, 1.15 mmol) in DMF (8 mL). After stirring at rt for 30 min and at 80° C. for 3 h the mixture was cooled to rt, water (20 mL) was added and the mixture was stirred overnight at rt. The resulting precipitate was isolated by filtration and washed with water (10 mL) and hexane (10 mL) before purification by gradient flash column chromatography eluting with 0-25% EtOAc in hexane yielded the title compound (0.14 g, 0.37 mmol) as a white solid.

### Data in Table 1

### Typical Procedure 2 for the Preparation of Substituted 1,3,4,5-tetrahydro-2H-1,3-benzodiazepin-2-ones, as Exemplified by the Preparation of Intermediate 1, 3-[3-(1,1-difluoroethyl)-4-methylphenyl]-6-fluoro-1,3,4,5-tetrahydro-2H-1,3-benzodiazepin-2-one

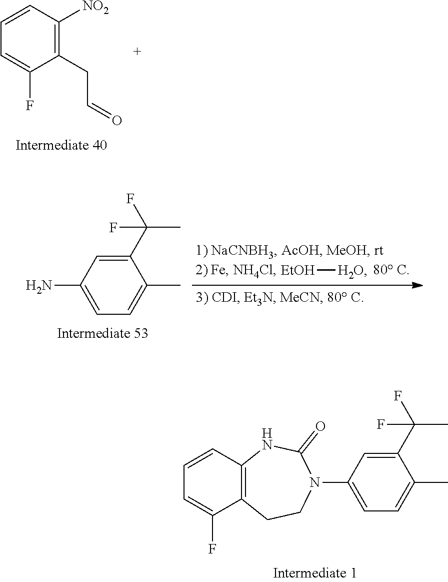

Step 1) Glacial acetic acid (0.07 g, 1.17 mmol) was added to a mixture of (2-fluoro-6-nitrophenyl)acetaldehyde (Intermediate 40, 2.20 g, 12.01 mmol), 3-(1,1-difluoroethyl)-4-methylaniline (Intermediate 53, 0.62 g, 3.62 mmol) and 4 Å molecular sieves in MeOH (15 mL) under N₂ at rt. After stirring at rt for 3 h, the reaction mixture was cooled to 0° C., sodium cyanoborohydride (2.30 g, 36.6 mmol) was added portion wise and the mixture was stirred at rt for 16 h. After concentration in vacuo and partitioning between saturated aqueous NaHCO₃ (70 mL) and EtOAc (50 mL) the aqueous phase was extracted with EtOAc (3×30 mL). The combined organic phases were dried (Na₂SO₄), concentrated in vacuo and purified by gradient flash column chromatography eluting with 0-33% EtOAc in hexane to yield 3-(1,1-difluoroethyl)-N-(2-fluoro-6-nitrophenethyl)-4-methylaniline (0.90 g, 2.66 mmol) as a brown oil.

LCMS (Method 7): m/z 339.3 (ES+), at 4.35 min.

¹H NMR: (400 MHz, DMSO-d₆) δ: 1.90 (t, J=18.9, 3H), 2.23 (s, 3H), 3.00 (ddd, J=1.9, 5.5, 9.7, 2H), 3.24-3.34 (m, 2H), 5.99 (t, J=6.3, 1H), 6.55 (ddd, J=2.5, 8.2, 21.2, 1H), 6.70 (dd, J=2.4, 7.4, 1H), 6.98 (d, J=8.2, 1H), 7.45-7.69 (m, 2H), 7.83 (dq, J=1.5, 8.2, 1H).

Step 2) A mixture of 3-(1,1-difluoroethyl)-N-(2-fluoro-6-nitrophenethyl)-4-methylaniline (0.90 g, 2.66 mmol), ammonium chloride (1.30 g, 24.3 mmol) and iron powder (0.73 g, 13.1 mmol) in EtOH/water (7:1, 16 mL) was heated at 80° C. for 4 h.

After cooling to rt the reaction mixture was passed through celite and concentrated in vacuo before partitioning between water (50 mL) and EtOAc (50 mL). The aqueous layer was extracted with EtOAc (3×30 mL) and the combined organic phases were dried (Na₂SO₄), and concentrated in vacuo. Purification by gradient flash column chromatography eluting with 0-33% EtOAc in hexane yielded N-(2-amino-6-fluorophenethyl)-3-(1,1-difluoroethyl)-4-methylaniline (0.60 g, 1.95 mmol) as an off white solid.

LCMS (Method 7): m/z 309.2 (ES+), at 4.23 min.

¹H NMR: (400 MHz, DMSO-d₆) δ: 1.90 (d, J=18.9, 3H), 2.24 (s, 3H), 2.69-2.78 (m, 2H), 3.09 (dt, J=6.0, 8.1, 2H), 5.25 (s, 2H), 5.65 (t, J=5.8, 1H), 6.29 (dddd, J=1.1, 8.2, 9.5, 13.3, 1H), 6.38-6.50 (m, 1H), 6.55 (ddd, J=2.5, 8.2, 18.2, 1H), 6.70 (t, J=2.4, 1H), 6.80-7.01 (m, 2H).

Step 3) Et₃N (1.40 mL, 10.0 mmol) was added to a solution of N-(2-amino-6-fluorophenethyl)-3-(1,1-difluoroethyl)-4-methylaniline (0.60 g, 1.95 mmol) in MeCN (10 mL). After stirring at rt for 15 min, CDI (1.58 g, 9.74 mmol) was added and the reaction mixture stirred at 80° C. for 3 h. After cooling to rt and partitioning between water (70 mL) and EtOAc (50 mL), the aqueous phase was extracted with EtOAc (3×50 mL). The combined organic phases were dried (Na₂SO₄) and concentrated in vacuo. Purification by gradient flash column chromatography eluting with 0-55% EtOAc in hexane yielded the title compound (0.16 g, 0.48 mmol) as an off white solid.

### Data in Table 1

### Intermediate 5, 3-[2,4-dimethyl-5-(methylsulfonyl)phenyl]-6-fluoro-1,3,4,5-tetrahydro-2H-1,3-benzodiazepin-2-one

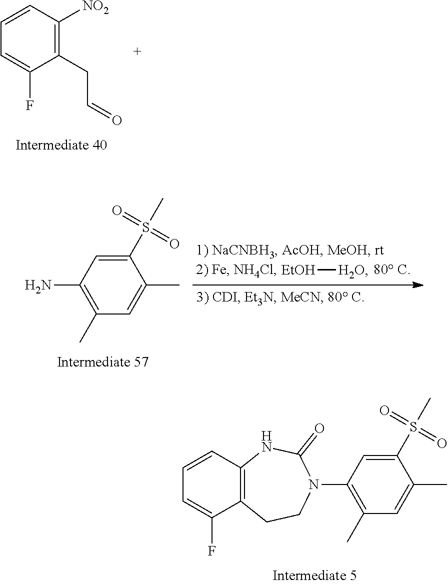

The title compound (Intermediate 5, 500 mg, 1.38 mmol) was prepared in three steps from (2-fluoro-6-nitrophenyl)acetaldehyde (Intermediate 40, 15.0 g, 81.9 mmol), 2,4-dimethyl-5-(methylsulfonyl)aniline (Intermediate 57, 3.26 g, 16.4 mmol), glacial acetic acid (0.40 g, 6.66 mmol) and sodium cyanoborohydride (15.5 g, 247 mmol) in MeOH (100 mL) at rt for 16 h; iron powder (690 mg, 12.4 mmol) and ammonium chloride (1.19 g, 22.2 mmol) in EtOH-water (7:1, 16 mL) at 80° C. for 4 h; and CDI (1.92 g, 11.8 mmol) and Et₃N (1.69 mL, 12.1 mmol) in MeCN (15 mL) at 80° C. for 3 h, using the methods of Intermediate 1, steps 1-3.

### Data in Table 1

### Intermediate 6, 3-[5-(1,1-difluoroethyl)-6-methylpyridin-3-yl]-6-fluoro-1,3,4,5-tetrahydro-2H-1,3-benzodiazepin-2-one

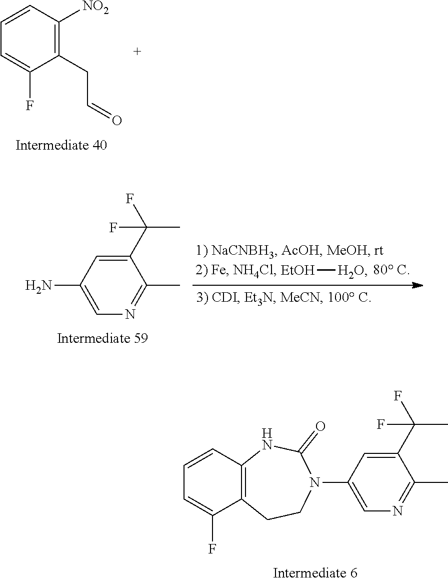

The title compound (Intermediate 6, 250 mg, 1.38 mmol) was prepared in three steps from (2-fluoro-6-nitrophenyl)acetaldehyde (Intermediate 40, 10.0 g, 54.6 mmol), 5-(1,1-difluoroethyl)-6-methylpyridin-3-amine (Intermediate 59, 1.33 g, 7.72 mmol), glacial acetic acid (0.32 g, 5.33 mmol) and sodium cyanoborohydride (10.3 g, 164 mmol) in MeOH (100 mL) at rt for 16 h; iron powder (900 mg, 16.1 mmol) and ammonium chloride (1.56 g, 29.2 mmol) in EtOH-water (7:2, 18 mL) at 80° C. for 4 h; and CDI (2.09 g, 12.9 mmol) and Et₃N (1.81 mL, 12.9 mmol) in MeCN (15 mL) at 100° C. for 30 min in a microwave reactor, using the methods of Intermediate 1, steps 1, 2 and 3.

### Data in Table 1

### Intermediate 8, 3-[5-(difluoromethoxy)-6-methylpyridin-3-yl]-6-fluoro-1,3,4,5-tetrahydro-2H-1,3-benzodiazepin-2-one

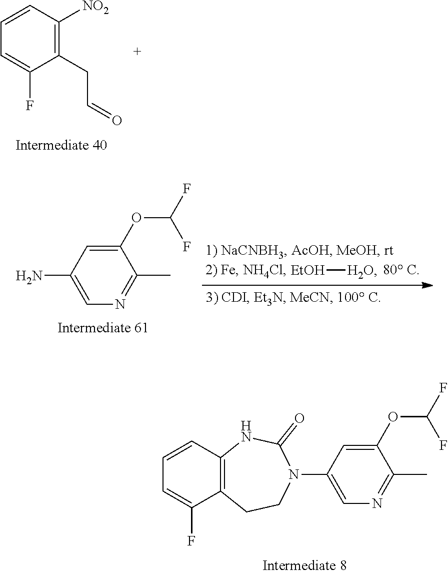

The title compound (Intermediate 8, 1.20 g, 3.64 mmol) was prepared in three steps from (2-fluoro-6-nitrophenyl)acetaldehyde (Intermediate 40, 11.0 g, 60.1 mmol), 5-(difluoromethoxy)-6-methylpyridin-3-amine (Intermediate 61, 3.00 g, 17.2 mmol), glacial acetic acid (0.36 g, 6.00 mmol) and sodium cyanoborohydride (10.8 g, 172 mmol) in MeOH (250 mL) at rt for 16 h; iron powder (3.61 g, 64.6 mmol) and ammonium chloride (6.21 g, 116 mmol) in EtOH-water (10:1, 220 mL) at 80° C. for 16 h; and CDI (7.30 g, 45.0 mmol) and Et₃N (3.80 mL, 27.1 mmol) in MeCN (100 mL) at 80° C. for 8 h, using the methods of Intermediate 1, steps 1, 2 and 3. Trituration with MeOH yielded the title compound as a yellow solid.

### Data in Table 1

### Intermediate 10, 3-{5-(1,1-difluoroethyl)-6-[(²H₃)methyloxy]pyridin-3-yl}-6-fluoro-1,3,4,5-tetrahydro-2H-1,3-benzodiazepin-2-one

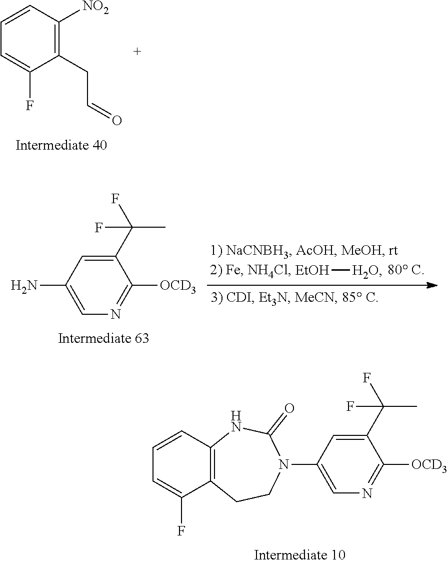

The title compound (Intermediate 10, 2.20 g, 6.21 mmol) was prepared in three steps from (2-fluoro-6-nitrophenyl)acetaldehyde (Intermediate 40, 6.48 g, 35.4 mmol), 5-(1,1-difluoroethyl)-6-[(²H₃)methyloxy]pyridin-3-amine (Intermediate 63, 4.51 g, 23.6 mmol), glacial acetic acid (0.1 mL, 1.75 mmol) and sodium cyanoborohydride (14.8 g, 236 mmol) in MeOH (90 mL) at rt for 20 h; iron powder (3.60 g, 64.5 mmol) and ammonium chloride (6.80 g, 127 mmol) in EtOH-water (2:1, 69 mL) at 80° C. for 2 h; and CDI (9.90 g, 61.1 mmol) and Et₃N (8.50 mL, 60.6 mmol) in MeCN (80 mL) at 85° C. for 8 h, using the methods of Intermediate 1, steps 1, 2 and 3. Recrystallization from MeCN yielded the title compound as a white solid.

### Data in Table 1

### Intermediate 11, 3-[5-(1,1-difluoroethyl)-6-methoxypyridin-3-yl]-6-fluoro-1,3,4,5-tetrahydro-2H-1,3-benzodiazepin-2-one

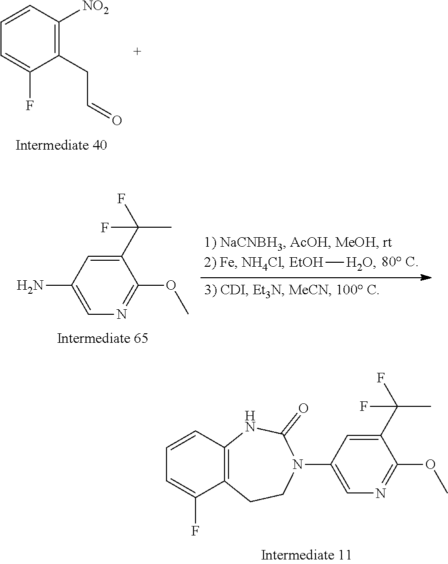

The title compound (Intermediate 11, 1.04 g, 2.96 mmol) was prepared as an off-white solid in three steps from (2-fluoro-6-nitrophenyl)acetaldehyde (Intermediate 40, 14.0 g, 76.4 mmol), 5-(1,1-difluoroethyl)-6-methoxypyridin-3-amine (Intermediate 65, 4.31 g, 22.9 mmol), glacial acetic acid (0.45 g, 7.49 mmol) and sodium cyanoborohydride (14.5 g, 231 mmol) in MeOH (100 mL) at rt for 16 h; iron powder (3.00 g, 53.7 mmol) and ammonium chloride (5.12 g, 95.7 mmol) in EtOH-water (10:3, 130 mL) at 80° C. for 6 h; and CDI (5.48 g, 33.8 mmol), Et₃N (4.74 mL, 33.8 mmol) in MeCN (40 mL) at 85° C. for 4 h, using the methods of Intermediate 1, steps 1, 2 and 3.

### Data in Table 1

### Intermediate 13, 6-fluoro-3-[6-methoxy-5-(methylsulfonyl)pyridin-3-yl]-1,3,4,5-tetrahydro-2H-1,3-benzodiazepin-2-one

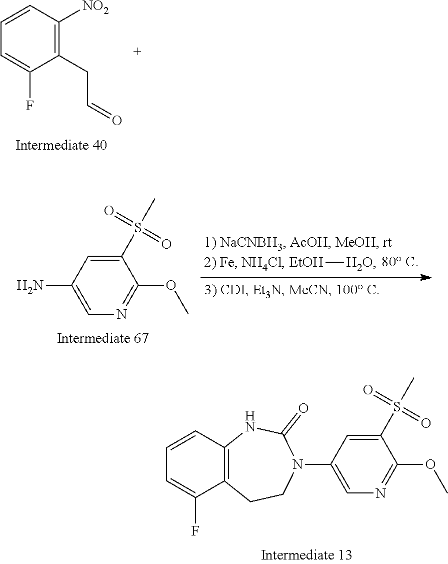

The title compound (Intermediate 13, 0.90 g, 2.46 mmol) was prepared in three steps from (2-fluoro-6-nitrophenyl)acetaldehyde (Intermediate 40, 12.0 g, 65.5 mmol), 6-methoxy-5-(methylsulfonyl)pyridin-3-amine (Intermediate 67, 3.90 g, 19.3 mmol), glacial acetic acid (0.39 g, 6.49 mmol) and sodium cyanoborohydride (12.4 g, 197 mmol) in MeOH (100 mL) at rt for 16 h; iron powder (1.74 g, 31.2 mmol) and ammonium chloride (2.98 g, 55.7 mmol) in EtOH-water (3:1, 80 mL) at 80° C. for 4 h; and CDI (3.10 g, 19.1 mmol) and Et₃N (2.70 mL, 19.3 mmol) in MeCN (40 mL) at 100° C. for 30 min in a microwave reactor (Step 3 was carried out in three batches under identical conditions which were combined before work up); using the methods of Intermediate 1, steps 1, 2 and 3. Trituration with MeOH yielded the title compound as an off-white solid.

### Data in Table 1

### Intermediate 16, 3-[2,6-dimethoxy-5-(methylsulfonyl)pyridin-3-yl]-6-fluoro-1,3,4,5-tetrahydro-2H-1,3-benzodiazepin-2-one

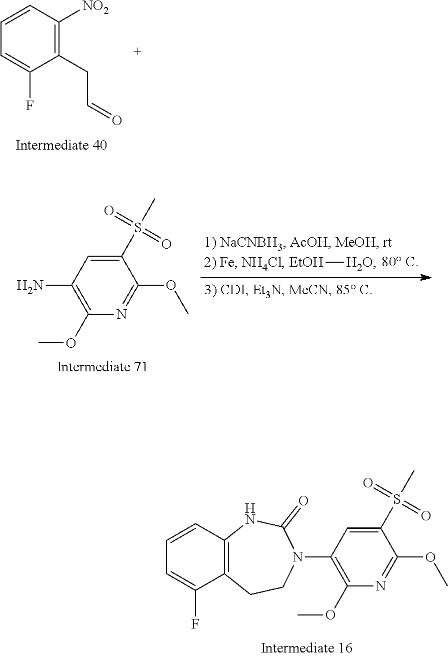

The title compound (Intermediate 16, 2.81 g, 7.11 mmol) was prepared as a cream solid in three steps from (2-fluoro-6-nitrophenyl)acetaldehyde (Intermediate 40, 2.96 g, 16.2 mmol), 2,6-dimethoxy-5-(methylsulfonyl)pyridin-3-amine (Intermediate 71, 2.50 g, 10.8 mmol), glacial acetic acid (184 μL, 3.21 mmol) and sodium cyanoborohydride (2.03 g, 32.3 mmol) in MeOH (50 mL) at rt for 3 d; iron powder (2.66 g, 47.6 mmol) and ammonium chloride (4.59 g, 85.8 mmol) in EtOH (100 mL) and water (14 mL) at 80° C. for 3.5 h; and CDI (7.33 g, 45.2 mmol) and Et₃N (6.3 mL, 44.9 mmol) in MeCN (62 mL) at 85° C. for 4 h; using the methods of Intermediate 1, steps 1, 2 and 3.

### Data in Table 1

### Intermediate 4, 3-[2,4-dimethyl-5-(methylsulfonyl)phenyl]-1,3,4,5-tetrahydro-2H-1,3-benzodiazepin-2-one

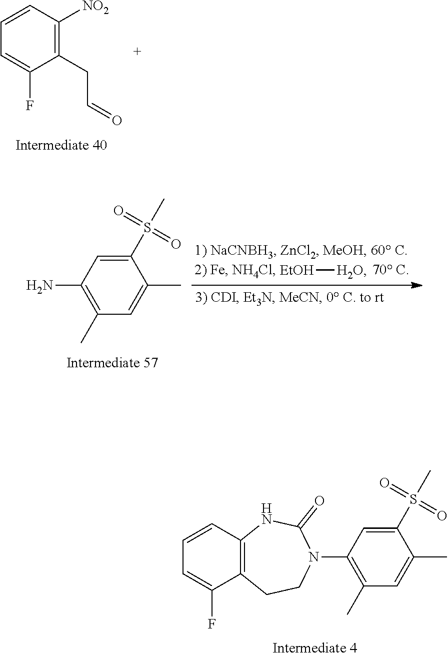

Step 1) ZnCl₂ (1.25 g, 9.17 mmol) was added to a solution of 2,4-dimethyl-5-(methylsulfonyl)aniline (Intermediate 57, 610 mg, 3.06 mmol) and (2-nitrophenyl)acetaldehyde (Intermediate 43, 510 mg, 3.09 mmol) in MeOH (15 mL) at rt and the mixture was stirred at 60° C. for 6 h. After cooling to rt, NaCNBH₃ (570 mg, 9.07 mmol) was added portion wise and the mixture was stirred at 60° C. for 16 h. The reaction mixture was concentrated in vacuo, saturated aqueous NaHCO₃ (10 mL) was added and the aqueous phase was extracted with DCM (3×10 mL). The combined organic phases were washed with brine, dried (Na₂SO₄) and concentrated in vacuo. Purification by gradient flash column chromatography eluting with 2-5% MeOH in DCM yielded 2,4-dimethyl-5-(methylsulfonyl)-N-(2-nitrophenethyl)aniline (580 mg, 1.66 mmol) as a brown solid.

MS (ESI+ve): 349.

Steps 2) and 3) The title compound (Intermediate 4, 400 mg) was prepared in two steps from 2,4-dimethyl-5-(methylsulfonyl)-N-(2-nitrophenethyl)aniline (580 mg, 1.66 mmol), iron powder (460 mg, 8.24 mmol) and ammonium chloride (795 mg, 14.9 mmol) in EtOH-water (6:1, 14 mL) at reflux for 3 h; and CDI (1.15 g, 7.09 mmol), Et₃N (0.99 mL, 7.06 mmol) in MeCN (10 mL) at rt for 16 h, using the methods of Intermediate 1, steps 2 and 3).

### Data in Table 1

### Preparation of Substituted Aryl/Heteroaryl Boronic Esters

### Intermediate 46, 2-[6-methoxy-5-(methylsulfonyl)pyridin-3-yl]-4,4,5,5-tetramethyl-1,3,2-dioxaborolane

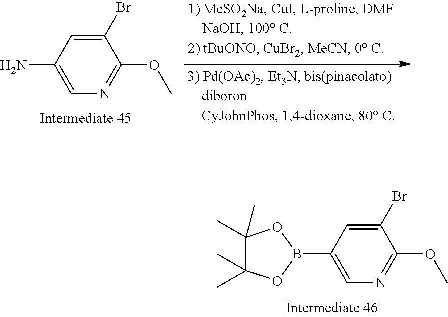

Step 1) 5-Bromo-6-methoxypyridin-3-amine (Intermediate 45, 50.0 g, 246.3 mmol), sodium methanesulfinate (50.3 g, 492.7 mmol), copper iodide (46.9 g, 246.3 mmol), L-proline (28.4 g, 246.7 mmol) and NaOH (9.85 g, 246.3 mmol) were dissolved in DMF (600 mL) and heated at 100° C. under nitrogen for 48 h. After pouring into water (2.0 L) the precipitated solid was removed by filtration and the filtrate was extracted with EtOAc (4×500 mL). The combined organic phases were dried (MgSO₄) and concentrated in vacuo. Purification by flash chromatography eluting with 50% EtOAc in petroleum ether yielded 6-methoxy-5-(methylsulfonyl)pyridin-3-amine as a brown oil (30.5 g, containing approximately 20 wt % DMF and an additional impurity) which was used in the next step without further purification.

LCMS (Method 2): m/z 203.1 (ES+), at 1.91 min.

Step 2) tert-Butyl nitrite (19.4 mL, 163.1 mmol) was added dropwise to a stirred solution of 6-methoxy-5-(methylsulfonyl)pyridin-3-amine (27.5 g) and CuBr₂ (45.6 g, 204.2 mmol) in MeCN (300 mL) under N₂ at 0° C. After stirring for 90 min at 0° C. the reaction mixture was concentrated in vacuo and the residue was partitioned between water (500 mL) and EtOAc (1 L). The organic layer was separated and washed with water (2×500 mL) and brine (500 mL), dried (MgSO₄), and concentrated in vacuo. Purification by gradient flash chromatography eluting with 20-40% EtOAc in petroleum ether yielded 5-bromo-2-methoxy-3-(methylsulfonyl)pyridine (18.9 g, 71.0 mmol) as an orange solid.

LCMS (Method 2): m/z 265.9 (ES+), at 1.51 min.

¹H NMR: (500 MHz, CDCl₃) δ: 3.23 (s, 3H), 4.12 (s, 3H), 8.35 (d, J=2.5, 1H), 8.43 (d, J=2.4, 1H).

Step 3) Pd(OAc)₂ (168 mg, 0.75 mmol) and Et₃N (7.85 mL, 56.3 mmol) were added to a stirred, oxygen free solution of 5-bromo-2-methoxy-3-(methylsulfonyl)pyridine (4.00 g, 15.0 mmol), bis(pinacolato)diboron (5.71 g, 22.5 mmol) and CyJohnPhos ((2-biphenyl)dicyclohexylphosphine) (527 mg, 1.50 mmol) in 1,4-dioxane (80 mL) under N₂. The reaction mixture was stirred at 80° C. under N₂ for 16 h before the addition of further Pd(OAc)₂ (168 mg, 0.75 mmol) and CyJohnPhos (527 mg, 1.50 mmol) at rt. After heating at 80° C. under N₂ for 16 h the reaction mixture was concentrated in vacuo and purification by flash chromatography eluting with 40% EtOAc in petroleum ether yielded the title compound (3.78 g) as a light yellow solid containing small amounts of residual bis(pinacolato)diboron.

### Data in Table 1

### Intermediate 48, 2-methyl-3-(methylsulfonyl)-5-(4,4,5,5-tetramethyl-1,3,2-dioxaborolan-2-yl)pyridine

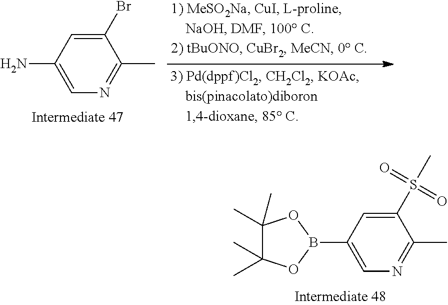

The title compound (Intermediate 48, 8.40 g, containing approximately 14% residual bis(pinacalato)diboron, 24.3 mmol) was prepared in three steps from 5-bromo-6-methylpyridin-3-amine (Intermediate 47, 26.0 g, 139.0 mmol), sodium methanesulfinate (28.4 g, 278.2 mmol), copper iodide (26.5 g, 139.1 mmol), L-proline (16.0 g, 139.0 mmol) and NaOH (5.60 g, 140.0 mmol) in DMF (300 mL) at 110° C. for 18 h; tert-butyl nitrite (11.7 mL, 98.4 mmol) and CuBr₂ (24.7 g, 110.6 mmol) in MeCN (180 mL) at 0° C. for 90 min; and [1,1′-bis(diphenylphosphino)ferrocene]dichloropalladium(II), complex with dichloromethane (1.24 g, 1.52 mmol), KOAc (8.95 g, 91.2 mmol) and bis(pinacolato)diboron (10.0 g, 39.4 mmol) in 1,4-dioxane (38 mL) at 85° C. for 18 h, using the methods of Intermediate 46, steps 1 to 3.

### Data in Table 1

### Intermediate 50, 3-(1,1-difluoroethyl)-2-methyl-5-(4,4,5,5-tetramethyl-1,3,2-dioxaborolan-2-yl)pyridine

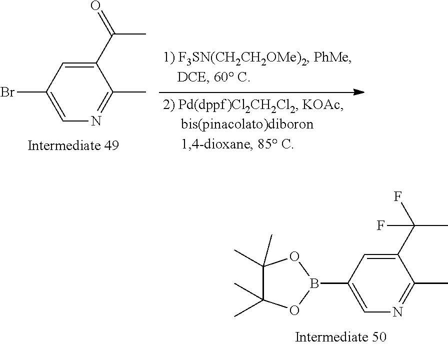 [SMILES: CC(=O)c1cc(Br)cnc1C ; Cc1ncc(B2OC(C)(C)C(C)(C)O2)cc1C(C)(F)F]

Step 1) Bis(2-methoxyethyl)aminosulfur trifluoride (50% in PhMe, 43.2 mL, 117.2 mmol) was added to a solution of 1-(5-bromo-2-methyl-3-pyridinyl)ethanone (Intermediate 49, 8.40 g, 39.2 mmol) in DCE (60 mL), the reaction mixture was stirred at 60° C. for 18 h, then cooled to rt and quenched with MeOH (25 mL). The mixture was basified with saturated aqueous NaHCO₃ to pH 8-9 and extracted with DCM (3×200 mL). The combined organic phases were dried (MgSO₄), concentrated in vacuo, and purified by gradient flash column chromatography, eluting with 0-20% EtOAc in petroleum ether, to yield 5-bromo-3-(1,1-difluoroethyl)-2-methylpyridine (5.96 g, 25.2 mmol) as a yellow oil.

LCMS (Method 2): m/z 238.0 (ES+), at 1.76 min.

¹H NMR: (500 MHz, CDCl₃) δ: 1.95 (t, J=18.3, 3H), 2.63 (t, J=2.0, 3H), 7.88 (d, J=2.2, 1H), 8.59 (d, J=2.2, 1H).

Step 2) The title compound (Intermediate 50, 8.80 g, containing approximately 18% residual bis(pinacalato)diboron) was prepared as a light yellow oil from 5-bromo-3-(1,1-difluoroethyl)-2-methylpyridine (7.07 g, 30.0 mmol), [1,1′-bis(diphenylphosphino)ferrocene]dichloropalladium(II), complex with dichloromethane (1.22 g, 1.49 mmol), KOAc (8.82 g, 89.9 mmol) and bis(pinacolato)diboron (9.89 g, 38.9 mmol) in 1,4-dioxane (46 mL) at 85° C. for 18 h, using the methods of Intermediate 46, step 3.

### Data in Table 1

### Intermediate 52, 3-(1,1-difluoroethyl)-2-methoxy-5-(4,4,5,5-tetramethyl-1,3,2-dioxaborolan-2-yl)pyridine

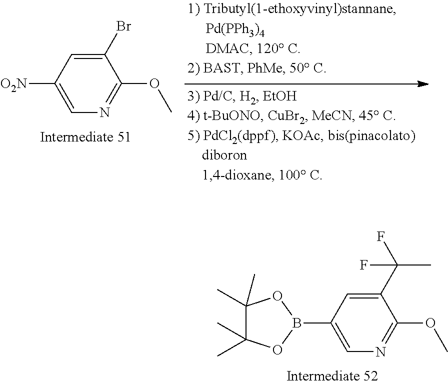

Step 1) A mixture of 3-bromo-2-methoxy-5-nitropyridine (Intermediate 51, 25 g, 107.3 mmol), tributyl(1-ethoxyvinyl)stannane (43.5 mL, 130.3 mmol) and tetrakis(triphenylphosphine)palladium(0) (8.68 g, 7.51 mmol) in anhydrous DMAC (100 mL) was purged with N₂ for 30 min and then heated at 120° C. for 3 h. After cooling to rt the mixture was poured into 1M aqueous HCl (500 mL). The mixture was stirred overnight before saturated aqueous potassium sodium tartrate solution (800 mL) was added and the mixture was extracted with EtOAc (2×300 mL). The combined organic phases were dried (MgSO₄), filtered and concentrated in vacuo. Purification by gradient flash column chromatography eluting with 0-30% EtOAc in isohexane yielded a yellow solid, which was slurried in diethyl ether and filtered to afford 1-(2-methoxy-5-nitropyridin-3-yl)ethanone (3.38 g, 17.2 mmol) as white solid.

LCMS (Method 3): m/z 197 (ES+), at 1.09 min.

¹H NMR: (400 MHz, DMSO-d₆) δ 2.62 (s, 3H), 4.13 (s, 3H), 8.66 (d, J=2.8, 1H), 9.24 (d, J=2.8, 1H).

Step 2) A solution of 1-(2-methoxy-5-nitropyridin-3-yl)ethanone (7.53 g, 38.4 mmol) and BAST (50 wt % in PhMe, 45 mL, 122 mmol) was stirred at rt overnight before heating at 50° C. for 48 h. The reaction mixture was cooled to rt and further BAST (50 wt % in PhMe, 14.2 mL, 38.4 mmol) was added. The reaction mixture was heated at 50° C. for 4 h, then stirred at rt for 2.5 d before being quenched with ice cold water and extracted with EtOAc (2×150 mL). The combined organic phases were dried (MgSO₄), filtered and concentrated in vacuo, before purification by gradient flash column chromatography eluting with 0-50% TBME in isohexane yielded 3-(1,1-difluoroethyl)-2-methoxy-5-nitropyridine (6.61 g, 30.3 mmol) as a white solid.

LCMS (Method 3): m/z not observed, at 1.40 min.

¹H NMR: (400 MHz, DMSO-d₆) δ: 2.03 (t, J=19.3, 3H), 4.10 (s, 3H), 8.46 (dt, J=0.8, 2.8, 1H), 9.21 (dt, J=0.8, 2.8, 1H).

Step 3) 3-(1,1-Difluoroethyl)-2-methoxy-5-nitropyridine (6.60 g, 30.3 mmol) was dissolved in EtOH (80 mL), 10% Pd/C (3.22 g, 3.03 mmol) was added and the mixture was hydrogenated at a pressure of 2 bar overnight. The reaction mixture was filtered through a pad of celite and concentrated in vacuo to yield 5-(1,1-difluoroethyl)-6-methoxypyridin-3-amine (5.27 g, 28.0 mmol) as a light brown semi-solid.

LCMS (Method 3): m/z 189 (ES+), at 0.79 min.

¹H NMR: (400 MHz, DMSO-d₆) δ: 1.93 (t, J=19.0, 3H), 3.79 (s, 3H), 5.05 (br s, 2H), 7.19 (d, J=2.8, 1H), 7.60 (dt, J=1.0, 2.8, 1H).

Step 4) CuBr₂ (3.66 g, 16.4 mmol) and tert-butyl nitrite (1.95 mL, 16.4 mmol) were dissolved in anhydrous MeCN (50 mL) and heated at 45° C. for 10 min. After cooling to rt 5-(1,1-difluoroethyl)-6-methoxypyridin-3-amine (1.07 g, 5.69 mmol) in MeCN (5 mL) was added dropwise to the mixture, which was then heated at 50° C. for 3 h. After cooling to rt the reaction mixture was combined with a second reaction mixture formed from CuBr₂ (15.0 g, 67.0 mmol), tert-butyl nitrite (7.96 mL, 66.9 mmol) and 5-(1,1-difluoroethyl)-6-methoxypyridin-3-amine (4.2 g, 25.3 mmol) in anhydrous MeCN (40 mL) at 50° C. for 3 h. Saturated aqueous NaHCO₃ (100 mL) was added and the mixture was extracted with EtOAc (100 mL). The organic phase was dried (MgSO₄), filtered and concentrated in vacuo. Purification by gradient flash column chromatography, eluting with 0-50% DCM in heptane yielded 5-bromo-3-(1,1-difluoroethyl)-2-methoxypyridine (2.57 g, 10.2 mmol) as an orange liquid.

LCMS (Method 3): m/z 252/254 (ES+), at 1.60 min.

¹H NMR: (400 MHz, DMSO-d₆) δ 1.98 (t, J=19.2, 3H), 3.94 (s, 3H), 7.98 (dt, J=0.8, 2.5, 1H), 8.43 (dt, J=0.9, 2.5, 1H).

Step 5) 5-Bromo-3-(1,1-difluoroethyl)-2-methoxypyridine (2.45 g, 9.72 mmol) was dissolved in anhydrous 1,4-dioxane (20 mL) and treated with KOAc (2.80 g, 28.5 mmol), bis(pinacolato)diboron (3.14 g, 12.4 mmol) and PdCl₂(dppf) (0.348 g, 0.48 mmol). The mixture was purged under N₂ for 10 min before heating at 100° C. for 4 h. The reaction mixture was filtered through celite, washed with TBME (50 mL) and DCM (50 mL). The organic washings were concentrated in vacuo, purification by gradient flash column chromatography, eluting with 0-100% EtOAc in isohexane yielded the title compound (3.37 g) as an orange semi-solid in approximately 70% purity as assessed by ¹H NMR, which was used without further purification in the synthesis of Examples 11 to 13.

### Data in Table 1

### Preparation of Substituted Anilines and Aminopyridines

### Intermediate 56, 3-(1,1-difluoroethyl)-4-methylaniline

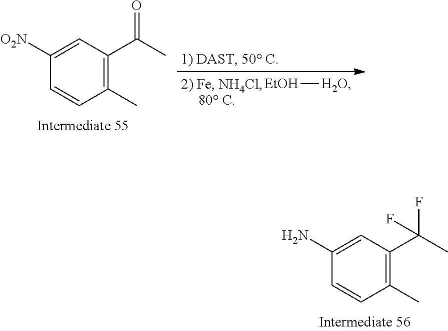

Step 1) 1-(2-Methyl-5-nitrophenyl)ethan-1-one (Intermediate 55, 1.30 g, 7.26 mmol) was dissolved in DAST (7 mL) at rt and the reaction mixture was stirred at 50° C. for 48 h. The reaction mixture was partitioned between water (40 mL) and EtOAc (30 mL), the phases were separated and the aqueous phases was extracted with EtOAc (3×30 mL). The combined organic phases were dried (Na₂SO₄) and concentrated in vacuo. Purification by gradient flash column chromatography eluting with 0-3% EtOAc in hexane yielded 2-(1,1-difluoroethyl)-1-methyl-4-nitrobenzene (1.30 g, 6.46 mmol) as a pale yellow solid.

LCMS (Method 7): m/z not observed, at 4.11 min.

¹H NMR: (400 MHz, CDCl₃) δ: 2.02 (t, J=18.4, 3H), 2.61 (s, 3H), 7.43 (d, J=8.3, 1H), 8.15-8.25 (m, 1H), 8.38 (d, J=2.5, 1H).

Step 2) A mixture of 2-(1,1-difluoroethyl)-1-methyl-4-nitrobenzene (1.30 g, 6.46 mmol), ammonium chloride (3.10 g, 58.0 mmol) and iron powder (1.82 g, 32.6 mmol) in EtOH/water (7:1, 16 mL) was heated at 80° C. for 3 h. After cooling to rt the reaction mixture was passed through celite and concentrated in vacuo before partitioning between water (50 mL) and EtOAc (50 mL). The aqueous layer was extracted with EtOAc (3×30 mL) and the combined organic phases were dried (Na₂SO₄), and concentrated in vacuo. Purification by gradient flash column chromatography eluting with 0-17% EtOAc in hexane yielded the title compound (0.60 g, 3.50 mmol) as an off white solid.

### Data in Table 1

### Intermediate 59, 5-(1,1-difluoroethyl)-6-methylpyridin-3-amine

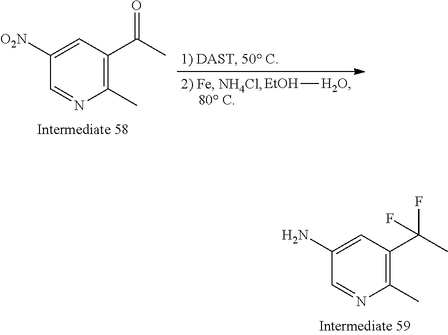

The title compound (Intermediate 59, 3.50 g, 20.3 mmol) was prepared in two steps from 1-(2-methyl-5-nitropyridin-3-yl)ethanone (Intermediate 58, 6.00 g, 33.3 mmol) and DAST (16 mL) at 50° C. for 16 h; then iron powder (6.90 g, 123.5 mmol) and ammonium chloride (11.9 g, 222 mmol) in EtOH/water (4:1, 50 mL) at 80° C. for 4 h, using the methods of Intermediate 56.

### Data in Table 1

### Intermediate 61, 5-(difluoromethoxy)-6-methylpyridin-3-amine

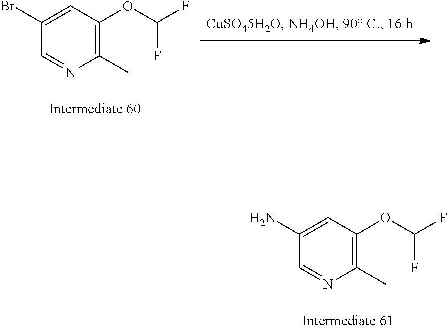

5-Bromo-3-(difluoromethoxy)-2-methylpyridine (Intermediate 60, 5.80 g, 24.4 mmol) was dissolved in 25% aqueous ammonium hydroxide solution (60 mL), CuSO₄.5H₂O (1.22 g, 4.89 mmol) was added at rt and the reaction mixture was stirred at 90° C. in a hydrogenator for 16 h. After dilution with aqueous NaOH solution (1M, 50 mL) the mixture was partitioned between water (200 mL) and EtOAc (50 mL), the aqueous phase was extracted with EtOAc (3×50 mL), the combined organic phases were dried (Na₂SO₄), and concentrated in vacuo. Purification by gradient flash column chromatography on basic alumina eluting with 0-25% EtOAc in hexane yielded the title compound (3.30 g, 18.9 mmol) as a yellow liquid.

### Data in Table 1

### Intermediate 63, 5-(1,1-difluoroethyl)-6-[(²H₃)methyloxy]pyridin-3-amine

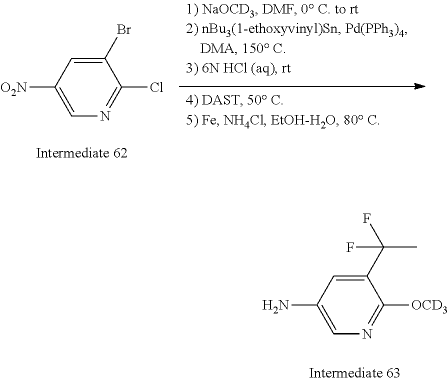

Step 1) Sodium methoxide-d₃ (1.81 g, 31.7 mmol) was added to a solution of 3-bromo-2-chloro-5-nitropyridine (Intermediate 62, 5.0 g, 21.1 mmol) in DMF (50 mL) at 0° C. The reaction mixture was stirred at rt for 1 h before adding to ice cold water (250 mL) and stirring for 30 min. The precipitated solid was isolated by filtration, washed with water (2×50 mL), and dissolved in EtOAc (150 mL). The organic phase was washed with brine (50 mL), dried (Na₂SO₄), and concentrated in vacuo to yield 3-bromo-2-[(²H₃)methyloxy]-5-nitropyridine (3.5 g, 14.8 mmol) as an off-white solid.

¹H NMR: (400 MHz, CDCl₃) δ: 8.62 (d, J=2.4, 1H), 9.02 (d, J=2.4, 1H)

Sodium methoxide-d₃ preparation: Sodium metal (1.0 g, 43.5 mmol) was added portion wise to methanol-d₃ (10 mL), keeping the internal temperature below 50° C., and stirred for 2 h. Further methanol-d₃ (5 mL) was added dropwise and after stirring for 1 h the clear solution was concentrated in vacuo to yield sodium methoxide-d₃ (2.4 g) as an off white solid.

Steps 2) and 3) Tributyl(1-ethoxyvinyl)stannane (7.2 mL, 21.6 mmol) and tetrakis(triphenylphosphine)palladium(0) (1.47 g, 1.27 mmol) were added to a solution of 3-bromo-2-[(²H₃)methyloxy]-5-nitropyridine (5.0 g, 21.2 mmol) in dry DMAC (50 mL) in a sealed tube, and heated at 130° C. for 1 h. Three reactions, each of 5 g scale, were performed simultaneously and were worked up together. The reaction mixtures were added to ice cold aqueous HCl (1N, 1.0 L) and stirred for 2 h at rt before extracting with EtOAc (3×100 mL). The combined organic phases were washed with brine (2×50 mL), dried (Na₂SO₄), and concentrated in vacuo. Purification by flash column chromatography, eluting with 5% EtOAc in petroleum ether yielded 1-{2-[(²H₃)methyloxy]-5-nitropyridin-3-yl}ethanone (7.2 g, 36.1 mmol) as an off-white solid.

¹H NMR: (400 MHz, CDCl₃) δ: 2.69 (s, 3H), 8.87 (d, J=2.8, 1H), 9.19 (d, J=2.8, 1H).

Steps 4) and 5) The title compound (Intermediate 63, 4.51 g, 24.0 mmol) was prepared in two steps from 1-{2-[(²H₃)methyloxy]-5-nitropyridin-3-yl}ethanone (7.0 g, 35.1 mmol) and DAST (14 mL) at 60° C. for 48 h; then iron powder (7.6 g, 136 mmol) and ammonium chloride (14.4 g, 269 mmol) in EtOH/water (2:1, 90 mL) at 80° C. for 2 h, using the methods of Intermediate 56.

### Data in Table 1

### Intermediate 65, 5-(1,1-difluoroethyl)-6-methoxypyridin-3-amine

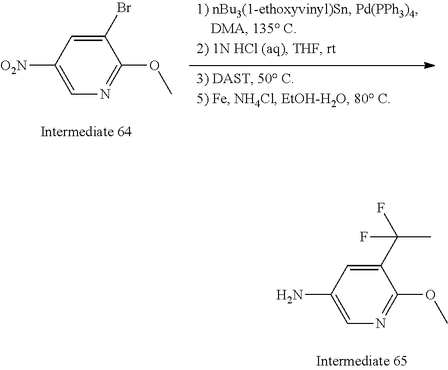

The title compound (Intermediate 65, 6.20 g, 32.9 mmol) was prepared in four steps from 3-bromo-2-methoxy-5-nitropyridine (Intermediate 64, 20.0 g total, 85.8 mmol), tributyl(1-ethoxyvinyl)stannane (31.1 g total, 86.1 mmol) and tetrakis(triphenylphosphine)palladium(0) (5.97 g total, 5.17 mmol) in DMAC (200 mL total) in a microwave reactor at 135° C. for 15 min (reactions were carried out in 10 equal scale batches under identical conditions); aqueous HCl (1M, 93.0 mL) in THF (95 mL) at rt for 2 h; DAST (27 mL) at 50° C. for 16 h; and iron powder (10.3 g, 184 mmol) and ammonium chloride (17.7 g, 331 mmol) in EtOH/water (5:2, 140 mL) at 80° C. for 4 h, using the methods of Intermediate 56.

### Data in Table 1

### Intermediate 67, 6-methoxy-5-(methylsulfonyl)pyridin-3-amine

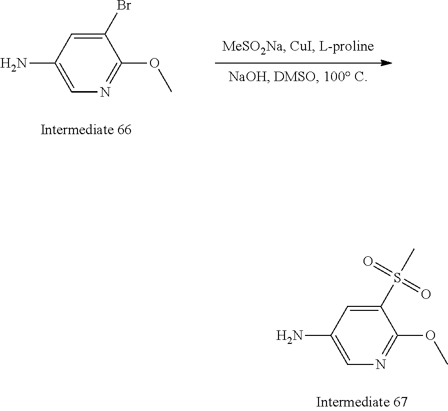

Sodium methanesulfinate (8.30 g, 81.3 mmol), copper iodide (3.06 g, 16.1 mmol), L-proline (1.84 g, 16.0 mmol) and NaOH (660 mg, 16.5 mmol) were added to a solution of 5-bromo-6-methoxypyridin-3-amine (Intermediate 66, 8.20 g, 40.4 mmol) in DMF (100 mL). After heating at 100° C. for 48 h the reaction mixture was cooled to rt, partitioned between water (200 mL) and EtOAc (150 mL), and the aqueous phase was extracted with EtOAc (3×100 mL). The combined organic phases were dried (Na₂SO₄) and concentrated in vacuo. Purification by gradient flash column chromatography eluting with 0-44% EtOAc in hexane yielded the title compound (3.90 g, 19.3 mmol) as a brown solid.

### Data in Table 1

### Intermediate 69, 5-(1,1-difluoroethyl)-2,6-dimethoxypyridin-3-amine

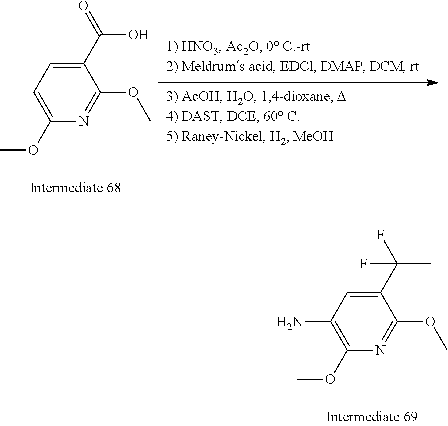

Step 1) 2,6-Dimethoxypyridine-3-carboxylic acid (Intermediate 68, 15.0 g, 81.9 mmol) was added portion wise over 5 min to a mixture of concentrated nitric acid (25 mL, 0.55 mol) and acetic anhydride (75 mL, 0.79 mol), keeping the temperature below 5° C. The resulting mixture was stirred at 0-5° C. for 3 h, then at rt for 2 h, before being carefully added to ice (approximately 1.5 kg). The resulting off-white solid was isolated by filtration and dried in vacuo at 40° C. overnight. The crude product was suspended in hexane (400 mL) and heated at reflux for 5 min, before being cooled to rt and isolated by filtration, yielding 2,6-dimethoxy-5-nitropyridine-3-carboxylic acid (16.3 g, 71.4 mmol) as an off-white solid which was used in the next step without further purification.

LCMS (Method 6): m/z 227 (ES−), at 1.33 min.

Step 2) 2,2-Dimethyl-1,3-dioxane-4,6-dione (Meldrum's acid, 9.85 g, 68.3 mmol) was added to a stirred solution of 2,6-dimethoxy-5-nitro-pyridine-3-carboxylic acid (12.0 g, 52.6 mmol) in DCM (200 mL) at rt. DMAP (12.9 g, 0.11 mol) and EDCI (20.2 g, 0.11 mol) were added and the resulting solution was stirred at rt for 18 h before concentration in vacuo. Water (200 mL) was added to the residue and the resulting suspension acidified to pH 2 with 1M aqueous HCl. The suspension was extracted with DCM (2×150 mL), the combined organic extracts were extracted with water (3×100 mL), dried (Na₂SO₄) and concentrated in vacuo to afford a yellow semi-solid. Trituration with hexane-EtOAc (1:1, 100 mL) yielded 5-[(2,6-dimethoxy-5-nitropyridin-3-yl)carbonyl]-2,2-dimethyl-1,3-dioxane-4,6-dione (17.2 g, 48.6 mmol) as a yellow-brown solid which contained residual DMAP by ¹H NMR and was used in the next step without further purification.

LCMS (Method 6): m/z 353 (ES−), at 1.50 min.

Step 3) Acetic acid (41.4 g, 0.69 mol) and water (14 mL) were added to a suspension of crude 5-[(2,6-dimethoxy-5-nitropyridin-3-yl)carbonyl]-2,2-dimethyl-1,3-dioxane-4,6-dione (24.4 g, 68.9 mmol) in 1,4-dioxane (140 mL). The mixture was heated at reflux for 2 h, cooled to rt and concentrated in vacuo. The residue was taken up in water (200 mL) and aqueous NaHCO₃ was carefully added until the phase was basic. The resulting precipitate was isolated by filtration and dried in vacuo at 40° C. to yield 1-(2,6-dimethoxy-5-nitropyridin-3-yl)ethanone (10.5 g, 46.4 mmol) as a yellow solid.

LCMS (Method 6): m/z not observed, at 1.54 min.

¹H NMR: (400 MHz, CDCl₃) δ: ppm 2.61 (s, 3H), 4.16 (s, 3H), 4.18 (s, 3H), 8.94 (s, 1H).

Step 4) DAST (61.3 mL, 464 mmol) was added carefully to a suspension of 1-(2,6-dimethoxy-5-nitro-3-pyridyl)ethanone (10.5 g, 46.4 mmol) in DCE (10 mL) at rt. The reaction mixture was stirred at 60° C. for 20 h before cooling to rt and being added in small portions to ice (approximately 2 kg). The aqueous mixture was extracted with EtOAc (2×300 mL), dried (Na₂SO₄), and concentrated in vacuo to afford a brown solid which was purified by flash column chromatography, eluting with 20% EtOAc in hexane. Further purification by trituration with hexane yielded 3-(1,1-difluoroethyl)-2,6-dimethoxy-5-nitropyridine (7.46 g, 30.1 mmol) as an off-white solid.

LCMS (Method 6): m/z not observed, at 1.71 min.

¹H NMR: (400 MHz, CDCl₃) δ: ppm 1.98 (t, J=18.8, 3H), 4.11 (s, 3H), 4.15 (s, 3H), 8.57 (s, 1H).

Step 5) Raney Nickel (slurry in water, approximately 0.75 g) was added to a solution of 3-(1,1-difluoroethyl)-2,6-dimethoxy-5-nitro-pyridine (3.2 g, 12.9 mmol) in MeOH (70 mL). The mixture was stirred in an autoclave reactor under 50 bar hydrogen gas pressure for 3 h. A moderate exotherm was observed on charging the autoclave. The crude reaction mixture was filtered through a short pad of celite and concentrated in vacuo before being taken up in hexane (150 mL). The residual brown semi-solid was washed with hexane (2×50 mL) and the combined organic extracts were dried (Na₂SO₄), and concentrated in vacuo to yield the title compound as a brown oil that solidified on standing (2.4 g, 11.0 mmol). Due to potential instability on silica the title compound was used without further purification in the synthesis of Intermediate 15.

### Data in Table 1

### Intermediate 71, 2,6-dimethoxy-5-(methylsulfonyl)pyridin-3-amine

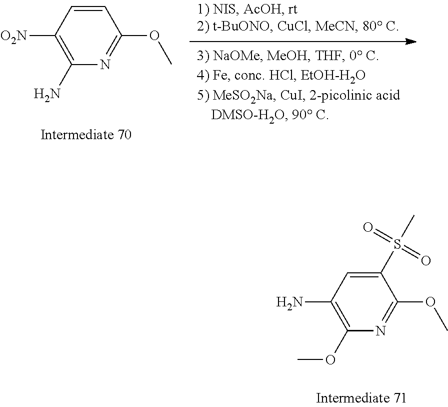

Step 1) NIS (199 g, 0.88 mol) was added to a suspension of 6-methoxy-3-nitropyridin-2-amine (Intermediate 70, 100 g, 0.59 mol) in acetic acid (1.9 L), the reaction mixture was stirred at rt for 18 h, concentrated in vacuo and partitioned between aqueous sodium hydroxide (1M, 2 L) and EtOAc (2.5 L). The phases were separated and the organic layer was washed with saturated aqueous NaHCO₃ (1.5 L), and brine (1 L), dried (Na₂SO₄), and concentrated in vacuo. The residue was triturated with heptane (1.5 L) and dried in vacuo to yield 5-iodo-6-methoxy-3-nitropyridin-2-amine (153 g, 0.52 mol) as a yellow solid.

LCMS (Method 8): m/z 293.9 (ES−), at 2.11 min.

¹H NMR: (300 MHz, DMSO-d₆) δ ppm 3.89 (s, 3H), 8.19 (br s, 2H), 8.52 (s, 1H).

Step 2) tert-Butyl nitrite (68 mL, 0.57 mol) was added to a stirred suspension of copper(I) chloride (54 g, 0.54 mol) in MeCN (1.6 L) under argon. The resulting mixture was heated to 40° C., 5-iodo-6-methoxy-3-nitropyridin-2-amine (80 g, 0.27 mol) was added portion wise and the mixture was heated at 80° C. for 2 h. After cooling to rt, EtOAc (2 L) was added, and the organic phase was washed with aqueous hydrochloric acid (2M, 2 L), brine (2×1 L), dried (Na₂SO₄), and concentrated in vacuo. Purification by flash column chromatography, eluting with heptanes yielded 2-chloro-5-iodo-6-methoxy-3-nitropyridine (32.4 g, 103 mmol) as a pale yellow solid.

LCMS (Method 8): m/z not observed, at 2.54 min.

¹H NMR: (300 MHz, DMSO-d₆) δ: ppm 3.98 (s, 3H), 8.83 (s, 1H).

Step 3) A solution of 2-chloro-5-iodo-6-methoxy-3-nitropyridine (32.4 g, 103 mmol) in THE (60 mL) was added drop wise to sodium methoxide (11.1 g, 205 mmol) in MeOH (324 mL) at 0° C. under argon. After stirring at 0° C. for 1 h additional sodium methoxide (2.80 g, 51.8 mmol) was added, and the reaction was stirred at 0° C. for 1 h before the addition of water (700 mL). The resulting solid was isolated by filtration, washed with water and dried in vacuo at 40° C. to yield 3-iodo-2,6-dimethoxy-5-nitropyridine (30.7 g, 99.0 mmol) as a pale yellow solid.

LCMS (Method 8): m/z not observed, at 2.41 min.

¹H NMR: (300 MHz, DMSO-d₆) δ: ppm 3.99 (s, 3H), 4.02 (s, 3H), 8.65 (s, 1H).

Step 4) To a suspension of 3-iodo-2,6-dimethoxy-5-nitropyridine (45.5 g, 147 mmol) in industrial methylated spirit (295 mL) was added water (46 mL), and concentrated hydrochloric acid (˜12M, 4.6 mL). Iron powder (90 g, 1.61 mol) was added portion wise and the mixture was heated at reflux for 1.5 h. After cooling to rt the mixture was filtered through a pad of celite and washed with EtOAc. The filtrate was concentrated in vacuo and purification by gradient flash column chromatography eluting with 0-10% EtOAc in heptane yielded 5-iodo-2,6-dimethoxypyridin-3-amine (33.9 g, 121 mmol) as an orange solid.

LCMS (Method 8): m/z 280.9 (ES+), at 2.10 min.

¹H NMR: (300 MHz, DMSO-d₆) δ ppm 3.75 (s, 3H), 3.84 (s, 3H), 4.45 (br s, 2H), 7.29 (s, 1H).

Step 5) 5-Iodo-2,6-dimethoxypyridin-3-amine (10 g, 35.7 mmol), sodium methanesulfinate (9.2 g, 90.1 mmol), 2-picolinic acid (0.88 g, 7.15 mmol) and copper(I) iodide (0.68 g, 3.57 mmol) were added to a mixture of DMSO (60 mL) and water (4 mL) which had been degassed with argon for 10 min. After heating at 90° C. for 4.5 h, the mixture was cooled to rt, water (150 mL) was added and the mixture was extracted with EtOAc (4×200 mL). The combined organic phases were washed with brine (150 mL), dried (Na₂SO₄), and concentrated in vacuo. The resulting dark brown solid residue was triturated with isopropanol (150 mL) and the resulting solid was isolated by filtration, washed with isopropanol (150 mL) and diethyl ether (150 mL) and dried to yield the title compound (5.25 g, 22.6 mmol) as a brown solid.

### Data in Table 1

### Preparation of Substituted Benzyl Bromides and Benzyl Alcohols

### Intermediate 18, 4-(bromomethyl)-3,5-difluorophenyl propan-2-yl ether

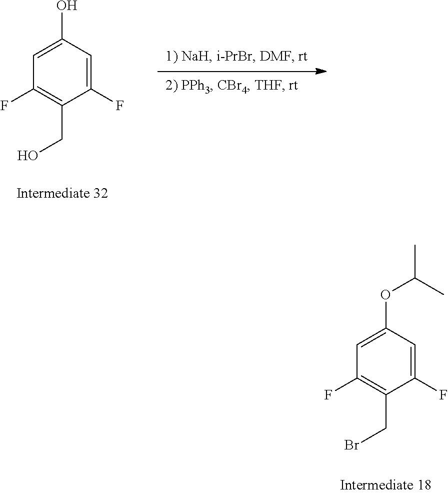

Step 1) NaH (0.53 g, approximately 55% dispersion in mineral oil, 11.8 mmol) was added to a solution of 3,5-difluoro-4-(hydroxymethyl)phenol (Intermediate 32, 1.80 g, 11.2 mmol) in DMF (18 mL) at 0° C. After stirring for 15 min at 0° C. isopropyl bromide (1.10 mL, 11.7 mmol) was added dropwise and the reaction mixture was stirred at for 16 h. Saturated aqueous NH₄Cl solution (10 mL) was added and the mixture was extracted with EtOAc (2×20 mL). The combined organic layers were washed with brine, dried (Na₂SO₄), and concentrated in vacuo. Purification by gradient flash column chromatography eluting with 20-30% EtOAc in hexane yielded (2,6-difluoro-4-isopropoxyphenyl)methanol (760 mg, 3.76 mmol) as a colorless liquid.

¹H NMR: (400 MHz, DMSO-d₆) δ ppm 1.24 (d, J=5.9, 6H), 4.39 (s, 2H), 4.61-4.67 (m, 1H), 5.07 (br s, 1H), 6.68 (d, J=10.0, 2H).

Step 2) CBr₄ (1.87 g, 5.64 mmol) was added to a mixture of (2,6-difluoro-4-isopropoxyphenyl)methanol (760 mg, 3.76 mmol) and triphenylphosphine (1.48 g, 5.64 mmol) in THF (12 mL) at 0° C. and the reaction mixture was stirred for 2 h at rt before concentration in vacuo. Purification by gradient flash column chromatography eluting with 5-10% EtOAc in hexane yielded the title compound (450 mg, 1.70 mmol) as a colorless liquid.

### Data in Table 1

### Intermediate 19, [2,6-difluoro-4-(methoxymethyl)phenyl]methanol

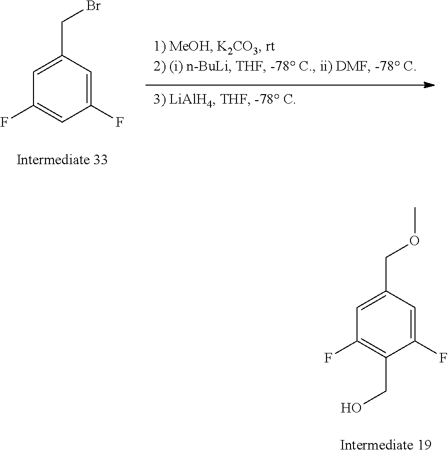

Step 1) K₂CO₃ (2.01 g, 14.5 mmol) was added to a solution of 1-(bromomethyl)-3,5-difluorobenzene (Intermediate 33, 1.00 g, 4.83 mmol) in MeOH (10 mL) under N₂ and the reaction mixture as stirred at rt for 16 h. After filtration through celite and rinsing the celite with ether (25 mL) the filtrate was concentrated in vacuo (cautiously due to product volatility) then triturated with n-pentane to yield 1,3-difluoro-5-(methoxymethyl)benzene (0.90 g, 5.69 mmol) as a colorless liquid.

¹H NMR: (400 MHz, CDCl₃) δ: ppm 3.40 (s, 3H), 4.43 (s, 2H), 6.71 (tt, J=2.3, 8.9, 1H), 6.80-6.94 (m, 2H).

Step 2) n-BuLi (1.6M in hexane, 4.20 mL, 6.72 mmol) was added to a solution of 1,3-difluoro-5-(methoxymethyl)benzene (0.90 g, 5.69 mmol) in anhydrous THF (15 mL) at −78° C. and the reaction mixture was stirred at −78° C. for 1 h. DMF (0.52 mL, 6.72 mmol) was added at −78° C. and the reaction mixture was stirred at −78° C. for 30 min before being warmed to 0° C. and partitioned between saturated aqueous NH₄Cl (50 mL) and EtOAc (50 mL). The phases were separated and the aqueous phase was extracted with EtOAc (3×30 mL). The combined organic phases were dried (Na₂SO₄), concentrated in vacuo and triturated with n-pentane to yield 2,6-difluoro-4-(methoxymethyl)benzaldehyde (0.60 g, 3.22 mmol) as a colorless liquid.

LCMS (Method 7): m/z 186.9 (ES+), at 1.71 min.

Step 3) LiAlH₄ (1M in THF, 3.90 mL, 3.90 mmol) was added to a solution of 2,6-difluoro-4-methoxymethyl)benzaldehyde (0.60 g, 3.22 mmol) in anhydrous THF (15 mL) at −78° C. and the reaction mixture was stirred at −78° C. for 30 min. After warming to 0° C. the reaction mixture was partitioned between saturated aqueous NH₄Cl (50 mL) and EtOAc (50 mL). The phases were separated and the aqueous phase was extracted with EtOAc (3×30 mL). The combined organic phases were dried (Na₂SO₄), concentrated in vacuo and purified by gradient flash column chromatography, eluting with 0-10% EtOAc in hexane, to yield the title compound (0.20 g, 1.06 mmol) as colorless liquid.

### Data in Table 1

### Intermediate 20, 4-(bromomethyl)-3,5-difluorobenzyl methyl ether

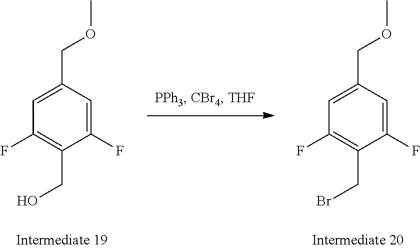

The title compound (541 mg, 2.15 mmol) was prepared as a clear oil from [2,6-difluoro-4-(methoxymethyl)phenyl]methanol (Intermediate 19, 376 mg, 2.00 mmol), CBr₄ (995 mg, 3.00 mmol) and triphenylphosphine (787 mg, 3.00 mmol) in THE (6 mL) at 0° C. for 30 min, then rt overnight, using the methods of Intermediate 18, Step 2.

### Data in Table 1

### Intermediate 21, 2-(bromomethyl)-1,3-difluoro-5-propylbenzene

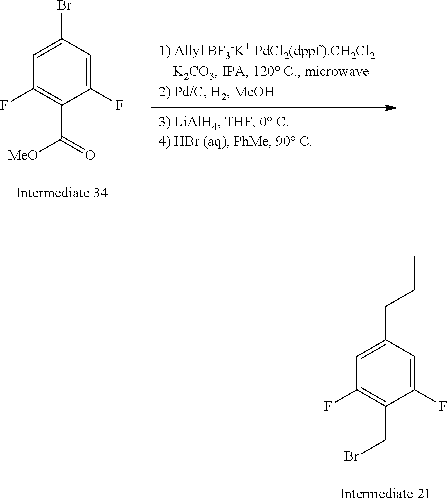

Step 1) A mixture of methyl 4-bromo-2,6-difluorobenzoate (Intermediate 34, 1.50 g total, 5.98 mmol), potassium allyltrifluoroborate (2.65 g total, 17.9 mmol) and K₂CO₃ (2.47 g total, 17.9 mmol) in IPA (20 mL total) was degassed with N₂ at rt for 15 min. [1,1′-bis(diphenylphosphino)ferrocene]dichloropalladium(II), complex with dichloromethane (0.25 g total, 0.31 mmol) was added and the reaction mixture was heated in three equal scale batches at 120° C. in a microwave reactor for 50 min. The combined reaction mixture was partitioned between water (100 mL) and EtOAc (80 mL) and the aqueous phase was extracted with EtOAc (2×50 mL). The combined organic phases were dried (Na₂SO₄) and concentrated in vacuo to yield methyl 2,6-difluoro-4-[prop-1-en-1-yl]benzoate (1.10 g, 5.18 mmol) as a colorless oil.

LCMS (Method 7): m/z 213.5 (ES+), at 4.67 min.

Step 2) 10% Palladium on carbon (10% wet, 0.25 g) was added to a solution of methyl 2,6-difluoro-4-[prop-1-en-1-yl]benzoate (1.00 g, 4.71 mmol) in anhydrous MeOH (15 mL) under a N₂ atmosphere at rt. The reaction mixture was then stirred at rt under an atmosphere of H₂ for 2 h. After filtration through celite the filtrate was concentrated in vacuo and purification by gradient flash column chromatography eluting with 0-1% EtOAc in hexane yielded methyl 2,6-difluoro-4-propylbenzoate (0.90 g, 4.20 mmol) as a colorless liquid.

LCMS (Method 7): m/z 215.5 (ES+), at 4.76 min.

¹H NMR: (400 MHz, DMSO-d₆) δ: 0.88 (t, J=7.3, 3H), 1.59 (dt, J=14.8, 7.4, 2H), 2.61 (t, J=7.6, 2H), 3.87 (s, 3H), 7.14 (d, J=9.9, 2H).

Step 3) LiAlH₄ (1M in THF, 5.10 mL, 5.10 mmol) was added to a solution of methyl 2,6-difluoro-4-propylbenzoate (0.90 g, 4.20 mmol) in anhydrous THF (15 mL) at 0° C. and the reaction mixture was stirred at 0° C. for 1 h before the addition of saturated aqueous of NH₄Cl (100 mL) and EtOAc (80 mL). The phases were separated, the aqueous layer was extracted with EtOAc (3×50 mL), the combined organic phases were dried (Na₂SO₄) and concentrated in vacuo. Purification by gradient flash column chromatography eluting with 0-15% EtOAc in hexane yielded (2,6-difluoro-4-propylphenyl)methanol (0.50 g, 2.69 mmol) as a colorless liquid.

LCMS (Method 7): m/z 204.5 (ES+, M+18), at 4.56 min.

¹H NMR: (400 MHz, DMSO-d₆) δ: ppm 0.88 (t, J=7.3, 3H), 1.51-1.65 (m, 2H), 2.51-2.60 (m, 2H), 4.45 (dt, J=5.7, 1.4, 2H), 5.16 (t, J=5.6, 1H), 6.85-6.98 (m, 2H).

Step 4) 48% Aqueous HBr (2 mL) was added to a solution of (2,6-difluoro-4-propylphenyl) methanol (0.25 g, 1.34 mmol) in toluene (5 mL) at rt and the reaction mixture was heated at 90° C. for 4 h. The toluene layer containing the title compound was separated from reaction mixture and used as such in the synthesis of Examples 28 and 34.

### Data in Table 1

### Intermediate 24, 5-bromo-2-(bromomethyl)-1-fluoro-3-methylbenzene

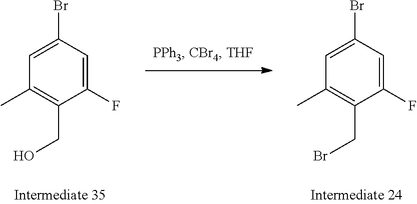

The title compound (486 mg, 1.72 mmol) was prepared from (4-bromo-2-fluoro-6-methylphenyl)methanol (Intermediate 35, 404 mg, 1.84 mmol), CBr₄ (735 mg, 2.22 mmol) and triphenylphosphine (675 mg, 2.57 mmol) in DCM (8 mL) at 0° C. for 10 min, then rt overnight, using the methods of Intermediate 18, Step 2.

### Data in Table 1

### Intermediate 28, 4-(bromomethyl)-3,5-difluorobenzyl (2H₃)methylether

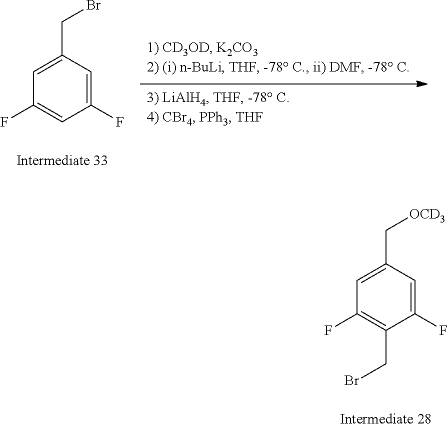

Step 1) 1,3-Difluoro-5-{[(²H₃)methyloxy]methyl}benzene (1.80 g, 11.2 mmol) was prepared from 1-(bromomethyl)-3,5-difluorobenzene (Intermediate 33, 3.50 g, 16.9 mmol) and K₂CO₃ (7.03 g, 50.9 mmol) in CD₃OD (35 mL) at rt, using the methods of Intermediate 19.

LCMS (Method 7): m/z not observed, at 4.33 min.

Step 2) 2,6-Difluoro-4-{[(2H₃)methyloxy]methyl}benzaldehyde (1.50 g, 7.93 mmol) was prepared from 1,3-difluoro-5-{[(²H₃)methyloxy]methyl}benzene (1.80 g, 11.2 mmol), n-BuLi (1.6 M in hexane, 20.0 mL, 32.0 mmol) and DMF (2.60 mL, 33.6 mmol) at −78° C. using the methods of Intermediate 19.

LCMS (Method 7): m/z 190.4 (ES+), at 3.64 min.

¹H NMR: (400 MHz, DMSO-d₆) δ: ppm 4.51 (s, 2H), 7.19 (d, J=10.0, 2H), 10.19 (s, 1H).

Step 3) (2,6-Difluoro-4-{[(²H₃)methyloxy]methyl}phenyl)methanol (1.10 g, 5.78 mmol) was prepared from 2,6-difluoro-4-{[(²H₃)methyloxy]methyl}benzaldehyde (1.50 g, 7.93 mmol) and LiAlH₄ (1M in THF, 10.0 mL, 10.0 mmol) in anhydrous THF (15 mL) at −78° C. using the methods of Intermediate 19.

LCMS (Method 7): m/z 209.4 (ES+, M+18), at 3.52 min.

¹H NMR: (400 MHz, DMSO-d₆) δ: ppm 4.42 (s, 2H), 4.48 (d, J=5.6, 2H), 5.24 (t, J=5.6, 1H), 6.98-7.06 (m, 2H).

Step 4) The title compound (422 mg, 1.68 mmol) was prepared as a yellow oil from (2,6-difluoro-4-{[(²H₃)methyloxy]methyl}phenyl)methanol (382 mg, 2.00 mmol), CBr₄ (995 mg, 3.00 mmol) and triphenylphosphine (787 mg, 3.00 mmol) in THF (6 mL) at 0° C. for 30 min, then rt overnight, using the methods of Intermediate 18.

### Data in Table 1

### Intermediate 29, 4-(bromomethyl)-3,5-difluorophenyl (²H₃)methyl ether

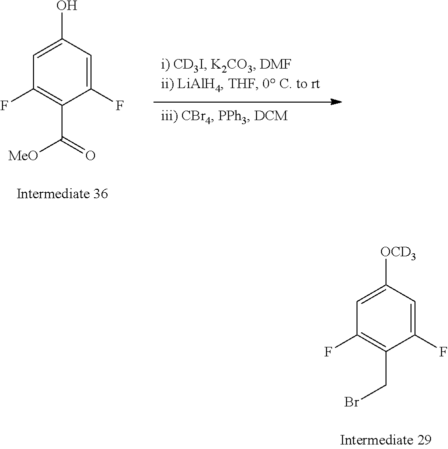

Step 1) Iodomethane-d₃ (0.40 mL, 6.43 mmol) and K₂CO₃ (880 mg, 6.37 mmol) were added to a solution of methyl 2,6-difluoro-4-hydroxybenzoate (Intermediate 36, 600 mg, 3.19 mmol) in DMF (1 mL) and the resulting thick suspension was stirred at rt overnight. Aqueous NH₃ (28%, 10 mL), water (50 mL) and EtOAc (50 mL) were added and the phases were separated. The organic phase was washed with brine (50 mL), the aqueous phase was extracted with EtOAc (50 mL) and this organic phase was washed with brine (50 mL). The combined organic phases were concentrated in vacuo to yield methyl 2,6-difluoro-4-[(2H₃)methyloxy]benzoate (703 mg, 3.43 mmol) as an off-white powder.

LCMS (Method 1): m/z 206.0 (ES+), at 1.92 min.

¹H NMR: (400 MHz, CDCl₃) δ: ppm 3.91 (s, 3H), 6.42-6.52 (m, 2H).

Step 2) A solution of methyl 2,6-difluoro-4-[(2H₃)methyloxy]benzoate (700 mg, 3.41 mmol) in THF (7 mL) was added to LiAlH₄ (1M in THF, 6.80 mL, 6.80 mmol) at 0° C., and the mixture was stirred at rt for 24 h. After cooling to 0° C., EtOAc (10 mL) was cautiously added, followed by water (5 mL) and aqueous HCl (1M, 5 mL). After removal of the THF in vacuo further EtOAc and water were added and the phases were separated. The organic phase was washed with saturated aqueous NaHCO₃ and concentrated in vacuo to yield {2,6-difluoro-4-[(²H₃)methyloxy]phenyl}methanol (505 mg) as a clear oil which was used in the next step without further purification.

¹H NMR: (400 MHz, CDCl₃) δ: ppm 1.73 (t, J=6.3, 1H), 4.69 (d, J=6.2, 2H), 6.40-6.51 (m, 2H).

Step 3) The title compound (361 mg, 1.50 mmol) was prepared from crude {2,6-difluoro-4-[(2H₃)methyloxy]phenyl}methanol (505 mg), CBr₄ (1.14 g, 3.44 mmol) and triphenylphosphine (1.05 g, 4.00 mmol) in DCM (8 mL) at rt for 24 h, using the methods of Intermediate 18.

### Data in Table 1

### Intermediate 30, 4-(bromomethyl)-2,3,5-trifluorophenyl methyl ether

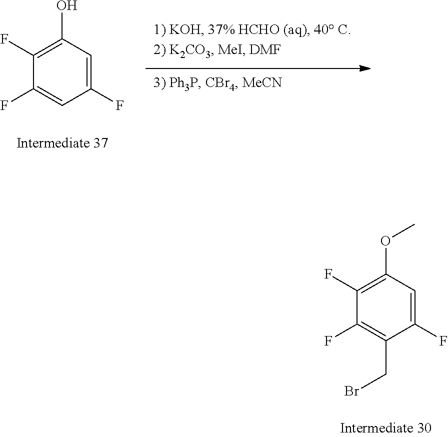

Step 1) KOH (0.79 g, 14.1 mmol) was added to a solution of 2,3,5-trifluorophenol (Intermediate 37, 1.90 g, 12.8 mmol) in water (20 mL) and the mixture was heated to 60° C. before the dropwise addition of 37% aqueous formaldehyde (1.43 mL, 19.3 mmol). The mixture was heated at 40° C. for 16 h before cooling to rt and the addition of concentrated aqueous HCl (20 mL) and stirring for 10 min. The resulting precipitate was filtered and washed with water to yield 2,3,5-trifluoro-4-(hydroxymethyl)phenol (1.30 g, 7.30 mmol) as a white solid.

LCMS (Method 3): m/z 177 (ES−), at 0.68 min.

¹H NMR: (400 MHz, DMSO-d₆) δ: 4.41 (dt, J=1.6, 5.7, 2H), 5.17 (t, J=5.6, 1H), 6.62 (ddd, J=2.3, 6.7, 10.9, 1H), 10.84 (s, 1H).

Step 2) K₂CO₃ (1.01 g, 7.31 mmol) and methyl iodide (0.46 mL, 7.39 mmol) were added to a solution of 2,3,5-trifluoro-4-(hydroxymethyl)phenol (1.30 g, 7.30 mmol) in DMF (10 mL) and the mixture was stirred at rt for 16 h. Water (100 mL) and EtOAc (100 mL) were added and the phases were separated. The organic phase was dried (MgSO₄), filtered and concentrated in vacuo to yield crude (2,3,6-trifluoro-4-methoxyphenyl)methanol (1.11 g, 5.78 mmol) as a white solid.

LCMS (Method 3): m/z not observed, at 0.93 min.

¹H NMR: (400 MHz, DMSO-d₆) δ: ppm 3.87 (s, 3H), 4.45 (dt, J=1.7, 5.8, 2H), 5.24 (t, J=5.6, 1H), 7.04 (ddd, J=2.3, 6.7, 11.4, 1H).

Step 3: Triphenylphosphine (1.82 g, 6.94 mmol) and CBr₄ (2.30 g, 6.94 mmol) were added to a solution of (2,3,6-trifluoro-4-methoxyphenyl)methanol (1.11 g, 5.78 mmol) in anhydrous MeCN (20 mL) and the reaction mixture was stirred at rt for 16 h. Water (50 mL) and EtOAc (50 mL) were added and the phases were separated. The organic phase was dried (MgSO₄), filtered and concentrated in vacuo before purification by gradient flash column chromatography, eluting with 0-5% TBME in isohexane yielded the title compound (0.56 g, 2.20 mmol) as a colourless oil.

### Data in Table 1

### Preparation of Additional Intermediates

### Intermediate 39, 2-bromo-5-fluoro-3,4-dihydronaphthalen-1-yl acetate

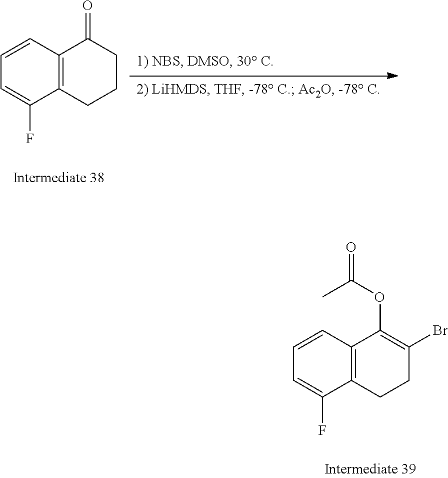 [SMILES: O=C1CCCc2c(F)cccc21 ; CC(=O)OC1=C(Br)CCc2c(F)cccc21]

Step 1) NBS (9.68 g, 54.4 mmol) was added in portions to 5-fluoro-3,4-dihydronaphthalen-1(2H)-one (Intermediate 38, 6.07 g, 37.0 mmol) in DMSO (125 mL) under a nitrogen atmosphere and stirred at 30° C. for 18 h. The reaction mixture was partitioned between water (600 mL) and hexane (300 mL). The organic phase was separated and the aqueous phase extracted with hexane (300 mL). The combined organic phases were washed with water (600 mL), dried (MgSO₄), and concentrated in vacuo. Purification by gradient flash chromatography, eluting with 0-5% EtOAc in petroleum ether yielded 2-bromo-5-fluoro-3,4-dihydronaphthalen-1(2H)-one (5.24 g, 21.6 mmol) as a light orange solid.

LCMS (Method 2): m/z not observed, at 1.91 min.

¹H NMR: (400 MHz, CDCl₃) δ: ppm 2.48-2.55 (m, 2H), 3.00-3.20 (m, 2H), 4.72 (t, J=4.0, 1H), 7.25-7.38 (m, 2H), 7.90 (dd, J=1.4, 7.6, 1H).

Step 2) LiHMDS (1M in THF) (43.4 mL, 43.4 mmol) was added dropwise to a solution of 2-bromo-5-fluoro-3,4-dihydronaphthalen-1(2H)-one (5.22 g, 21.7 mmol) in THF (105 mL) at −78° C. After 30 minutes at −78° C., acetic anhydride (6.14 mL, 65.0 mmol) was added dropwise at −78° C. and the reaction was allowed to stir at −78° C. for 2 h. The reaction mixture was partitioned between aqueous HCl (1N, 118 mL) and EtOAc (85 mL) and the aqueous layer was extracted with EtOAc (3×60 mL). The combined organic layers were dried (Na₂SO₄) and concentrated in vacuo. Purification by gradient flash chromatography, eluting with 5-10% EtOAc in petroleum ether, yielded the title compound (5.08 g, 17.8 mmol) as an off white solid.

### Data in Table 1

### Intermediate 40, (2-fluoro-6-nitrophenyl)acetaldehyde, Route 1

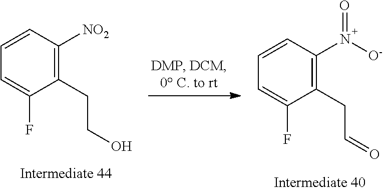

DMP (2.48 g, 5.85 mmol) was added portion wise to a solution of 2-(2-fluoro-6-nitrophenyl)ethan-1-ol (Intermediate 44, 0.50 g, 2.70 mmol) in DCM (7 mL) at 0° C. After stirring at rt for 48 h the reaction mixture was filtered through a pad of celite and washed with 5% DCM in hexane. The combined filtrate was washed with saturated aqueous NaHCO₃ (10 mL) and brine (10 mL), dried (Na₂SO₄) and concentrated in vacuo to yield the title compound (0.41 g) as a brown liquid which was used in the subsequent step without further purification.

### Data in Table 1

### Intermediate 40, (2-fluoro-6-nitrophenyl)acetaldehyde, Route 2

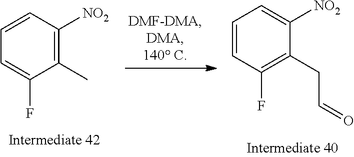

1-Fluoro-2-methyl-3-nitrobenzene (Intermediate 42, 20.0 g, 129 mmol) and 1,1-dimethoxy-N,N-dimethylmethanamine (30.9 g, 259 mmol) was dissolved in DMF (200 mL) and stirred at 140° C. for 2 h. The reaction mixture was cooled to rt, poured into aqueous HCl (4N, 400 mL), and stirred at rt for 3 h, before extraction with EtOAc (2×150 mL). The combined organic phases were washed with brine (75 mL), dried (Na₂SO₄), and concentrated in vacuo. Purification by flash column chromatography eluting with 5% EtOAc in petroleum ether yielded the title compound (14.2 g, 77.5 mmol) as dark red liquid.

### Data in Table 1

TABLE 1

| Intermediate | Name | Structure | Data |
|---|---|---|---|
| 1 | 3-[3-(1,1- difluoroethyl)-4- methylphenyl]-6- fluoro-1,3,4,5- tetrahydro-2H-1,3- benzodiazepin-2-one | 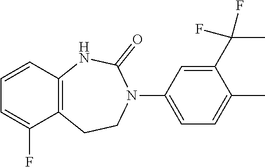 [SMILES: Cc1ccc(N2CCc3c(F)cccc3NC2=O)cc1C(C)(F)F] | LCMS (Method 7): m/z 335.3 (ES+), at 4.27 min ¹H NMR: (400 MHz, DMSO-d₆) δ: 1.99 (t, J = 19.1, 3H), 2.42 (s, 3H), 2.96-3.04 (m, 2H), 3.86-3.94 (m, 2H), 6.76 (dd, J = 8.1, 9.5, 1H), 6.98 (d, J = 8.3, 1H), 7.10-7.18 (m, 1H), 7.32 (d, J = 2.5, 2H), 7.40 (d, J = 1.9, 1H), 9.07 (s, 1H) |
| 2 | 6-fluoro-3-[4-methyl- 3- (methylsulfanyl)phenyl]- 1,3,4,5-tetrahydro- 2H-1,3- benzodiazepin-2-one | 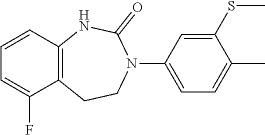 [SMILES: CSc1cc(N2CCc3c(F)cccc3NC2=O)ccc1C] | LCMS (Method 5): m/z 317.0 (M + H)+ (ES+), at 2.08 min, 88% ¹H NMR: (400 MHz, DMSO-d₆) δ: 2.23 (s, 3H), 2.45 (s, 3H), 2.96-3.03 (m, 2H), 3.84-3.91 (m, 2H), 6.74 (t, J = 8.8, 1H), 6.93-7.01 (m, 2H), 7.02-7.23 (m, 3H), 8.98 (s, 1H) |
| 3 | 6-fluoro-3-[4-methyl- 3- (methylsulfonyl)phenyl]- 1,3,4,5-tetrahydro- 2H-1,3- benzodiazepin-2-one | 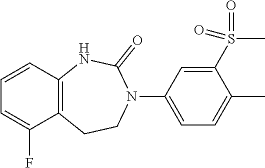 [SMILES: Cc1ccc(N2CCc3c(F)cccc3NC2=O)cc1S(C)(=O)=O] | LCMS (Method 7): m/z 349.1 (ES+), at 3.69 min ¹H NMR: (500 MHz, DMSO-d₆) δ: 2.63 (s, 3H), 2.98-3.04 (m, 2H), 3.23 (s, 3H), 3.91-3.97 (m, 2H), 6.76 (ddd, J = 1.2, 8.1, 9.3, 1H), 6.99 (d, J = 8.3, 1H), 7.10-7.19 (m, 1H), 7.46 (d, J = 8.3, 1H), 7.58 (dd, J = 2.4, 8.1, 1H), 7.83 (d, J = 2.4, 1H), 9.12 (s, 1H) |
| 4 | 3-[2,4-dimethyl-5- (methylsulfonyl)phenyl]- l ,3,4,5-tetrahydro- 2H-1,3- benzodiazepin-2-one | 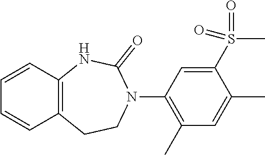 [SMILES: Cc1cc(C)c(S(C)(=O)=O)cc1N1CCc2ccccc2NC1=O] | MS (ESI + ve): 345 ¹H-NMR (400 MHz; CDCl₃) δ: 2.31 (s, 3H), 2.66 (s, 3H), 3.07 (s, 3H), 3.20 (t, J = 6.2, 2H), 3.84 (t, J = 6.2, 2H), 6.69 (s, 1H), 6.80 (d, J = 8.0, 1H), 6.98 (t, J = 7.4, 1H), 7.10-7.18 (m, 2H), 7.19 (d, J = 7.8, 1H), 7.84 (bs, 1H) |
| 5 | 3-[2,4-dimethyl-5- (methylsulfonyl)phenyl]- 6-fluoro-1,3,4,5- tetrahydro-2H-1,3- benzodiazepin-2-one | 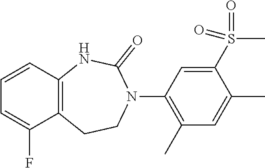 [SMILES: Cc1cc(C)c(S(C)(=O)=O)cc1N1CCc2c(F)cccc2NC1=O] | LCMS (Method 7): m/z 363.2 (ES+), at 3.78 min ¹H NMR: (400 MHz, DMSO-d₆) δ: 2.20 (s, 3H), 2.61 (s, 3H), 2.98 (br s, 1H), 3.05 (br s, 1H), 3.23 (s, 3H), 3.84 (t, J = 5.2, 2H), 6.77 (ddd, J = 1.1, 8.1, 9.4, 1H), 6.99 (d, J = 8.3, 1H), 7.12-7.19 (m, 1H), 7.39 (s, 1H), 7.69 (s, 1H), 9.16 (d, J = 1.4, 1H) |
| 6 | 3-[5-(1,1- difluoroethyl)-6- methylpyridin-3-yl]-6- fluoro-1,3,4,5- tetrahydro-2H-1,3- benzodiazepin-2-one | 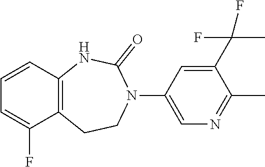 [SMILES: Cc1ncc(N2CCc3c(F)cccc3NC2=O)cc1C(C)(F)F] | LCMS (Method 7): m/z 336.3 (ES+), at 4.88 min ¹H NMR: (400 MHz, DMSO-d₆) δ: 2.04 (t, J = 19.2, 3H), 2.61 (s, 3H), 2.99-3.08 (m, 2H), 3.91- 4.00 (m, 2H), 6.78 (ddd, J = 1.2, 8.2, 9.4, 1H), 7.00 (dd, J = 1.2, 8.4, 1H), 7.12-7.19 (m, 1H), 7.85 (d, J = 2.4, 1H), 8.54 (d, J = 2.4, 1H), 9.21 (d, J = 1.3, 1H) |
| 7 | 3-[5-(1,1- difluoroethyl)-6- methylpyridin-3-yl]-6- fluoro-1,3,4,5- tetrahydro-2H-1- benzazepin-2-one | 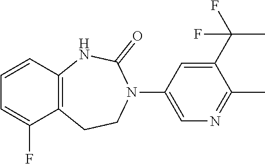 [SMILES: Cc1ncc(N2CCc3c(F)cccc3NC2=O)cc1C(C)(F)F] | LCMS (Method 2): m/z 335.0 (ES+), at 1.74 min ¹H NMR: (500 MHz, DMSO-d₆) δ: 1.99 (t, 7 19.1, 3H), 2.25 (tt, J = 7.2, 13.0, 1H), 2.44-2.51 (m, 1H), 2.57 (t, J = 2.1, 3H), 2.64 (td, J = 7.4, 13.7, 1H), 3.07 (dd, J = 6.5, 13.9, 1H), 3.75 (dd, J = 7.7, 12.4, 1H), 6.90 (dd, J = 1.1, 8.0, 1H), 7.04 (ddd, J = 1.1, 8.4, 9.3, 1H), 7.28-7.34 (m, 1H), 7.79 (d, J = 2.1, 1H), 8.43 (d, J = 2.1, 1H), 10.02 (s, 1H) |
| 8 | 3-[5- (difluoromethoxy)-6- methylpyridin-3-yl]-6- fluoro-1,3,4,5- tetrahydro-2H-1,3- benzodiazepin-2-one | 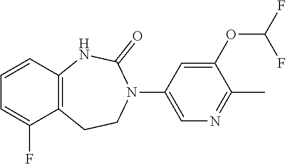 [SMILES: Cc1ncc(N2CCc3c(F)cccc3NC2=O)cc1OC(F)F] | LCMS (Method 7): m/z 338.3 (ES+), at 2.18 min ¹H NMR: (400 MHz, DMSO-d₆) δ: 2.43 (s, 3H), 3.00-3.08 (m, 2H), 3.90-4.00 (m, 2H), 6.78 (ddd, J = 1.2, 8.2, 9.3, 1H), 7.00 (dt, J = 1.1, 8.3, 1H), 7.11-7.21 (m, 1H), 7.29 (t, J = 73.6, 1H), 7.69 (d, J = 2.1, 1H), 8.36 (d, J = 2.1, 1H), 9.21 (d, J = 1.4, 1H) |
| 9 | 6-fluoro-3-[6-methyl- 5- (methylsulfonyl)pyridin- 3-yl]-1,3,4,5- tetrahydro-2H-1- benzazepin-2-one | 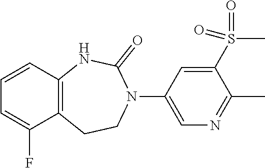 [SMILES: Cc1ncc(N2CCc3c(F)cccc3NC2=O)cc1S(C)(=O)=O] | LCMS (Method 2): m/z 349.1 (ES+), at 1.47 min ¹H NMR: (500 MHz, CDCl₃) δ: 2.28-2.46 (m, 1H), 2.49-2.60 (m, 1H), 2.74-2.85 (m, 1H), 2.91 (s, 3H), 3.13 (s, 3H), 3.29 (dd, J = 6.7, 14.2, 1H), 3.77 (dd, J = 7.6, 12.4, 1H), 6.87 (d, J = 7.9, 1H), 7.01 (t, J = 8.6, 1H), 7.23-7.32 (m, 1H), 7.44 (s, 1H), 8.23 (d, J = 2.3, 1H), 8.62 (d, J = 2.2, 1H) |
| 10 | 3-{5-(1,1- difluoroethyl)-6- [(²H₃)methyloxy]pyridin- 3-yl}-6-fluoro- 1,3,4,5-tetrahydro-2H- 1,3-benzodiazepin-2- one | 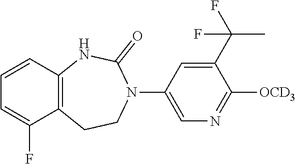 | LCMS (Method 9): m/z 355.2 (ES+), at 3.10 min ¹H NMR: (400 MHz, DMSO-d₆) δ: ppm 2.01 (t, J = 19.2, 3H), 3.02-3.06 (m, 2H), 3.84-3.90 (m, 2H), 6.76 (t, J = 8.6, 1H), 6.98 (d, J = 8.4, 1H), 7.10-7.18 (m, 1H), 7.85 (d, J = 2.4, 1H), 8.25 (d, J = 2.4, 1H), 9.1 (s, 1H) |
| 11 | 3-[5-(1,1- difluoroethyl)-6- methoxypyridin-3-yl]- 6-fluoro-1,3,4,5- tetrahydro-2H-1,3- benzodiazepin-2-one | 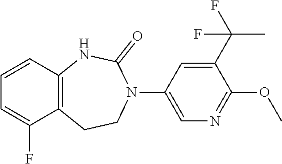 [SMILES: COc1ncc(N2CCc3c(F)cccc3NC2=O)cc1C(C)(F)F] | LCMS (Method 7): m/z 352.3 (ES+), at 2.26 min ¹H NMR: (400 MHz, DMSO-d₆) δ: 2.01 (t, J = 19.2, 3H), 3.00-3.08 (m, 2H), 3.84-3.91 (m, 2H), 3.96 (s, 3H), 6.77 (t, J = 8.8, 1H), 6.99 (d, J = 8.2, 1H), 7.15 (td, J = 6.5, 8.1, 1H), 7.86 (d, J = 2.6, 1H), 8.26 (d, J = 2.6, 1H), 9.16 (s, 1H) |
| 12 | 3-[5-(1,1- difluoroethyl)-6- methoxypyridin-3-yl]- 6-fluoro-1,3,4,5- tetrahydro-2H-1- benzazepin-2-one | 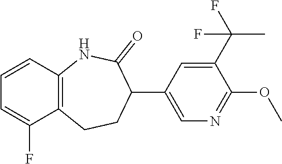 [SMILES: COc1ncc(C2CCc3c(F)cccc3NC2=O)cc1C(C)(F)F] | LCMS (Method 3): m/z 351 (ES+), at 1.50 min ¹H NMR: (400 MHz, DMSO-d₆) δ: 1.99 (t, J = 19.1, 3H), 2.24 (tt, J = 7.0, 13.2, 1H), 2.44 (dt, J = 6.1, 12.7, 1H), 2.56-2.68 (m, 1H), 3.07 (dd, J = 6.6, 13.8, 1H), 3.72 (dd, J = 7.6, 12.3, 1H), 3.91 (s, 3H), 6.90 (d, J = 7.9, 1H), 7.05 (ddd, J = 1.1, 8.4, 9.4, 1H), 7.27-7.34 (m, 1H), 7.85 (d, J = 2.3, 1H), 8.12 (d, J = 2.3, 1H), 10.02 (s, 1H) |
| 13 | 6-fluoro-3-[6- methoxy-5- (methylsulfonyl)pyridin- 3-yl]-1,3,4,5- tetrahydro-2H-1,3- benzodiazepin-2-one | 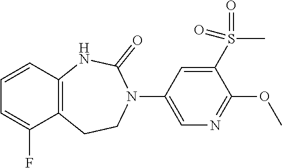 [SMILES: COc1ncc(N2CCc3c(F)cccc3NC2=O)cc1S(C)(=O)=O] | LCMS (Method 7): m/z 366.2 (ES+), at 1.88 min ¹H NMR: (400 MHz, DMSO-d₆) δ: 3.02-3.10 (m, 2H), 3.33 (s, 3H), 3.87-3.94 (m, 2H), 4.06 (s, 3H), 6.78 (t, J = 8.8, 1H), 6.99 (d, J = 8.3, 1H), 7.15 (q, J = 7.8, 1H), 8.17 (d, J = 2.7, 1H), 8.48 (d, J = 2.6, 1H), 9.24 (s, 1H) |
| 14 | 6-fluoro-3-[6- methoxy-5- (methylsulfonyl)pyridin- 3-yl]-1,3,4,5- tetrahydro-2H-1- benzazepin-2-one | 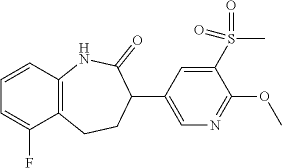 [SMILES: COc1ncc(C2CCc3c(F)cccc3NC2=O)cc1S(C)(=O)=O] | LCMS (Method 2): m/z 365.1 (ES+), at 1.64 min ¹H NMR: (500 MHz, DMSO-d₆) δ: 2.27 (tt, J = 7.2, 13.1, 1H), 2.36-2.44 (m, 1H), 3.03-3.11 (m, 1H), 3.28 (s, 3H), 3.83 (dd, J = 7.7, 12.3, 1H), 4.01 (s, 3H), 6.90 (dd, J = 1.0, 8.0, 1H), 7.05 (t, J = 8.6, 1H), 7.28-7.34 (m, 1H), 8.19 (d, J = 2.4, 1H), 8.33 (d, J = 2.3, 1H), 10.05 (s, 1H). One proton obscured by the solvent peak |
| 15 | 3-[5-(1,1- difluoroethyl)-2,6- dimethoxypyridin-3- yl]-6-fluoro-1,3,4,5- tetrahydro-2H-1,3- benzodiazepin-2-one | 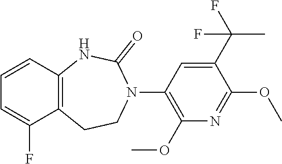 [SMILES: COc1nc(OC)c(C(C)(F)F)cc1N1CCc2c(F)cccc2NC1=O] | LCMS (Method 6): m/z 382.0 (ES+), at 1.80 min ¹H NMR: (400 MHz, CDCl₃) δ: 1.95 (d, J = 18.7, 3H), 3.09-3.16 (m, 2H), 3.74-3.82 (m, 2H), 3.98 (s, 3H), 4.00 (s, 3H), 6.50-6.59 (m, 2H), 6.67-6.76 (m, 1H), 7.05-7.15 (m, 1H), 7.64 (s, 1H) |
| 16 | 3-[2,6-dimethoxy-5- (methylsulfonyl)pyridin- 3-yl]-6-fluoro- 1,3,4,5-tetrahydro-2H- 1,3-benzodiazepin-2- one | 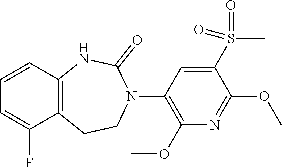 [SMILES: COc1nc(OC)c(S(C)(=O)=O)cc1N1CCc2c(F)cccc2NC1=O] | LCMS (Method 10): m/z 396.0 (ES+), at 1.91 min ¹H NMR: (400 MHz, DMSO-d₆) δ: 2.94-3.06 (m, 2H), 3.22 (s, 3H), 3.64-3.73 (m, 2H), 3.98 (s, 3H), 4.06 (s, 3H), 6.73 (t, J = 8.9, 1H), 6.95 (d, J = 8.2, 1H), 7.08-7.15 (m, 1H), 7.94 (s, 1H), 9.10 (s, 1H) |
| 17 | 5-bromo-2- (bromomethyl)-1,3- difluorobenzene | 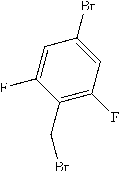 [SMILES: Fc1cc(Br)cc(F)c1CBr] | Commercially available, CAS 162744-60-7 |
| 18 | 4-(bromomethyl)-3,5- difluorophenyl propan-2-yl ether | 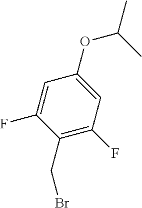 | ¹H NMR: (400 MHz, DMSO-d₆) δ: ppm 1.25 (d, J = 5.9, 6H), 4.60 (s, 2H), 4.64-4.72 (m, 1H), 6.77 (d, J = 10.0, 2H) |
| 19 | [2,6-difluoro-4- (methoxymethyl) phenyl]methanol | 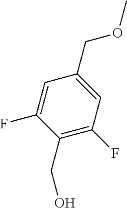 | LCMS (Method 7): m/z 206.4 (ES+, M + 18), at 4.94 min ¹H NMR: (400 MHz, DMSO-d₆) δ: 3.31 (s, 3H), 4.40-4.54 (m, 4H), 5.24 (t, J = 5.7, 1H), 6.97-7.07 (m, 2H) |
| 20 | 4-(bromomethyl)-3,5- difluorobenzyl methyl ether | 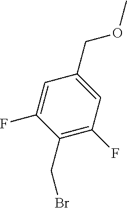 | LCMS (Method 1): m/z not observed, at 4.14 min ¹H NMR: (400 MHz, CDCl₃) δ: 3.41 (s, 3H), 4.42 (s, 2H), 4.52 (s, 2H), 6.85-6.94 (m, 2H) |
| 21 | 2-(bromomethyl)-1,3- difluoro-5- propylbenzene | 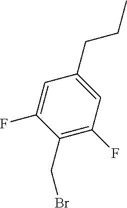 | TLC: Rf 0.7, EtOAc/Hexane 1:9 |
| 22 | 4-(bromomethyl)-3,5- difluorophenyl methyl ether | 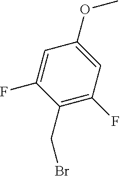 | Commercially available, CAS 94278-68-9 |
| 23 | 2-(bromomethyl)-5- chloro-1,3- difluorobenzene | 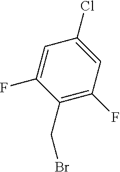 [SMILES: Fc1cc(Cl)cc(F)c1CBr] | Commercially available, CAS 537013-52-8 |
| 24 | 5-bromo-2- (bromomethyl)-1- fluoro-3- methylbenzene | 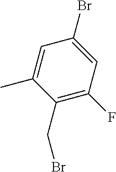 | ¹H NMR: (400 MHz, CDCl₃) δ: ppm 2.40 (s, 3H), 4.49 (d, J = 1.6, 2H), 7.07-7.19 (m, 1H), 7.26 (d, J = 0.7, 1H) |
| 25 | 2-(bromomethyl)-1,5- difluoro-3- methylbenzene | 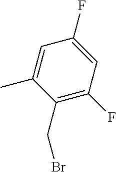 | Commercially available, CAS 1803735-07-0 |
| 26 | 4-bromo-1- (bromomethyl)-2- fluorobenzene | 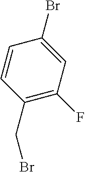 | Commercially available, CAS 76283-09-5 |
| 27 | 4-(bromomethyl)-3- fluorophenyl methyl ether | 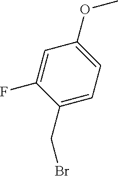 | Commercially available, CAS 54788-19-1 |
| 28 | 4-(bromomethyl)-3,5- difluorobenzyl (²H₃)methyl ether | 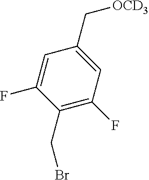 | LCMS (Method 1): m/z not observed, at 4.23 min ¹H NMR: (400 MHz, CDCl₃) δ: 4.42 (s, 2H), 4.52 (s, 2H), 6.82-6.96 (m, 2H) |
| 29 | 4-(bromomethyl)-3,5- difluorophenyl (²H₃)methyl ether | 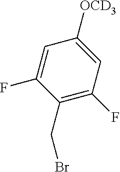 | ¹H NMR: (400 MHz, CDCl₃) δ: ppm 4.51 (s, 2H), 6.43-6.49 (m, 2H) |
| 30 | 4-(bromomethyl)- 2,3,5-trifluorophenyl methyl ether | 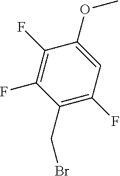 | LCMS (Method 11): m/z not observed, at 4.27 min ¹H NMR: (400 MHz, DMSO-d₆) δ: ppm 3.90 (s, 3H), 4.65 (d, J = 1.2, 2H), 7.15 (ddd, J = 2.3, 6.7, 11.6, 1H) |
| 31 | 3-(bromomethyl)-4- fluorobenzonitrile | 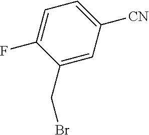 [SMILES: N#Cc1ccc(F)c(CBr)c1] | Commercially available, CAS 856935-35-8 |
| 32 | 3,5-difluoro-4- (hydroxymethyl)phenol | 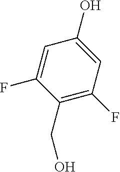 [SMILES: OCc1c(F)cc(O)cc1F] | Commercially available, CAS 438049-36-6 |
| 33 | 1-(bromomethyl)-3,5- difluorobenzene | 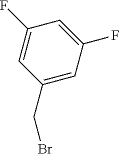 | Commercially available, CAS 141776-91-2 |
| 34 | methyl 4-bromo-2,6- difluorobenzoate | 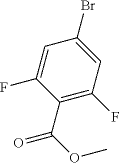 | Commercially available, CAS 773134-11-5 |
| 35 | (4-bromo-2-fluoro-6- methylphenyl)methanol | 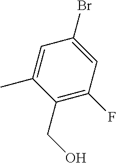 | Commercially available, CAS 1417736-81-2 |
| 36 | methyl 2,6-difluoro-4- hydroxybenzoate | 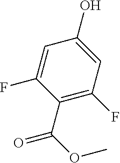 | Commercially available, CAS 194938-88-0 |
| 37 | 2,3,5-trifluorophenol | 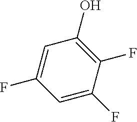 | Commercially available, CAS 2268-15-7 |
| 38 | 5-fluoro-3,4- dihydronaphthalen- 1(2H)-one | 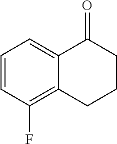 [SMILES: O=C1CCCc2c(F)cccc21] | Commercially available, CAS 93742-85-9 |
| 39 | 2-bromo-5-fluoro-3,4- dihydronaphthalen-1- yl acetate | 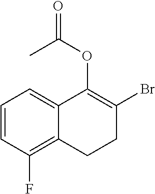 [SMILES: CC(=O)OC1=C(Br)CCc2c(F)cccc21] | LCMS (Method 2): m/z not observed, at 2.07 min ¹H NMR: (500 MHz, CDCl₃) δ: 2.35 (s, 3H), 2.93 (ddd, J = 1.3, 7.5, 8.9, 2H), 3.03 (t, J = 8.2, 2H), 6.88 (d, J = 7.6, 1H), 6.92-7.00 (m, 1H), 7.11-7.18 (m, 1H) |
| 40 | (2-fluoro-6- nitrophenyl)acetaldehyde | 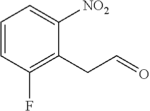 [SMILES: O=CCc1c(F)cccc1[N+](=O)[O-]] | LCMS (Method 9): m/z not observed, at 2.14 min ¹H NMR: (500 MHz, CDCl₃) δ: ppm 4.19 (s, 2H), 7.38-7.52 (m, 2H), 7.94 (br d, J = 8.4, 1H), 9.83 (s, 1H) |
| 41 | (2-fluoro-6- nitrophenyl)acetic acid | 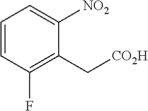 [SMILES: O=C(O)Cc1c(F)cccc1[N+](=O)[O-]] | Commercially available, CAS 136916-19-3 |
| 42 | 1-fluoro-2-methyl-3- nitrobenzene | 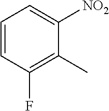 | Commercially available, CAS 769-10-8 |
| 43 | (2-nitrophenyl) acetaldehyde | 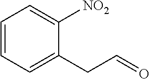 [SMILES: O=CCc1ccccc1[N+](=O)[O-]] | Commercially available, CAS 1969-73-9 |
| 44 | 2-(2-fluoro-6- nitrophenyl)ethan-1-ol | 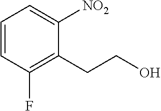 [SMILES: O=[N+]([O-])c1cccc(F)c1CCO] | Commercially available, CAS 118665-03-5 |
| 45 | 5-bromo-6-methoxy- 3-pyridinamine | 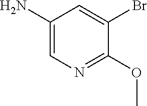 | Commercially available, CAS 53242-18-5 |
| 46 | 2-[6-methoxy-5- (methylsulfonyl)pyridin- 3-yl]-4,4,5,5- tetramethyl-1,3,2- dioxaborolane | 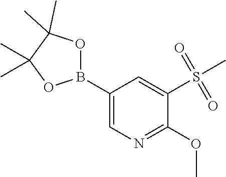 | LCMS (Method 2): m/z 314.0 (ES+), at 1.01 and 1.11 min ¹H NMR: (500 MHz, CDCl₃) δ: 1.33 (s, 12H), 3.20 (s, 3H), 4.15 (s, 3H), 8.61 (d, J = 1.9, 1H), 8.71 (d, J = 1.9, 1H) |
| 47 | 5-bromo-6- methylpyridin-3- amine | 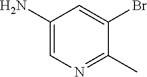 | Commercially available, CAS 186593-43-1 |
| 48 | 2-methyl-3- (methylsulfonyl)-5- (4,4,5,5-tetramethyl- 1,3,2-dioxaborolan-2- yl)pyridine | 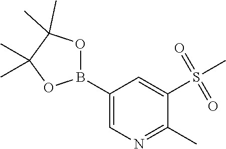 | ¹H NMR: (500 MHz, CDCl₃) δ: 1.35 (s, 12H), 2.96 (s, 3H), 3.12 (s, 3H), 8.66 (d, J = 1.8, 1H), 9.00 (d, J = 1.7, 1H) |
| 49 | 1-(5-bromo-2-methyl- 3-pyridinyl)ethanone | 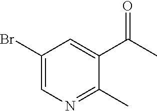 | Commercially available, CAS 1256823-89-8 |
| 50 | 3-(1,1-difluoroethyl)- 2-methyl-5-(4,4,5,5- tetramethyl-1,3,2- dioxaborolan-2- yl)pyridine | 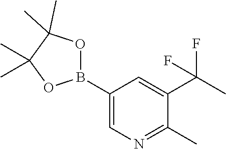 [SMILES: Cc1ncc(B2OC(C)(C)C(C)(C)O2)cc1C(C)(F)F] | LCMS (Method 2): m/z 284.1 (ES+), at 1.05 min ¹H NMR: (500 MHz, CDCl₃) δ: 1.35 (s, 12H), 1.97 (t, J = 18.3, 3H), 2.71 (t, J = 2.1, 3H), 8.09-8.13 (m, 1H), 8.85 (d, J = 1.4, 1H) |
| 51 | 3-bromo-2-methoxy- 5-nitropyridine | 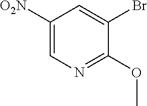 | Commercially available, CAS 15862-50-7 |
| 52 | 3-(1,1-difluoroethyl)- 2-methoxy-5-(4,4,5,5- tetramethyl-1,3,2- dioxaborolan-2- yl)pyridine | 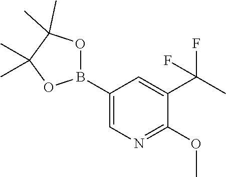 [SMILES: COc1ncc(B2OC(C)(C)C(C)(C)O2)cc1C(C)(F)F] | LCMS (Method 3): m/z 300 (ES+), at 1.78 min ¹H NMR: (400 MHz, DMSO-d₆) δ: 1.30 (s, 12H), 1.98 (t, J = 19.1, 3H), 3.97 (s, 3H), 7.89-7.99 (m, 1H), 8.47-8.52 (m, 1H) |
| 53 | 4-methyl-3- (methylsulfonyl)aniline | 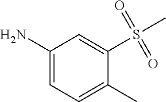 | Commercially available, CAS 307989-41-9 |
| 54 | 4-methyl-3- (methylsulfanyl) aniline | 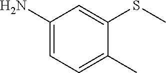 | Commercially available, CAS 98594-08-2 |
| 55 | 1-(2-methyl-5- nitrophenyl)ethanone | 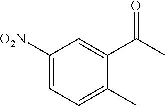 | Commercially available, CAS 58966-27-1 |
| 56 | 3-(1,1-difluoroethyl)- 4-methylaniline | 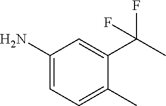 | LCMS (Method 7): m/z 172.2, at 3.13 min ¹H NMR: (400 MHz, CDCl₃) δ: 1.92 (t, J = 18.3, 3H), 2.36 (t, J = 2.4, 3H), 3.62 (s, 2H), 6.66 (dd, J = 2.5, 8.1, 1H), 6.84 (d, J = 2.5, 1H), 7.01 (d, J = 8.0, 1H) |
| 57 | 2,4-dimethyl-5- (methylsulfonyl)aniline | 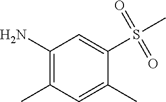 | Commercially available, CAS 849035-63-8 |
| 58 | 1-(2-methyl-5- nitropyridin-3-yl) ethanone | 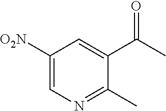 | Commercially available, CAS 68541-87-7 |
| 59 | 5-(1,1-difluoroethyl)- 6-methylpyridin-3- amine | 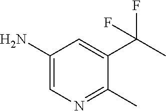 | LCMS (Method 7): m/z 173.4, at 2.81 min ¹H NMR: (400 MHz, DMSO-d₆) δ: 1.92 (t, J = 19.0, 3H), 2.39 (t, J = 2.3, 3H), 5.34 (s, 2H), 7.02 (d, J = 2.7, 1H), 7.88 (d, J = 2.6, 1H) |
| 60 | 5-bromo-3- (difluoromethoxy)-2- methylpyridine | 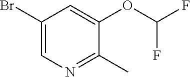 | Commercially available, CAS 1211536-99-0 |
| 61 | 5-(difluoromethoxy)- 6-methylpyridin-3- amine | 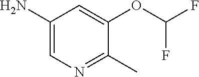 | LCMS (Method 7): m/z 175.5, at 3.55 min ¹H NMR: (400 MHz, DMSO-d₆) δ: 2.22 (s, 3H), 5.38 (s, 2H), 6.70-6.75 (m, 1H), 7.12 (t, J = 74.0, 1H), 7.70 (d, J = 2.3, 1H) |
| 62 | 3-bromo-2-chloro-5- nitropyridine | 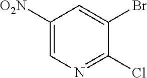 [SMILES: O=[N+]([O-])c1cnc(Cl)c(Br)c1] | Commercially available, CAS 5470-17-7 |
| 63 | 5-(1,1-difluoroethyl)- 6- [(²H₃)methyloxy]pyridin- 3-amine | 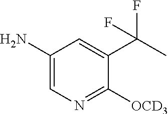 | LCMS (Method 12): m/z 192.1, at 3.84 min ¹H NMR: (400 MHz, CDCl₃) δ: 1.98 (t, J = 18.8, 3H), 3.45 (br s, 2H), 7.25 (d, J = 2.8, 1H), 7.70 (d, J = 2.8, 1H) |
| 64 | 3-bromo-2-methoxy- 5-nitropyridine | 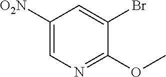 | Commercially available, CAS 15862-50-7 |
| 65 | 5-(1,1-difluoroethyl)- 6-methoxypyridin-3- amine | 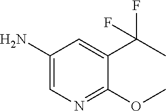 | LCMS (Method 7): m/z 189.1, at 1.40 min ¹H NMR: (400 MHz, CDCl₃) δ: 1.91 (d, J = 19.0, 3H), 3.79 (s, 3H), 5.03 (s, 2H), 7.16-7.22 (m, 1H), 7.58-7.62 (m, 1H) |
| 66 | 5-bromo-6-methoxy- 3-pyridinamine | 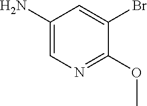 | Commercially available, CAS 53242-18-5 |
| 67 | 6-methoxy-5- (methylsulfonyl)pyridin- 3-amine | 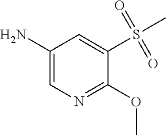 | LCMS (Method 7): m/z 203.4, at 8.29 min ¹H NMR: (400 MHz, CDCl₃) δ: 3.23 (s, 3H), 3.87 (s, 3H), 5.29 (s, 2H), 7.53 (d, J = 2.9, 1H), 7.79 (d, J = 3.0, 1H) |
| 68 | 2,6-dimethoxy-3- pyridinecarboxylic acid | 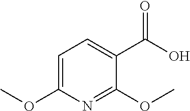 | Commercially available, CAS 16727-43-8 |
| 69 | 5-(1,1-difluoroethyl)- 2,6-dimethoxypyridin- 3-amine | 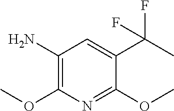 | LCMS (Method 6): m/z 219 (ES+), at 1.48 min ¹H NMR: (400 MHz, CDCl₃) δ: ppm 1.95 (t, J = 18.8, 3H), 3.41 (br s, 2H), 3.92 (s, 3H), 3.98 (s, 3H), 7.15 (s, 1H) |
| 70 | 6-methoxy-3- nitropyridin-2-amine | 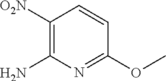 | Commercially available, CAS 73896-36-3 |
| 71 | 2,6-dimethoxy-5- (methylsulfonyl)pyridin- 3-amine | 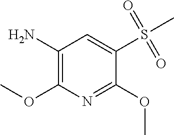 | LCMS (Method 8): m/z 233.1 (ES \| ), at 1.16 min ¹H NMR: (300 MHz, DMSO-d₆) δ: ppm 3.12 (s, 3H), 3.91 (s, 3H), 3.94 (s, 3H), 4.78 (br s, 2H), 7.36 (s, 1H) |

### SYNTHESIS OF EXAMPLES

Typical procedures for the preparation of examples via alkyation or Mitsonobu coupling, as exemplified by the preparation of the below examples.

### Procedure 1

### Examples 19, 20 and 21 (racemate, enantiomer 1 and enantiomer 2 respectively), 1-(4-bromo-2,6-difluorobenzyl)-6-fluoro-3-[6-methoxy-5-(methylsulfonyl)pyridin-3-yl]-1,3,4,5-tetrahydro-2H-1-benzazepin-2-one

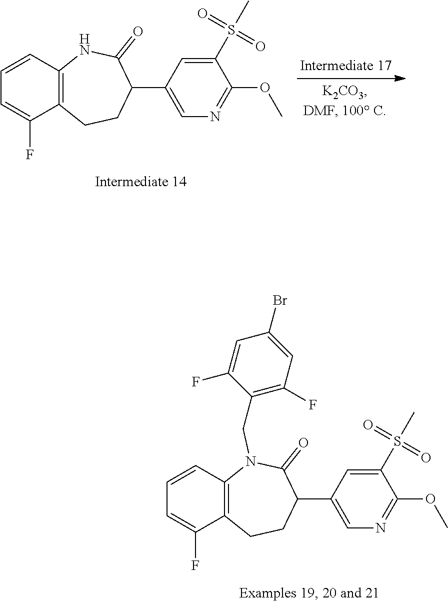 [SMILES: COc1ncc(C2CCc3c(F)cccc3NC2=O)cc1S(C)(=O)=O ; COc1ncc(C2CCc3c(F)cccc3N(Cc3c(F)cc(Br)cc3F)C2=O)cc1S(C)(=O)=O]

A mixture of 6-fluoro-3-[6-methoxy-5-(methylsulfonyl)pyridin-3-yl]-1,3,4,5-tetrahydro-2H-1-benzazepin-2-one (Intermediate 14, 210 mg, 0.58 mmol), K₂CO₃ (240 mg, 1.74 mmol) and 5-bromo-2-(bromomethyl)-1,3-difluorobenzene (Intermediate 17, 0.10 mL, 0.70 mmol) in DMF (4 mL) was heated at 100° C. overnight before cooling to rt and concentration in vacuo. The residue was partitioned between EtOAc and water and the aqueous phase was extracted with EtOAc, washed with brine and concentrated in vacuo. Purification by gradient flash column chromatography eluting with 10-60% EtOAc in 40-60° C. petroleum ether yielded the racemic title compound (Example 19,283 mg, 0.50 mmol).

### Data in Table 2

Example 19 (283 mg, 0.50 mmol) was dissolved to 20 mg/mL in MeOH:DCM (1:1) and purified by chiral preparative SFC using Method A to yield Examples 20 (100 mg, 0.18 mmol) and 21 (107 mg, 0.19 mmol) as white solids; named as enantiomer 1 (first eluting compound) and enantiomer 2 (second eluting compound) respectively based on their elution time from the column. Chiral purity analysis was performed using Method I.

### Data in Table 2

### Examples 35 and 36 (racemate and enantiomer 1 respectively), 1-(4-bromo-2,6-difluorobenzyl)-3-[5-(1,1-difluoroethyl)-6-methylpyridin-3-yl]-6-fluoro-1,3,4,5-tetrahydro-2H-1-benzazepin-2-one

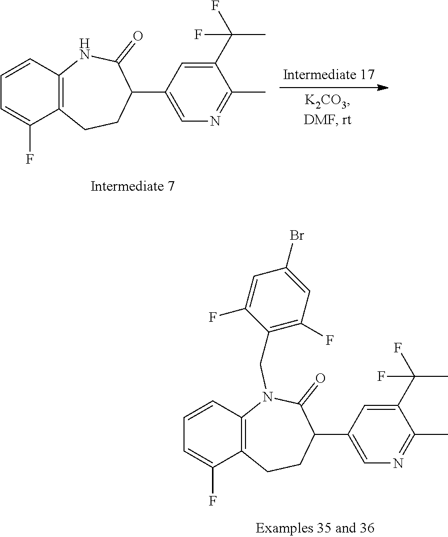 [SMILES: Cc1ncc(C2CCc3c(F)cccc3NC2=O)cc1C(C)(F)F ; Cc1ncc(C2CCc3c(F)cccc3N(Cc3c(F)cc(Br)cc3F)C2=O)cc1C(C)(F)F]

The racemic title compound (Example 35, 120 mg, 0.22 mmol) was prepared from 3-[5-(1,1-difluoroethyl)-6-methylpyridin-3-yl]-6-fluoro-1,3,4,5-tetrahydro-2H-1-benzazepin-2-one (Intermediate 7, 400 mg, 1.20 mmol), K₂CO₃ (330 mg, 2.39 mmol) and 5-bromo-2-(bromomethyl)-1,3-difluorobenzene (Intermediate 17, 424 mg, 1.48 mmol) in DMF (20 mL) at rt for 16 h using the methods of Example 19.

### Data in Table 2

Example 35 (99 mg, 0.18 mmol) was dissolved to 25 mg/mL in MeOH and purified by chiral preparative SFC using Method B to yield Example 36 (41 mg, 0.08 mmol) as an off white solid, as the first eluting compound from the column. Chiral purity analysis was performed using Method J.

### Data in Table 2

### Examples 11 and 12 (racemate and enantiomer 1 respectively), 1-(4-bromo-2,6-difluorobenzyl)-3-[5-(1,1-difluoroethyl)-6-methoxypyridin-3-yl]-6-fluoro-1,3,4,5-tetrahydro-2H-1-benzazepin-2-one

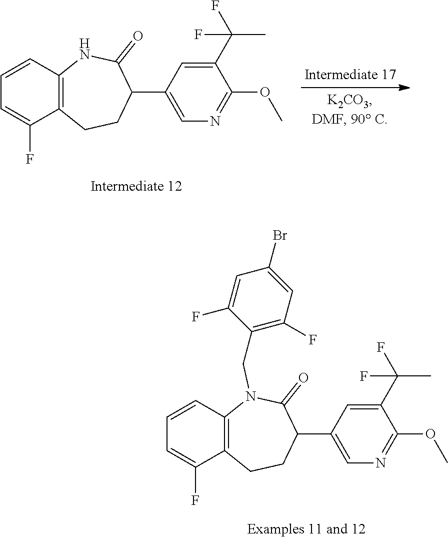 [SMILES: COc1ncc(C2CCc3c(F)cccc3NC2=O)cc1C(C)(F)F ; COc1ncc(C2CCc3c(F)cccc3N(Cc3c(F)cc(Br)cc3F)C2=O)cc1C(C)(F)F]

The racemic title compound (Example 11, 68 mg, 0.12 mmol) was prepared from 3-[5-(1,1-difluoroethyl)-6-methoxypyridin-3-yl]-6-fluoro-1,3,4,5-tetrahydro-2H-1-benzazepin-2-one (Intermediate 12, 113 mg, 0.32 mmol), K₂CO₃ (131 mg, 0.95 mmol) and 5-bromo-2-(bromomethyl)-1,3-difluorobenzene (Intermediate 17, 68 μL, 0.47 mmol) in DMF (10 mL) at 90° C. overnight using the methods of Example 19.

### Data in Table 2

Example 11 (52 mg, 0.09 mmol) was dissolved to 10 mg/mL in MeOH and purified by chiral preparative SFC using Method C to yield Example 12 (23.6 mg, 0.04 mmol) as a white solid, as the first eluting compound from the column. Chiral purity analysis was performed using Method K.

### Data in Table 2

### Example 13 (enantiomer 1), 3-({3-[5-(1,1-difluoroethyl)-6-methoxypyridin-3-yl]-6-fluoro-2-oxo-2,3,4,5-tetrahydro-1H-1-benzazepin-1-yl}methyl)-4-fluorobenzonitrile

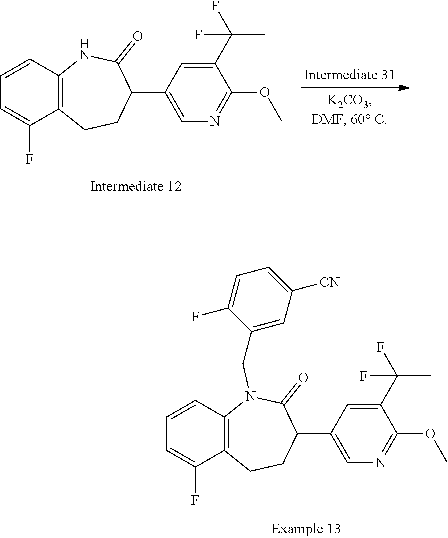 [SMILES: COc1ncc(C2CCc3c(F)cccc3NC2=O)cc1C(C)(F)F ; COc1ncc(C2CCc3c(F)cccc3N(Cc3cc(C#N)ccc3F)C2=O)cc1C(C)(F)F]

Racemic 3-({3-[5-(1,1-difluoroethyl)-6-methoxypyridin-3-yl]-6-fluoro-2-oxo-2,3,4,5-tetrahydro-1H-1-benzazepin-1-yl}methyl)-4-fluorobenzonitrile (365 mg, 0.75 mmol) was prepared from 3-[5-(1,1-difluoroethyl)-6-methoxypyridin-3-yl]-6-fluoro-1,3,4,5-tetrahydro-2H-1-benzazepin-2-one (Intermediate 12, 300 mg, 0.86 mmol), K₂CO₃ (355 mg, 2.57 mmol) and 3-(bromomethyl)-4-fluorobenzonitrile (Intermediate 31, 220 mg, 1.03 mmol) in DMF (5 mL) at 60° C. for 2 h using the methods of Example 19.

Racemic 3-({3-[5-(1,1-difluoroethyl)-6-methoxypyridin-3-yl]-6-fluoro-2-oxo-2,3,4,5-tetrahydro-1H-1-benzazepin-1-yl}methyl)-4-fluorobenzonitrile (365 mg, 0.75 mmol) was dissolved to 36 mg/mL in MeOH and purified by chiral preparative SFC using Method D to yield the title compound, (Example 13, 164 mg, 0.34 mmol) as a white solid, as the first eluting compound from the column. Chiral purity analysis was performed using Method L.

### Data in Table 2

### Example 22 (enantiomer 1), 1-(4-chloro-2,6-difluorobenzyl)-6-fluoro-3-[6-methoxy-5-(methylsulfonyl)pyridin-3-yl]-1,3,4,5-tetrahydro-2H-1-benzazepin-2-one

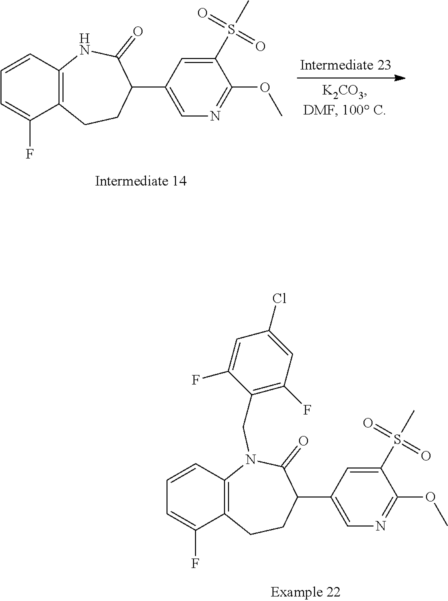 [SMILES: COc1ncc(C2CCc3c(F)cccc3NC2=O)cc1S(C)(=O)=O ; COc1ncc(C2CCc3c(F)cccc3N(Cc3c(F)cc(Cl)cc3F)C2=O)cc1S(C)(=O)=O]

Racemic 1-(4-chloro-2,6-difluorobenzyl)-6-fluoro-3-[6-methoxy-5-(methylsulfonyl)pyridin-3-yl]-1,3,4,5-tetrahydro-2H-1-benzazepin-2-one (503 mg, 0.96 mmol) was prepared from 6-fluoro-3-[6-methoxy-5-(methylsulfonyl)pyridin-3-yl]-1,3,4,5-tetrahydro-2H-1-benzazepin-2-one (Intermediate 14, 300 mg, 0.82 mmol), K₂CO₃ (400 mg, 2.89 mmol) and 2-(bromomethyl)-5-chloro-1,3-difluorobenzene (Intermediate 23, 0.14 mL, 0.99 mmol) in DMF (5 mL) at 100° C. overnight using the methods of Example 19.

Racemic 1-(4-chloro-2,6-difluorobenzyl)-6-fluoro-3-[6-methoxy-5-(methylsulfonyl)pyridin-3-yl]-1,3,4,5-tetrahydro-2H-1-benzazepin-2-one (300 mg, 0.96 mmol) was dissolved to 60 mg/mL in DMSO and purified by chiral preparative SFC using Method E to yield the title compound (Example 22, 132 mg, 0.25 mmol) as a white solid, as the first eluting compound from the column. Chiral purity analysis was performed using Method M.

### Data in Table 2

### Examples 38 and 39 (racemate and enantiomer 1 respectively), 1-(4-bromo-2,6-difluorobenzyl)-6-fluoro-3-[6-methyl-5-(methylsulfonyl)pyridin-3-yl]-1,3,4,5-tetrahydro-2H-1-benzazepin-2-one

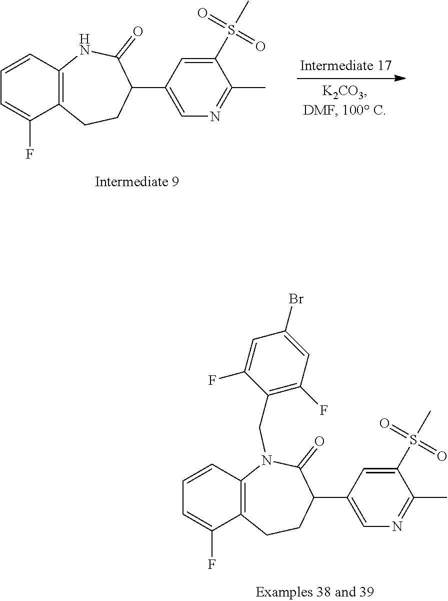 [SMILES: Cc1ncc(C2CCc3c(F)cccc3NC2=O)cc1S(C)(=O)=O ; Cc1ncc(C2CCc3c(F)cccc3N(Cc3c(F)cc(Br)cc3F)C2=O)cc1S(C)(=O)=O]

The racemic title compound (Example 38, 531 mg, 0.96 mmol) was prepared from 6-fluoro-3-[6-methyl-5-(methylsulfonyl)pyridin-3-yl]-1,3,4,5-tetrahydro-2H-1-benzazepin-2-one (Intermediate 9, 394 mg, 1.13 mmol), K₂CO₃ (469 mg, 3.39 mmol) and 5-bromo-2-(bromomethyl)-1,3-difluorobenzene (Intermediate 17, 0.19 mL, 1.32 mmol) in DMF (10 mL) at 100° C. overnight using the methods of Example 19.

### Data in Table 2

Example 38 (531 mg, 0.96 mmol) was dissolved to 50 mg/mL in MeOH/DCM (1:1) and purified by chiral preparative SFC using Method F to yield Example 39 (248 mg, 0.45 mmol) as a white solid, as the first eluting compound from the column. Chiral purity analysis was performed using Method N.

### Data in Table 2

### Example 40 (enantiomer 1), 3-[5-(1,1-difluoroethyl)-6-methylpyridin-3-yl]-1-[2,6-difluoro-4-(methoxymethyl)benzyl]-6-fluoro-1,3,4,5-tetrahydro-2H-1-benzazepin-2-one

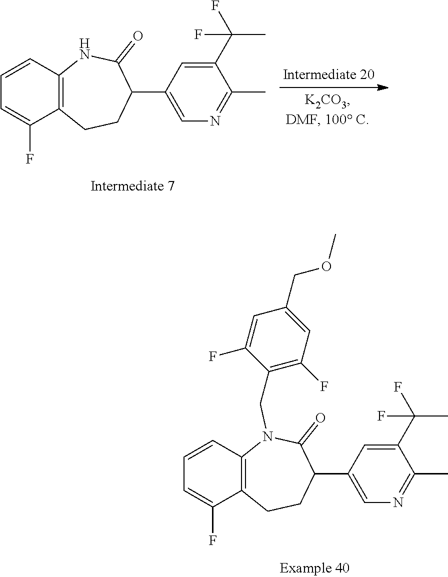 [SMILES: Cc1ncc(C2CCc3c(F)cccc3NC2=O)cc1C(C)(F)F ; COCc1cc(F)c(CN2C(=O)C(c3cnc(C)c(C(C)(F)F)c3)CCc3c(F)cccc32)c(F)c1]

The racemic title compound was prepared from 3-[5-(1,1-difluoroethyl)-6-methylpyridin-3-yl]-6-fluoro-1,3,4,5-tetrahydro-2H-1-benzazepin-2-one (Intermediate 7, 280 mg, 0.84 mmol), K₂CO₃ (415 mg, 3.00 mmol) and 4-(bromomethyl)-3,5-difluorobenzyl methyl ether (Intermediate 20, 252 mg, 1.00 mmol) in DMF (6 mL) at 100° C. overnight using the methods of Example 19.

### Data in Table 2

Racemic 3-[5-(1,1-difluoroethyl)-6-methylpyridin-3-yl]-1-[2,6-difluoro-4-(methoxymethyl)benzyl]-6-fluoro-1,3,4,5-tetrahydro-2H-1-benzazepin-2-one (302 mg, 0.59 mmol) was dissolved to 50 mg/mL in MeOH and purified by chiral preparative SFC using Method G to yield Example 40 (113 mg, 0.22 mmol) as a yellow solid, as the first eluting compound from the column. Chiral purity analysis was performed using Method O.

### Data in Table 2

### Example 41 (enantiomer 1), 3-({3-[5-(1,1-difluoroethyl)-6-methylpyridin-3-yl]-6-fluoro-2-oxo-2,3,4,5-tetrahydro-1H-1-benzazepin-1-yl}methyl)-4-fluorobenzonitrile

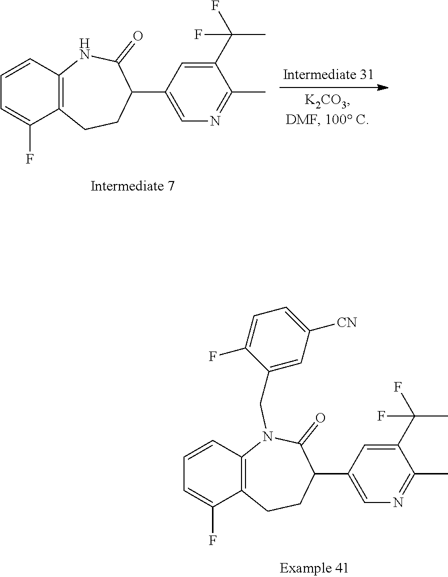 [SMILES: Cc1ncc(C2CCc3c(F)cccc3NC2=O)cc1C(C)(F)F ; Cc1ncc(C2CCc3c(F)cccc3N(Cc3cc(C#N)ccc3F)C2=O)cc1C(C)(F)F]

The racemic title compound was prepared from 3-[5-(1,1-difluoroethyl)-6-methylpyridin-3-yl]-6-fluoro-1,3,4,5-tetrahydro-2H-1-benzazepin-2-one (Intermediate 7, 280 mg, 0.84 mmol), K₂CO₃ (415 mg, 3.00 mmol) and 3-(bromomethyl)-4-fluorobenzonitrile (Intermediate 31, 214 mg, 1.00 mmol) in DMF (6 mL) at 100° C. overnight using the methods of Example 19.

### Data in Table 2

Racemic 3-({3-[5-(1,1-difluoroethyl)-6-methylpyridin-3-yl]-6-fluoro-2-oxo-2,3,4,5-tetrahydro-1H-1-benzazepin-1-yl}methyl)-4-fluorobenzonitrile (328 mg, 0.70 mmol) was dissolved to 50 mg/mL in MeOH and purified by chiral preparative SFC using Method H to yield Example 41 (136 mg, 0.29 mmol) as a yellow oil, as the first eluting compound from the column. Chiral purity analysis was performed using Method P.

### Data in Table 2

### Procedure 2

### Example 1, 1-(4-bromo-2,6-difluorobenzyl)-3-[3-(1,1-difluoroethyl)-4-methylphenyl]-6-fluoro-1,3,4,5-tetrahydro-2H-1,3-benzodiazepin-2-one

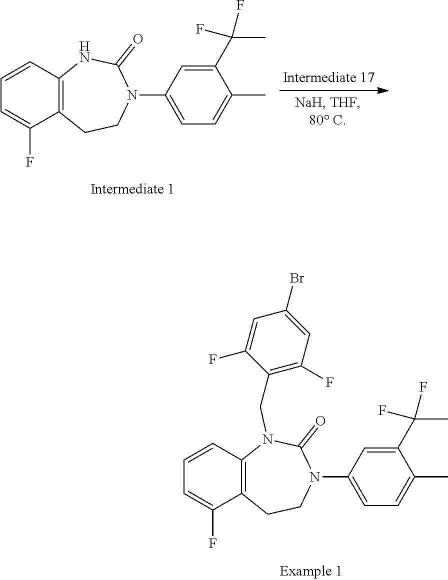 [SMILES: Cc1ccc(N2CCc3c(F)cccc3NC2=O)cc1C(C)(F)F ; Cc1ccc(N2CCc3c(F)cccc3N(Cc3c(F)cc(Br)cc3F)C2=O)cc1C(C)(F)F]

3-[3-(1,1-Difluoroethyl)-4-methylphenyl]-6-fluoro-1,3,4,5-tetrahydro-2H-1,3-benzodiazepin-2-one (Intermediate 1, 0.16 g, 0.48 mmol) was dissolved in THF (5 mL), NaH (60% dispersion in mineral oil, 0.23 g, 5.75 mmol) was added at rt and the reaction mixture was stirred at 80° C. for 90 min before cooling to 0° C. 5-Bromo-2-(bromomethyl)-1,3-difluorobenzene (Intermediate 17, 0.14 g, 0.49 mmol) was added and the reaction mixture was stirred at 80° C. for 2 h before partitioning between saturated aqueous NH₄Cl (30 mL) and EtOAc (50 mL). The phases were separated and the aqueous layer was extracted with EtOAc (3×30 mL). The combined organic layers were dried (Na₂SO₄) and concentrated in vacuo. Purification by gradient flash column chromatography eluting with 0-30% EtOAc in hexane yielded the title compound (75 mg, 0.14 mmol) as an off-white powder.

### Data in Table 2

### Example 23, 1-(4-bromo-2,6-difluorobenzyl)-3-[5-(1,1-difluoroethyl)-2,6-dimethoxypyridin-3-yl]-6-fluoro-1,3,4,5-tetrahydro-2H-1,3-benzodiazepin-2-one

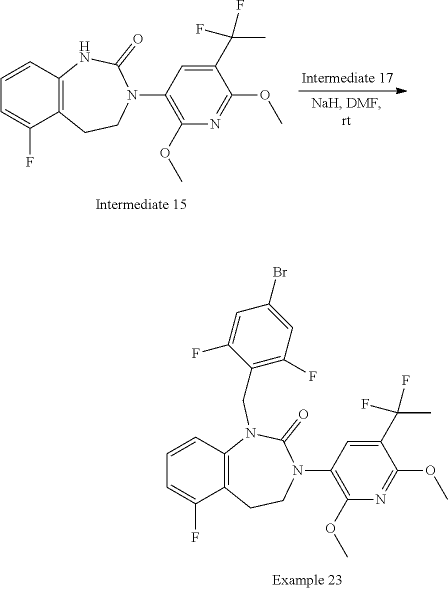 [SMILES: COc1nc(OC)c(C(C)(F)F)cc1N1CCc2c(F)cccc2NC1=O ; COc1nc(OC)c(C(C)(F)F)cc1N1CCc2c(F)cccc2N(Cc2c(F)cc(Br)cc2F)C1=O]

A mixture of 3-[5-(1,1-difluoroethyl)-2,6-dimethoxypyridin-3-yl]-6-fluoro-1,3,4,5-tetrahydro-2H-1,3-benzodiazepin-2-one (Intermediate 15, 110 mg, 0.29 mmol), 5-bromo-2-(bromomethyl)-1,3-difluoro-benzene (Intermediate 17, 98.9 mg, 0.35 mmol) and NaH (13.8 mg, 0.58 mmol) in DMF (11 mL) was stirred at rt for 35 min. Water (20 mL) was added and the mixture stirred for 30 min before the title compound (80 mg, 0.14 mmol) was isolated as a white solid after filtration and washing with water (10 mL) and hexane (10 mL).

### Data in Table 2

### Procedure 3

### Example 26, 1-(4-bromo-2,6-difluorobenzyl)-6-fluoro-3-[4-methyl-3-(methylsulfanyl)phenyl]-1,3,4,5-tetrahydro-2H-1,3-benzodiazepin-2-one

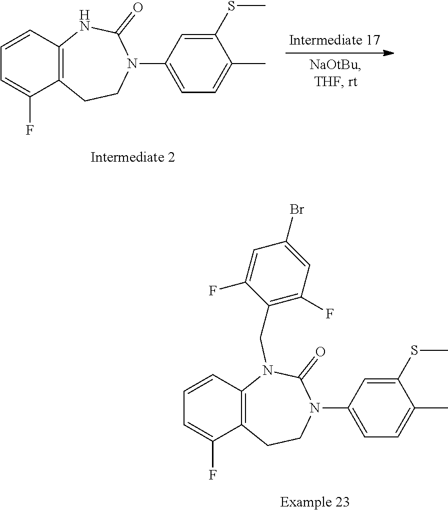 [SMILES: CSc1cc(N2CCc3c(F)cccc3NC2=O)ccc1C ; CSc1cc(N2CCc3c(F)cccc3N(Cc3c(F)cc(Br)cc3F)C2=O)ccc1C]

Sodium tert-butoxide (0.12 g, 1.25 mmol) was added to a solution of 6-fluoro-3-[4-methyl-3-(methylsulfanyl)phenyl]-1,3,4,5-tetrahydro-2H-1,3-benzodiazepin-2-one (Intermediate 2, 0.20 g, 0.63 mmol) in THF (20 mL) at rt. After cooling to 0° C., 5-bromo-2-(bromomethyl)-1,3-difluorobenzene (Intermediate 17, 0.19 g, 0.66 mmol) was added and the reaction mixture was stirred at rt for 4 h. Ice cold water (30 mL) was added and the mixture was extracted with EtOAc (2×30 mL). The combined organic phases were dried (Na₂SO₄), filtered, and concentrated in vacuo. Purification by gradient flash column chromatography eluting with 40-50% EtOAc in 40-60° C. petroleum ether yielded the title compound (110 mg, 0.21 mmol).

### Data in Table 2

### Procedure 4

### Example 25, 1-(2,6-difluoro-4-methoxybenzyl)-3-[2,6-dimethoxy-5-(methylsulfonyl)pyridin-3-yl]-6-fluoro-1,3,4,5-tetrahydro-2H-1,3-benzodiazepin-2-one

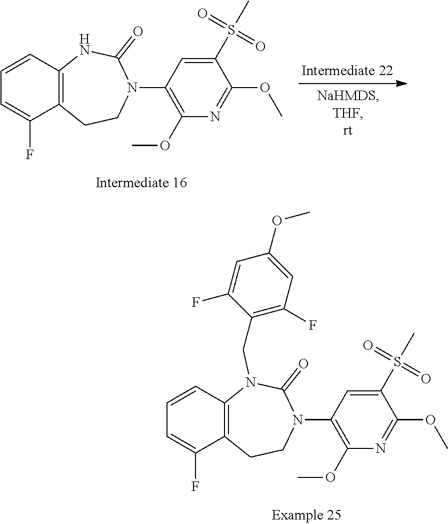 [SMILES: COc1nc(OC)c(S(C)(=O)=O)cc1N1CCc2c(F)cccc2NC1=O ; COc1cc(F)c(CN2C(=O)N(c3cc(S(C)(=O)=O)c(OC)nc3OC)CCc3c(F)cccc32)c(F)c1]

NaHMDS (1M in THF, 0.15 mL, 0.15 mmol) was added to a solution of 3-[2,6-dimethoxy-5-(methylsulfonyl)pyridin-3-yl]-6-fluoro-1,3,4,5-tetrahydro-2H-1,3-benzodiazepin-2-one Intermediate 16, 50 mg, 0.13 mmol) in THF (1 mL) at rt and the mixture stirred at rt for 15 min. A solution of 4-(bromomethyl)-3,5-difluorophenyl methyl ether (Intermediate 22, 36 mg, 0.15 mmol) in THF (0.5 mL) was then added and the reaction mixture was stirred at rt for 3 h before the addition of H₂O and EtOAc. The phases were separated, the aqueous phase was extracted with EtOAc, and the combined organic phases were washed with H₂O and brine and concentrated in vacuo. Purification by gradient column chromatography, eluting with 0-100% EtOAc in petroleum ether yielded the title compound (51 mg, 0.09 mmol).

### Data in Table 2

### Procedure 5

### Example 7, 3-[5-(1,1-difluoroethyl)-6-methoxypyridin-3-yl]-1-[2,6-difluoro-4-(methoxymethyl)benzyl]-6-fluoro-1,3,4,5-tetrahydro-2H-1,3-benzodiazepin-2-one

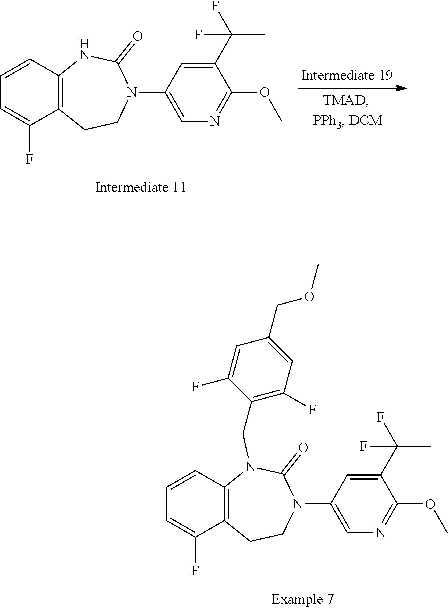 [SMILES: COc1ncc(N2CCc3c(F)cccc3NC2=O)cc1C(C)(F)F ; COCc1cc(F)c(CN2C(=O)N(c3cnc(OC)c(C(C)(F)F)c3)CCc3c(F)cccc32)c(F)c1]

A mixture of TMAD (52 mg, 0.30 mmol), triphenylphosphine (9 mg, 0.30 mmol), 3-[5-(1,1-difluoroethyl)-6-methoxypyridin-3-yl]-6-fluoro-1,3,4,5-tetrahydro-2H-1,3-benzodiazepin-2-one (Intermediate 11, 53 mg, 0.15 mmol) and [2,6-difluoro-4-(methoxymethyl)phenyl]methanol (Intermediate 19, 31 mg, 0.16 mmol) in DCM (2 mL) was stirred at rt for 3 d. Further DCM (5 mL) was added and the reaction mixture was stirred at rt overnight before the addition of further DCM (5 mL) and aqueous HCl (1M, 5 mL). The phases were separated and the aqueous layer was extracted with DCM (2×5 mL). The combined organic phases were concentrated in vacuo, and purification by gradient flash column chromatography, eluting with 10-60% EtOAc in 40-60° C. petroleum ether, followed by preparative reversed phase HPLC (Phenomenex Gemini-NX 5 μm C18 column, 100×30 mm, eluting with 60 to 100% MeCN/Solvent B over 12.5 min at 30 mL/min [where solvent B is 0.2% of (28% NH₃/H₂O) in H₂O] and collecting fractions by monitoring at 205 nm) yielded the title compound (5 mg, 0.01 mmol) as a clear glassy solid.

### Data in Table 2

Further examples prepared by the above procedures are detailed in Table 2.

TABLE 2

| Ex. | | Stereochemistry | Intermediates/ | | LCMS | Chiral purity |
|---|---|---|---|---|---|---|
| No. | Name | Comment | Procedure | ¹H NMR | (Method 1) | analysis |
| 1 | 1-(4-bromo-2,6- | Achiral | Intermediates | (400 MHz, DMSO-d₆) δ: ppm | m/z 539.0, 541.0 | — |
| | difluorobenzyl)-3-[3-(1,1- | | 1 and 17 | 1.91 (t, J = 19.1, 3H), 2.34 (s, 3H), | (M + H)+ (ES+), at | |
| | difluoroethyl)-4- | | Procedure 2 | 2.96 (t, J = 6.4, 2H), 3.76 (t, J = 6.6 | 6.57 min, 100% | |
| | methylphenyl]-6-fluoro- | | | 2H), 5.06 (s, 2H), 7.02-7.10 (m, | | |
| | 1,3,4,5-tetrahydro-2H-1,3- | | | 3H), 7.18 (d, J = 7.9, 1H), 7.30- | | |
| | benzodiazepin-2-one | | | 7.38 (m, 2H), 7.43 (d, J = 7.3, 2H) | | |
| 2 | 1-(4-bromo-2,6- | Achiral | Intermediates | (400 MHz, DMSO-d₆) δ: ppm | m/z 559.0, 561.0 | — |
| | difluorobenzyl)-3-{5-(1,1- | | 10 and 17 | 1.96 (t, J = 19.3, 3H), 3.00 (t, | (M + H)+ (ES+), at | |
| | difluoroethyl)-6- | | Procedure 1 | J = 6.3, 2H), 3.78 (t, J = 6.3, 2H), | 6.17 min, 100% | |
| | [(²H₃)methyloxy]pyridin-3- | | | 5.04 (s, 2H), 6.97-7.15 (m, 1H), | | |
| | yl}-6-fluoro-1,3,4,5- | | | 7.31-7.36 (m, 2H), 7.42 (d, J = 7.4, | | |
| | tetrahydro-2H-1,3- | | | 2H), 7.59 (d, J = 2.3, 1H), 8.02 (d, | | |
| | benzodiazepin-2-one | | | J = 2.3, 1H) | | |
| 3 | 1-(4-bromo-2,6- | Achiral | Intermediates | (400 MHz, DMSO-d₆) δ: ppm | m/z 556.1, 558.1 | — |
| | difluorobenzyl)-3-[5-(1,1- | | 11 and 17 | 1.96 (t, J = 19.1, 3H), 3.00 (t, | (M + H)+ (ES+), at | |
| | difluoroethyl)-6- | | Procedure 2 | J = 6.3, 2H), 3.78 (t, J = 6.3, 2H), | 6.12 min, 100% | |
| | methoxypyridin-3-yl]-6- | | | 3.90 (s, 3H), 5.04 (s, 2H), 7.03- | | |
| | fluoro-1,3,4,5-tetrahydro- | | | 7.12 (m, 1H), 7.31-7.37 (m, 2H), | | |
| | 2H-1,3-benzodiazepin-2-one | | | 7.42 (d, J = 7.4, 2H), 7.59 (d, | | |
| | | | | J = 2.3, 1H), 8.03 (d, J = 2.0, 1H) | | |
| 4 | 3-[5-(1,1-difluoroethyl)-6- | Achiral | Intermediates | (400 MHz, CDCl₃) δ: ppm 1.96 (t, | m/z 490.1 | — |
| | methoxypyridin-3-yl]-6- | | 8 and 17 | J = 19.0, 3H), 3.08 (t, J = 6.4, 2H), | (M + H)+ (ES+), at | |
| | fluoro-1-(2-fluoro-4- | | Procedure 1 | 3.75 (s, 3H), 3.78 (d, J = 6.3, 2H), | 5.72 min, 97% | |
| | methoxybenzyl)-1,3,4,5- | | | 3.95 (s, 3H), 4.99 (s, 2H), 6.49- | | |
| | tetrahydro-2H-1,3- | | | 6.63 (m, 2H), 6.89 (t, J = 8.6, 1H), | | |
| | benzodiazepin-2-one | | | 7.05 (d, J = 8.2, 1H), 7.16-7.30 (m, | | |
| | | | | 2H), 7.59 (d, J = 3.2, 1H), 7.96 (d, | | |
| | | | | J = 2.8, 1H) | | |
| 5 | 3-[5-(1,1-difluoroethyl)-6- | Achiral | Intermediates | (400 MHz, CDCl₃) δ: ppm 1.98 (t, | m/z 525.2 | — |
| | methoxypyridin-3-yl]-1- | | 11, and 28 | J = 18.7, 3H), 3.12 (t, J = 6.4, 2H), | (M + H)+ (ES+), at | |
| | (2,6-difluoro-4- | | Procedure 1 | 3.79 (t, J = 6.4, 2H), 3.97 (s, 3H), | 5.68 min, 100% | |
| | {[(²H₃)methyloxy]methyl} | | | 4.36 (s, 2H), 5.14 (s, 2H), 6.80 (d, | | |
| | benzyl)-6-fluoro-1,3,4,5- | | | J = 8.2, 2H), 6.88 (t, J = 8.6, 1H), | | |
| | tetrahydro-2H-1,3- | | | 7.13 (d, J = 8.1, 1H), 7.18-7.26 (m, | | |
| | benzodiazepin-2-one | | | 1H), 7.62 (d, J = 2.3, 1H), 7.99 (d, | | |
| | | | | J = 2.3, 1H) | | |
| 6 | 3-[5-(1,1-difluoroethyl)-6- | Achiral | Intermediates | (400 MHz, CDCl₃) δ: ppm 1.99 (t, | m/z 511.1 | — |
| | methoxypyridin-3-yl]-1- | | 11 and 29 | J = 18.8, 3H), 3.10 (t, J = 6.5, 2H), | (M + H)+ (ES+), at | |
| | {2,6-difluoro-4- | | Procedure 1 | 3.78 (t, J = 6.5, 2H), 3.96 (s, 3H), | 5.99 min, 100% | |
| | [(²H₃)methyloxy]benzyl}-6- | | | 5.07 (s, 2H), 6.35 (d, J = 9.5, 2H), | | |
| | fluoro-1,3,4,5-tetrahydro- | | | 6.87 (t, J = 8.5, 1H), 7.11 (d, J = 8.1, | | |
| | 2H-1,3-benzodiazepin-2-one | | | 1H), 7.20 (dd, J = 8.3, 6.2, 1H), | | |
| | | | | 7.57-7.63 (m, 1H), 7.98 (d, J = 2.8, | | |
| | | | | 1H) | | |
| 7 | 3-[5-(1,1-difluoroethyl)-6- | Achiral | Intermediates | (400 MHz, METHANOL-d₄) δ: | m/z 522.2 | — |
| | methoxypyridin-3-yl]-1- | | 11 and 19 | ppm 1.95 (t, J = 18.7, 3H), 3.11 (t, | (M + H)+ (ES+), at | |
| | [2,6-difluoro-4- | | Procedure 5 | J = 6.2, 2H), 3.35 (s, 3H), 3.80 (t, | 5.79 min, 100% | |
| | (methoxymethyl)benzyl]-6- | | | J = 6.4, 2H), 3.88-4.04 (m, 3H) | | |
| | fluoro-1,3,4,5-tetrahydro- | | | 4.38 (s, 2H), 5.17 (s, 2H), 6.88 (d, | | |
| | 2H-1,3-benzodiazepin-2-one | | | J = 8.2 Hz, 2H), 6.92-7.00 (m, 1H) | | |
| | | | | 7.29-7.32 (m, 2H), 7.63 (d, J = 2.4, | | |
| | | | | 1H), 7.97 (d, J = 2.4, 1H) | | |
| 8 | 3-[5-(1,1-difluoroethyl)-6- | Achiral | Intermediates | (400 MHz, CDCl₃) δ: ppm 1.95 (t, | m/z 508.1 | — |
| | methoxypyridin-3-yl]-1- | | 11 and 22 | J = 18.7, 3H), 3.10 (t, J = 6.4, 2H), | (M + H)+ (ES+), at | |
| | (2,6-difluoro-4- | | Procedure 1 | 3.73 (s, 3H), 3.77 (t, J = 6.4, 2H), | 5.81 min, 100% | |
| | methoxybenzyl)-6-fluoro- | | | 3.96 (s, 3H), 5.06 (s, 2H), 6.31- | | |
| | 1,3,4,5-tetrahydro-2H-1,3- | | | 6.40 (m, 2H), 6.87 (t, J = 8.5, 1H), | | |
| | benzodiazepin-2-one | | | 7.11 (d, J = 8.2, 1H), 7.16-7.28 (m, | | |
| | | | | 1H), 7.60 (d, J = 2.4, 1H), 7.98 (d, | | |
| | | | | J = 2.8, 1H) | | |
| 9 | 1-(4-chloro-2,6- | Achiral | Intermediates | (400 MHz, DMSO-d₆) δ: ppm | m/z 512.1, 514.1 | — |
| | difluorobenzyl)-3-[5-(1,1 | | 11 and 23 | 1.96 (t, J = 19.1, 3H), 3.00 (t, | (M + H)+ (ES+), at | |
| | difluoroethyl)-6- | | Procedure 1 | J = 6.6, 2H), 3.78 (t, J = 6.6, 2H) | 7.14 min, 100% | |
| | methoxypyridin-3-yl]-6- | | | 3.90 (s, 3H), 5.06 (s, 2H), 7.03- | | |
| | fluoro-1,3,4,5-tetrahydro- | | | 7.09 (m, 1H), 7.23-7.49 (m, 4H), | | |
| | 2H-1,3-benzodiazepin-2-one | | | 7.59 (d, J = 2.4, 1H), 8.03 (d, | | |
| | | | | J = 2.4, 1H) | | |
| 10 | 1-(4-bromo-2-fluorobenzyl)- | Achiral | Intermediates | (400 MHz, CDCl₃) δ: ppm 1.97 (t, | m/z 538.0, 540.0 | — |
| | 3-[5-(1,1-difluoroethyl)-6- | | 11 and 26 | J = 18.8, 3H), 3.12 (t, J = 6.2, 2H), | (M + H)+ (ES+), at | |
| | methoxypyridin-3-yl]-6- | | Procedure 1 | 3.79 (d, J = 6.4, 2H), 3.96 (s, 3H), | 6.38 min, 96% | |
| | fluoro-1,3,4,5-tetrahydro- | | | 4.99 (s, 2H), 6.92 (td, J = 8.5, 1.0, | | |
| | 2H-1,3-benzodiazepin-2-one | | | 1H), 7.02 (d, J = 8.1, 1H), 7.15- | | |
| | | | | 7.32 (m, 4H), 7.60 (d, J = 2.8, 1H), | | |
| | | | | 7.97 (d, J = 2.6, 1H) | | |
| 11 | 1-(4-bromo-2,6- | Racemic | Intermediates | (400 MHz, DMSO-d₆) δ: ppm | m/z 555.1, 557.1 | — |
| | difluorobenzyl)-3-[5-(1,1- | | 12 and 17 | 1.97 (t, J = 19.1, 3H), 2.06-2.18 | (M + H)+ (ES+), at | |
| | difluoroethyl)-6- | | Procedure 1 | (m, 1H), 2.30-2.47 (m, 2H), 2.97 | 6.49 min, 100% | |
| | methoxypyridin-3-yl]-6- | | | (dd, J = 13.0, 6.4, 1H), 3.67 (dd, | | |
| | fluoro-1,3,4,5-tetrahydro- | | | J = 12.0, 7.3, 1H), 3.90 (s, 3H), | | |
| | 2H-1-benzazepin-2-one | | | 4.71 (d, J = 14.9, 1H), 5.43 (d, | | |
| | | | | J = 14.8, 1H), 7.13 (ddd, J = 9.3, 6.8, | | |
| | | | | 2.5, 1H), 7.35-7.44 (m, 4H), 7.82 | | |
| | | | | (d, J = 2.3, 1H), 8.09 (d, J = 2.2, | | |
| | | | | 1H) | | |
| 12 | 1-(4-bromo-2,6- | Enantiomer 1 | Intermediates | (400 MHz, DMSO-d₆) δ: ppm | m/z 555.0, 557.0 | Chiral purity |
| | difluorobenzyl)-3-[5-(1,1- | | 12 and 17 | 1.97 (t, J = 19.1, 3H), 2.06-2.20 | (M + H)+ (ES+), at | analysis: 1.29 |
| | difluoroethyl)-6- | | Procedure 1, | (m, 1H), 2.24-2.44 (m, 2H), 2.90- | 6.41 min, 100% | min, 100% |
| | methoxypyridin-3-yl]-6- | | then chiral | 3.04 (m, 1H), 3.58-3.77 (m, 1H) | | (Method K) |
| | fluoro-1,3,4,5-tetrahydro- | | separation | 3.90 (s, 3H), 4.71 (d, J = 14.5, 1H), | | |
| | 2H-1-benzazepin-2-one | | | 5.43 (d, J = 14.8, 1H), 7.13 (t, | | |
| | | | | J = 7.8, 1H), 7.33-7.47 (m, 4H), | | |
| | | | | 7.82 (s, 1H), 8.09 (s, 1H) | | |
| 13 | 3-({3-[5-(1,1-difluoroethyl)- | Enantiomer 1 | Intermediates | (400 MHz, DMSO-d₆) δ: ppm | m/z 484.2 | Chiral purity |
| | 6-methoxypyridin-3-yl]-6- | | 12 and 31 | 1.97 (t, J = 19.1, 3H), 2.11-2.25 | (M + H)+ (ES+), at | analysis: 1.22 |
| | fluoro-2-oxo-2,3,4,5- | | Procedure 1, | (m, 1H), 2.36-2.48 (m, 2H), 2.93- | 5.67 min, 100% | min, 99.8% |
| | tetrahydro-1H-1-benzazepin- | | then chiral | 3.07 (m, 1H), 3.76-3.87 (m, 1H), | | (Method L) |
| | 1-yl}methyl)-4- | | separation | 3.91 (s, 3H), 4.99 (d, J = 15.6, 1H), | | |
| | fluorobenzonitrile | | | 5.18 (d, J = 15.6, 1H), 7.15 (t, | | |
| | | | | J = 8.6, 1H), 7.31 (d, J = 7.8, 1H), | | |
| | | | | 7.36-7.47 (m, 2H), 7.77-7.87 (m, | | |
| | | | | 1H), 7.87-7.94 (m, 2H), 8.15 (s, | | |
| | | | | 1H) | | |
| 14 | 1-(4-bromo-2,6- | Achiral | Intermediates | (400 MHz, DMSO-d₆) δ: ppm | m/z 570.1, 572.1 | — |
| | difluorobenzyl)-6-fluoro-3- | | 13 and 17 | 3.01 (t, J = 6.3, 2H), 3.27 (s, 3H), | (M + H)+ (ES+), at | |
| | [6-methoxy-5- | | Procedure 2 | 3.80 (t, J = 6.3, 2H), 4.00 (s, 3H), | 5.14 min, 98% | |
| | (methylsulfonyl)pyridin-3- | | | 5.07 (s, 2H), 7.03-7.11 (m, 1H), | | |
| | yl]-1,3,4,5-tetrahydro-2H- | | | 7.31-7.41 (m, 2H), 7.42 (d, J = 7.0, | | |
| | 1,3-benzodiazepin-2-one | | | 2H), 7.89 (d, J = 2.7, 1H), 8.25 (d, | | |
| | | | | J = 2.7, 1H) | | |
| 15 | 6-fluoro-3-[6-methoxy-5- | Achiral | Intermediates | (400 MHz, DMSO-d₆) δ: ppm | m/z 540.1 | — |
| | (methylsulfonyl)pyridin-3- | | 13 and 30 | 3.00 (t, J = 6.3, 2H), 3.27 (s, 3H), | (M + H)+ (ES+), at | |
| | yl]-1-(2,3,6-trifluoro-4- | | Procedure 2 | 3.81 (t, J = 6.3, 2H), 3.84 (s, 3H), | 4.95 min, 100% | |
| | methoxybenzyl)-1,3,4,5- | | | 4.00 (s, 3H), 5.07 (s, 2H), 7.00 | | |
| | tetrahydro-2H-1,3- | | | (dd, J = 11.1, 7.2, 1H), 7.08 (t, | | |
| | benzodiazepin-2-one | | | J = 8.4, 1H), 7.31-7.41 (m, 2H), | | |
| | | | | 7.89 (d, J = 2.3, 1H), 8.25 (d, | | |
| | | | | J = 2.0, 1H) | | |
| 16 | 1-(2,6-difluoro-4- | Achiral | Intermediates | (400 MHz, DMSO-d₆) δ: ppm | m/z 522.1 | — |
| | methoxybenzyl)-6-fluoro-3- | | 13 and 22 | 2.98 (t, J = 6.2, 2H), 3.27 (s, 3H), | (M + H)+ (ES+), at | |
| | [6-methoxy-5- | | Procedure 1 | 3.73 (s, 3H), 3.80 (t, J = 6.2, 2H), | 4.79 min, 97% | |
| | (methylsulfonyl)pyridin-3- | | | 4.00 (s, 3H), 5.05 (s, 2H), 6.66 (d, | | |
| | yl]-1,3,4,5-tetrahydro-2H- | | | J = 10.2, 2H), 7.05 (t, J = 8.3, 1H), | | |
| | 1,3-benzodiazepin-2-one | | | 7.24-7.43 (m, 2H), 7.87 (d, J = 2.3, | | |
| | | | | 1H), 8.25 (d, J = 2.7, 1H) | | |
| 17 | 1-(4-bromo-2-fluoro-6- | Achiral | Intermediates | (400 MHz, CDCl₃) δ: ppm 2.44 | m/z 566.2, 568.2 | — |
| | methylbenzyl)-6-fluoro-3- | | 13 and 24 | (s, 3H), 3.08 (t, J = 6.6, 2H), 3.20 | (M + H)+ (ES+), at | |
| | [6-methoxy-5- | | Procedure 1 | (s, 3H), 3.75 (t, J = 6.5, 2H), 4.07 | 5.52 min, 95% | |
| | (methylsulfonyl)pyridin-3- | | | (s, 3H), 5.03 (s, 2H), 6.95 (t | | |
| | yl]-1,3,4,5-tetrahydro-2H- | | | J = 8.5, 1H), 7.01 (d, J = 9.8, 1H), | | |
| | 1,3-benzodiazepin-2-one | | | 7.08-7.17 (m, 2H), 7.20-7.33 (m, | | |
| | | | | 1H), 8.00 (dt, J = 2.8, 0.6, 1H), | | |
| | | | | 8.14 (dt, J = 2.7, 0.6, 1H) | | |
| 18 | 1-(2,4-difluoro-6- | Achiral | Intermediates | (400 MHz, CDCl₃) δ: ppm 2.44 | m/z 506.1 | — |
| | methylbenzyl)-6-fluoro-3- | | 13 and 25 | (s, 3H), 3.10 (t, J = 6.5, 2H), 3.20 | (M + H)+ (ES+), at | |
| | [6-methoxy-5- | | Procedure 1 | (s, 3H), 3.74 (t, J = 6.6, 2H), 4.06 | 5.10 min, 100% | |
| | (methylsulfonyl)pyridin-3- | | | (s, 3H), 5.04 (s, 2H), 6.56 (ddd, | | |
| | yl]-1,3,4,5-tetrahydro-2H- | | | J = 11.0, 8.6, 2.6, 1H), 6.67 (dd, | | |
| | 1,3-benzodiazepin-2-one | | | J = 9.1, 2.6, 1H), 6.93 (td, J = 8.5, | | |
| | | | | 1.0, 1H), 7.14 (dd, J = 8.1, 1.0, | | |
| | | | | 1H), 7.26 (td, J = 8.2, 6.2, 1H), | | |
| | | | | 8.00 (d, J = 2.8, 1H), 8.13 (d, | | |
| | | | | J = 2.7, 1H) | | |
| 19 | 1-(4-bromo-2,6- | Racemic | Intermediates | (400 MHz, CDCl₃) δ: ppm 2.13- | m/z 569.0, 571.0 | — |
| | difluorobenzyl)-6-fluoro-3- | | 14 and 17 | 2.29 (m, 1H), 2.41-2.59 (m, 2H), | (M + H)+ (ES+), at | |
| | [6-methoxy-5- | | Procedure 1 | 3.09-3.18 (m, 1H), 3.23 (s, 3H), | 5.43 min, 100% | |
| | (methylsulfonyl)pyridin-3- | | | 3.59-3.69 (m, 1H), 4.10 (s, 3H), | | |
| | yl]-1,3,4,5-tetrahydro-2H-1- | | | 4.65 (d, J = 14.6, 1H), 5.62 (d, | | |
| | benzazepin-2-one | | | J = 14.6, 1H), 6.96-7.08 (m, 3H), | | |
| | | | | 7.14 (d, J = 7.9, 1H), 7.23-7.36 (m, | | |
| | | | | 1H), 8.17 (d, J = 1.8, 1H), 8.26 (d, | | |
| | | | | J = 2.1, 1H) | | |
| 20 | 1-(4-bromo-2,6- | Enantiomer 1 | Intermediates | (400 MHz, DMSO-d₆) δ: ppm | m/z 569.0, 571.0 | Chiral purity |
| | difluorobenzyl)-6-fluoro-3- | | 14 and 17 | 2.10-2.20 (m, 1H), 2.29-2.45 (m, | (M + H)+ (ES+), at | analysis: 2.85 |
| | [6-methoxy-5- | | Procedure 1, | 2H), 2.94-3.03 (m, 1H), 3.28 (s, | 5.44 mm, 100% | min, 100% |
| | (methylsulfonyl)pyridin-3- | | then chiral | 3H), 3.74-3.83 (m, 1H), 4.01 (s, | | (Method I) |
| | yl]-1,3,4,5-tetrahydro-2H-1- | | separation | 3H), 4.72 (d, J= 14.8, 1H), 5.44 (d, | | |
| | benzazepin-2-one | | | J = 14.8, 1H), 7.13 (td, J = 7.6, 2.7, | | |
| | | | | 1H), 7.35-7.45 (m, 4H), 8.16 (d, | | |
| | | | | J = 2.4, 1H), 8.31 (d, J = 2.4, 1H) | | |
| 21 | 1-(4-bromo-2,6- | Enantiomer 2 | Intermediates | (400 MHz, DMSO-d₆) δ: ppm | m/z 569.0, 571.0 | Chiral purity |
| | difluorobenzyl)-6-fluoro-3- | | 14 and 17 | 2.10-2.20 (m, 1H), 2.29-2.45 (m, | (M + H)+ (ES+), at | analysis: 4.47 |
| | [6-methoxy-5- | | Procedure 1, | 2H), 2.94-3.03 (m, 1H), 3.28 (s, | 5.43 mm, 100% | min, 100% |
| | (methylsulfonyl)pyridin-3- | | then chiral | 3H), 3.74-3.83 (m, 1H), 4.01 (s, | | (Method I) |
| | yl]-1,3,4,5-tetrahydro-2H-1- | | separation | 3H), 4.72 (d, J= 14.8, 1H), 5.44 (d, | | |
| | benzazepin-2-one | | | J = 15.2, 1H), 7.10-7.16 (m, 1H), | | |
| | | | | 7.35-7.45 (m, 4H), 8.16 (s, 1H), | | |
| | | | | 8.31 (d, J = 2.0, 1H) | | |
| 22 | 1-(4-chloro-2,6- | Enantiomer 1 | Intermediates | (400 MHz, DMSO-d₆) δ: ppm | m/z 525.2, 527.2 | Chiral purity |
| | difluorobenzyl)-6-fluoro-3- | | 14 and 23 | 2.07-2.18 (m, 1H), 2.28-2.43 (m, | (M + H)+ (ES+), at | analysis: 1.88 |
| | [6-methoxy-5- | | Procedure 1, | 2H), 2.96 (dd, J = 12.6, 6.6, 1H), | 5.09 min, 100% | min, 100% |
| | (methylsulfonyl)pyridin-3- | | then chiral | 3.26 (s, 3H), 3.76 (dd, J = 11.7, | | (Method M) |
| | yl]-1,3,4,5-tetrahydro-2H-1- | | separation | 7.6, 1H), 3.95-4.03 (m, 3H), 4.70 | | |
| | benzazepin-2-one | | | (d, J = 14.9, 1H), 5.43 (d, J = 14.9, | | |
| | | | | 1H), 7.07-7.16 (m, 1H), 7.27 (d, | | |
| | | | | J = 7.5, 2H), 7.33-7.44 (m, 2H), | | |
| | | | | 8.14 (d, J = 2.2, 1H), 8.29 (d, | | |
| | | | | J = 2.3, 1H) | | |
| 23 | 1-(4-bromo-2,6- | Achiral | Intermediates | (400 MHz, CDCl₃) δ: ppm 1.93 (t, | m/z 586.1, 588.1 | — |
| | difluorobenzyl)-3-[5-(1,1- | | 15 and 17 | J = 18.7, 3H), 3.09 (t, J = 6.2, 2H), | (M + H)+ (ES+), at | |
| | difluoroethyl)-2,6- | | Procedure 2 | 3.68 (dd, J = 6.9, 5.7, 2H), 3.89 (s, | 6.66 min, 100% | |
| | dimethoxypyridin-3-yl]-6- | | | 3H), 3.95 (s, 3H), 5.05 (s, 2H), | | |
| | fluoro-1,3,4,5-tetrahydro- | | | 6.86 (td, J = 8.6, 1.0, 1H), 6.96- | | |
| | 2H-1,3-benzodiazepin-2-one | | | 7.04 (m, 2H), 7.06 (d, J = 8.2, 1H), | | |
| | | | | 7.20 (td, J = 8.2, 6.3, 1H), 7.48 (s, | | |
| | | | | 1H) | | |
| 24 | 3-[5-(1,1-difluoroethyl)-2,6- | Achiral | Intermediates | (400 MHz, DMSO-d₆) δ: ppm | m/z 552.2 | — |
| | dimethoxypyridin-3-yl]-1 - | | 15 and 20 | 1.93 (t, J = 18.9, 3H), 2.97 (t, | (M + H)+ (ES+), at | |
| | [2,6-difluoro-4- | | Procedure 2 | J = 5.7, 2H), 3.28 (s, 3H), 3.65 (t, | 6.20 min, 100% | |
| | (methoxymethyl)benzyl]-6- | | | J = 5.7, 2H), 3.80 (s, 3H), 3.93 (s, | | |
| | fluoro-1,3,4,5-tetrahydro- | | | 3H), 4.36 (s, 2H), 5.01 (s, 2H), | | |
| | 2H-1,3-benzodiazepin-2-one | | | 6.92-7.04 (m, 3H), 7.26-7.36 (m, | | |
| | | | | 2H), 7.53 (s, 1H) | | |
| 25 | 1-(2,6-difluoro-4- | Achiral | Intermediates | (400 MHz, DMSO-d₆) δ: ppm | m/z 552.1 | — |
| | methoxybenzyl)-3-[2,6- | | 16 and 22 | 2.91 (t J = 6.3, 2H), 3.15 (s, 3H), | (M + H)+ (ES+), at | |
| | dimethoxy-5- | | Procedure 4 | 3.61 (t, J = 6.4, 2H), 3.68 (s, 3H), | 5.02 min, 100% | |
| | (methylsulfonyl)pyridin-3- | | | 3.84 (s, 3H), 3.99 (s, 3H), 4.93 (s, | | |
| | yl]-6-fluoro-1,3,4,5- | | | 2H), 6.61 (d, 2H), 6.97 (ddd, | | |
| | tetrahydro-2H-1,3- | | | J = 9.2, 6.6, 2.7, 1H), 7.21-7.33 (m, | | |
| | benzodiazepin-2-one | | | 2H), 7.72 (s, 1H) | | |
| 26 | 1-(4-bromo-2,6- | Achiral | Intermediates | (500 MHz, DMSO-d₆) δ: ppm | m/z 521.1, 523.1 | — |
| | difluorobenzyl)-6-fluoro-3- | | 2 and 17 | 2.17 (s, 3H) 2.39 (s, 3H), 2.97 (t, | (M + H)+ (ES+), at | |
| | [4-methyl-3- | | Procedure 3 | J = 6.4, 2H), 3.78 (t, J = 6.4, 2H), | 6.51 min, 100% | |
| | (methylsulfanyl)phenyl]- | | | 5.03 (s, 2H), 6.72 (dd, J = 8.1, 2.0, | | |
| | 1,3,4,5-tetrahydro-2H-1,3- | | | 1H), 6.84 (d, J = 1.8, 1H), 6.99- | | |
| | benzodiazepin-2-one | | | 7.08 (m, 2H), 7.28-7.36 (m, 2H), | | |
| | | | | 7.43 (d, J = 7.3, 2H) | | |
| 27 | 1-(4-bromo-2,6- | Achiral | Intermediates | (400 MHz, DMSO-d₆) δ: ppm | m/z 553.0, 555.0 | — |
| | difluorobenzyl)-6-fluoro-3- | | 3 and 17 | 2.53 (s, 3H), 2.95 (t, J = 5.7, 2H), | (M + H)+ (ES+), at | |
| | [4-methyl-3- | | Procedure 3 | 3.14 (s, 3H), 3.75 (t, J = 6.0, 2H), | 5.52 min, 100% | |
| | (methylsulfonyl)phenyl]- | | | 5.07 (s, 2H), 7.04 (t, J = 9.1, 1H), | | |
| | 1,3,4,5-tetrahydro-2H-1,3- | | | 7.23-7.53 (m, 7H) | | |
| | benzodiazepin-2-one | | | | | |
| 28 | 1-(2,6-difluoro-4- | Achiral | Intermediates | (400 MHz, CDCl₃) δ: ppm 0.89 (t, | m/z 517.2 | — |
| | propylbenzyl)-6-fluoro-3-[4- | | 3 and 21 | J = 7.4, 3H), 1.50-1.67 (m, 2H), | (M + H)+ (ES+), at | |
| | methyl-3- | | Procedure 2 | 2.49 (t, J = 7.6, 2H), 2.62 (s, 3H), | 5.93 min, 100% | |
| | (methylsulfonyl)phenyl]- | | | 3.04 (s, 3H), 3.09 (t, J = 6.6, 2H) | | |
| | 1,3,4,5-tetrahydro-2H-1,3- | | | 3.83 (t, J = 6.6, 2H), 5.13 (s, 2H), | | |
| | benzodiazepin-2-one | | | 6.61 (d, J = 8.6, 2H), 6.87 (t, J = 8.4, | | |
| | | | | 1H), 7.12 (d, J = 8.2, 1H), 7.15- | | |
| | | | | 7.30 (m, 3H), 7.72 (s, 1H) | | |
| 29 | 1-(4-bromo-2,6- | Achiral | Intermediates | (400 MHz, DMSO-d₆) δ: ppm | m/z 549.3, 551.3 | — |
| | difluorobenzyl)-3-[2,4- | | 4 and 17 | 1.86 (s, 3H), 2.52 (s, 3H), 2.96 (t, | (M + H)+ (ES+), at | |
| | dimethyl-5- | | Procedure 2 | J = 6.1, 2H), 3.15 (s, 3H), 3.60 (br. | 5.47 min, 100% | |
| | (methylsulfonyl)phenyl]- | | | s., 2H), 5.02 (s, 2H), 7.13 (dd, | | |
| | 1,3,4,5-tetrahydro-2H-1,3- | | | J = 8.3, 6.4, 1H), 7.21-7.26 (m, | | |
| | benzodiazepin-2-one | | | 2H), 7.30 (t, J = 7.7, 1H), 7.38 (d, | | |
| | | | | J = 7.0, 2H), 7.44 (d, J = 8.0, 1H), | | |
| | | | | 7.59 (s, 1H) | | |
| 30 | 1-[2,6-difluoro-4-(propan-2- | Achiral | Intermediates | (400 MHz, CDCl₃) δ: ppm 1.29 | m/z 529.3 | — |
| | yloxy)benzyl]-3-[2,4- | | 4 and 18 | (d, J = 6.0, 6H), 1.99 (s, 3H), 2.60 | (M + H)+ (ES+), at | |
| | dimethyl-5- | | Procedure 2 | (s, 3H), 3.05 (s, 5H), 3.70 (br. s., | 5.50 min, 100% | |
| | (methylsulfonyl)phenyl]- | | | 2H), 4.40 (p, J = 6.1, 1H), 5.07 (br. | | |
| | 1,3,4,5-tetrahydro-2H-1,3- | | | s., 2H), 6.31 (d, J = 9.7, 2H), 7.08- | | |
| | benzodiazepin-2-one | | | 7.16 (m, 3H), 7.24-7.33 (m, 2H), | | |
| | | | | 7.83 (s, 1H) | | |
| 31 | 1-(4-bromo-2,6- | Achiral | Intermediates | (400 MHz, DMSO-d₆) δ: ppm | m/z 567.0, 569.0 | — |
| | difluorobenzyl)-3-[2,4- | | 5 and 17 | 1.87 (s, 3H), 2.55 (s, 3H), 3.01 (t, | (M + H)+ (ES+), at | |
| | dimethyl-5- | | Procedure 2 | J = 5.6, 2H), 3.18 (s, 3H), 3.67 (br. | 5.73 min, 100% | |
| | (methylsulfonyl)phenyl]-6- | | | s, 2H), 5.01 (br. s., 2H), 7.02-7.10 | | |
| | fluoro-1,3,4,5-tetrahydro- | | | (m, 1H), 7.28 (s, 1H), 7.31-7.40 | | |
| | 2H-1,3-benzodiazepin-2-one | | | (m, 2H), 7.43 (d, J = 7.3, 2H), 7.62 | | |
| | | | | (s, 1H) | | |
| 32 | 1-(4-bromo-2,6- | Achiral | Intermediates | (400 MHz, DMSO-d₆) δ: ppm | m/z 540.1, 542.1 | — |
| | difluorobenzyl)-3-[5-(1,1- | | 6 and 17 | 1.97 (t, J = 19.2, 3H), 2.53 (s, 3H), | (M + H)+ (ES+), at | |
| | difluoroethyl)-6- | | Procedure 2 | 2.98 (t, J = 6.3, 2H), 3.82 (t, J = 6.3, | 5.81 min, 100% | |
| | methylpyridin-3-yl]-6- | | | 2H), 5.09 (s, 2H), 7.03-7.12 (m, | | |
| | fluoro-1,3,4,5-tetrahydro- | | | 1H), 7.30-7.40 (m, 2H), 7.43 (d, | | |
| | 2H-1,3-benzodiazepin-2-one | | | J = 7.0, 2H), 7.50 (d, J = 2.1, 1H), | | |
| | | | | 8.27 (d, J = 1.8, 1H) | | |
| 33 | 3-[5-(1,1-difluoroethyl)-6- | Achiral | Intermediates | (400 MHz, DMSO-d₆) δ: ppm | m/z 506.1 | — |
| | methylpyridin-3-yl]-1-[2,6- | | 6 and 20 | 1.97 (t, J = 19.1, 3H), 2.53 (s, 3H), | (M + H)+ (ES+), at | |
| | difluoro-4- | | Procedure 1 | 2.97 (t, J = 6.3, 2H), 3.27 (s, 3H), | 5.42 min, 100% | |
| | (methoxymethyl)benzyl]-6- | | | 3.77-3.86 (m, 2H), 4.36 (s, 2H), | | |
| | fluoro-1,3,4,5-tetrahydro- | | | 5.13 (s, 2H), 6.96 (d, J = 8.2, 2H), | | |
| | 2H-1,3-benzodiazepin-2-one | | | 7.05 (t, J = 8.6, 1H), 7.28-7.41 (m, | | |
| | | | | 2H), 7.49 (d, J = 2.3, 1H), 8.27 (d, | | |
| | | | | J = 2.0, 1 H) | | |
| 34 | 3-[5-(1,1-difluoroethyl)-6- | Achiral | Intermediates | (400 MHz, CDCl₃) δ: ppm 0.89 (t, | m/z 504.2 | — |
| | methylpyridin-3-yl]-1-(2,6- | | 6 and 21 | J = 7.3, 3H), 1.50-1.63 (m, 2H), | (M + H)+ (ES+), at | |
| | difluoro-4-propylbenzyl)-6- | | Procedure 2 | 1.93 (t, J = 18.3, 3H), 2.48 (t, | 6.22 min, 100% | |
| | fluoro-1,3,4,5-tetrahydro- | | | J = 7.6, 2H), 2.60 (s, 3H), 3.11 (t, | | |
| | 2H-1,3-benzodiazepin-2-one | | | J = 6.2, 2H), 3.82 (t, J = 6.4, 2H), | | |
| | | | | 5.14 (s, 2H), 6.61 (d, J = 8.4, 2H), | | |
| | | | | 6.87 (t, J = 8.3, 1H), 7.14 (d, J = | | |
| | | | | 8.3, 1H), 7.16-7.25 (m, 1H), 7.50 | | |
| | | | | (s, 1H), 8.29 (s, 1H) | | |
| 35 | 1-(4-bromo-2,6- | Racemic | Intermediates | (400 MHz, DMSO-d₆) δ: ppm | m/z 539.1, 541.1 | — |
| | difluorobenzyl)-3-[5-(1,1- | | 7 and 17 | 1.99 (t, J = 18.9, 3H), 2.07-2.23 | (M + H)+ (ES+), at | |
| | difluoroethyl)-6- | | Procedure 1 | (m, 1H), 2.28-2.45 (m, 2H), 2.57 | 5.96 min, 100% | |
| | methylpyridin-3-yl]-6- | | | (s, 3H), 2.94-2.99 (m, 1H), 3.62- | | |
| | fluoro-1,3,4,5-tetrahydro- | | | 3.78 (m, 1H), 4.71 (d, J = 14.8, | | |
| | 2H-1-benzazepin-2-one | | | 1H), 5.45 (d, J = 14.7, 1H), 7.11- | | |
| | | | | 7.16 (m, 1H), 7.36-7.45 (m, 4H), | | |
| | | | | 7.70-7.79 (m, 1H), 8.41 (s, 1H) | | |
| 36 | 1-(4-bromo-2,6- | Enantiomer 1 | Intermediates | (400 MHz, DMSO-d₆) δ: ppm | m/z 539.2, 541.2 | Chiral purity |
| | difluorobenzyl)-3-[5-(1,1- | | 7 and 17 | 1.99 (t, J = 19.3, 3H), 2.07-2.25 | (M + H)+ (ES+), at | analysis: 1.75 |
| | difluoroethyl)-6- | | Procedure 1, | (m, 1H), 2.27-2.45 (m, 2H), 2.57 | 6.03 min, 100% | min, 99.8% |
| | methylpyridin-3-yl]-6- | | then chiral | (s, 3H), 2.98 (dd, J = 12.9, 6.6, | | (Method J) |
| | fluoro-1,3,4,5-tetrahydro- | | separation | 1H), 3.71 (dd, J = 11.7, 7.4, 1H), | | |
| | 2H-1-benzazepin-2-one | | | 4.72 (d, J = 14.8, 1H), 5.45 (d, | | |
| | | | | J = 14.8, 1H), 7.09-7.16 (m, 1H), | | |
| | | | | 7.34-7.44 (m, 4H), 7.76 (s, 1H), | | |
| | | | | 8.41 (s, 1H) | | |
| 37 | 1-(4-bromo-2,6- | Achiral | Intermediates | (400 MHz, DMSO-d₆) δ: ppm | m/z 542.1, 544.1 | — |
| | difluorobenzyl)-3-[5- | | 11 and 27 | 2.35 (s, 3H), 2.98 (t, J = 6.0, 2H), | (M + H)+ (ES+), at | |
| | (difluoromethoxy)-6- | | Procedure 1 | 3.78 (t, J = 6.2, 2H), 5.09 (s, 2H), | 5.68 min, 100% | |
| | methylpyridin-3-yl]-6- | | | 7.21 (t, J = 73.9, 1H), 7.04-7.15 | | |
| | fluoro-1,3,4,5-tetrahydro- | | | (m, 1H), 7.35 (d, J = 6.1, 3H), 7.43 | | |
| | 2H-1,3-benzodiazepin-2-one | | | (d, J = 7.0, 2H), 8.08 (d, J = 1.5, 1H) | | |
| 38 | 1-(4-bromo-2,6- | Racemic | Intermediates | (400 MHz, CDCl₃) δ: ppm 2.17- | m/z 553.1, 555.1 | — |
| | difluorobenzyl)-6-fluoro-3- | | 9 and 17 | 2.33 (m, 1H), 2.41-2.65 (m, 2H), | (M + H)+ (ES+), at | |
| | [6-methyl-5- | | Procedure 1 | 2.90 (s, 3H), 3.04-3.26 (m, 4H), | 5.16 min, 100% | |
| | (methylsulfonyl)pyridin-3- | | | 3.69 (dd, J = 11.6, 7.3, 1H), 4.66 | | |
| | yl]-1,3,4,5-tetrahydro-2H-1- | | | (d, J = 14.6, 1H), 5.63 (d, J = 14.6, | | |
| | benzazepin-2-one | | | 1H), 6.94-7.07 (m, 3H), 7.15 (d, | | |
| | | | | J = 7.9, 1H), 7.23-7.37 (m, 1H), | | |
| | | | | 8.23 (d, J = 1.2, 1H), 8.60 (d, | | |
| | | | | J = 1.8, 1H) | | |
| 39 | 1-(4-bromo-2,6- | Enantiomer 1 | Intermediates | (400 MHz, METHANOL-d₄) δ: | m/z 553.0, 555.0 | Chiral purity |
| | difluorobenzyl)-6-fluoro-3- | | 9 and 17 | ppm 2.20-2.37 (m, 1H), 2.45-2.60 | (M + H)+ (ES+), at | analysis: 1.26 |
| | [6-methyl-5- | | Procedure 1, | (m, 2H), 2.86 (s, 3H), 3.08-3.17 | 5.09 min, 100% | min, 100% |
| | (methylsulfonyl)pyridin-3- | | then chiral | (m, 1H), 3.22 (s, 3H), 3.78-3.91 | | (Method N) |
| | yl]-1,3,4,5-tetrahydro-2H-1- | | separation | (m, 1H), 4.77 (d, J = 14.4, 1H), | | |
| | benzazepin-2-one | | | 5.63 (d, J = 14.4, 1H), 7.06 (t, | | |
| | | | | J = 8.4, 1H), 7.17 (d, J = 7.4, 2H), | | |
| | | | | 7.30-7.42 (m, 2H), 8.30 (s, 1H), | | |
| | | | | 8.52 (s, 1H) | | |
| 40 | 3-[5-(1,1-difluoroethyl)-6- | Enantiomer 1 | Intermediates | (400 MHz, DMSO-d₆) δ: ppm | m/z 505.25 | Chiral purity |
| | methylpyridin-3-yl]-1-[2,6- | | 7 and 20 | 1.97 (t, J = 19.2, 3H), 2.06-2.17 | (M + H)+ (ES+), at | analysis: 1.26 |
| | difluoro-4- | | Procedure 1, | (m, 1H), 2.34-2.46 (m, 2H), 2.55 | 5.53 min, 100% | min, 100% |
| | (methoxymethyl)benzyl]-6- | | then chiral | (s, 3H), 2.95 (dd, J = 12.7, 6.4, | | (Method G) |
| | fluoro-1,3,4,5-tetrahydro- | | separation | 1H), 3.25 (s, 3H), 3.68 (dd, | | |
| | 2H-1-benzazepin-2-one | | | J = 12.9, 6.7, 1H), 4.33 (s, 2H), | | |
| | | | | 4.71 (d, J = 14.8, 1H), 5.51 (d, | | |
| | | | | J = 14.8, 1H), 6.92 (d, J = 8.3, 2H), | | |
| | | | | 7.04-7.15 (m, 1H), 7.36 (s, 2H), | | |
| | | | | 7.75 (s, 1H), 8.39 (s, 1H) | | |
| 41 | 3-({3-[5-(1,1-difluoroethyl)- | Enantiomer 1 | Intermediates | (400 MHz, DMSO-d₆) δ: ppm | m/z 468.20 | Chiral purity |
| | 6-methylpyridin-3-yl]-6- | | 7 and 31 | 1.98 (t, J = 19.2, 3H), 2.13-2.24 | (M + H)+ (ES+), at | analysis: 1.26 |
| | fluoro-2-oxo-2,3,4,5- | | Procedure 1, | (m, 18H), 2.37-2.53 (m, 2H), 2.56 | 5.24 min, 97% | min, 100% |
| | tetrahydro-1H-1-benzazepin- | | then chiral | (d, J = 2.2, 3H), 3.00 (dd, J = 13.5, | | (Method H) |
| | 1-yl}methyl)-4- | | separation | 6.4, 1H), 3.88 (dd, J = 12.1, 7.4, | | |
| | fluorobenzonitrile | | | 1H), 4.99 (d, J = 15.7, 1H), 5.16 (d, | | |
| | | | | J = 15.7, 1H), 7.14 (t, J = 8.7, 1H), | | |
| | | | | 7.26-7.44 (m, 3H), 7.82 (ddd, | | |
| | | | | J = 8.3, 4.8, 2.1, 1H), 7.84-7.92 (m, | | |
| | | | | 2H), 8.47 (s, 1H) | | |

Cloning, Baculovirus generation, large-scale infection of SF21 cells and membrane preparation. Human OX₁ or OX₂ receptor were cloned into Invitrogen's (ThermoFisher Scientific, UK) Bac-to-Bac Baculovirus Expression System. P0 baculovirus was generated by transfecting SF9 cells with bacmid DNA using Cellfectin® II transfection reagent (ThermoFisher Scientific, UK, Catalog number 10362-100). Following P0 generation P1 virus was then generated ready for large scale infection and membrane preparation. SF21 cells were grown in expression medium ESF921 (Expression Systems, USA, catalog number 96-001-01) supplemented with 10% heat-inactivated FBS and 1% Pen/Strep and were infected at a cell density of 2.5×10⁶ cells/mL and a multiplicity of infection of 1.0 for both human OX₁R and OX₂R. Incubation was carried out at over 48 h in a shaking incubator set at 27° C. The cell culture was then centrifuged at 2,500 rcf for 10 min at 4° C. The pellets were resuspended in cold PBS supplemented with Roche's Complete EDTA-free protease inhibitor cocktail tablets (Roche Applied Sciences, catalog number 05056489001), 1 mM PMSF and 1 mM EDTA. The resuspended cell paste was then centrifuged at 3,273 rcf for 12 min at 4° C. The supernatant was discarded and the pellet frozen at −80° C. The cell pellet from a 4 L culture was resuspended in buffer containing 50 mM HEPES pH 7.5, 150 mM NaCl, 8 Roche EDTA-Free protease inhibitor cocktail tablets and 1 mM PMSF. The suspension was left stirring at rt for 1 h and then homogenised for 90 s at 9,500 rpm using a VDI 25 (VWR, USA) homogeniser. The cells were then lysed using a Microfluidiser processor M-110L Pneumatic (Microfluidics, USA) at 60 PSI and membranes collected by ultracentrifugation at 204.7 k×g for 1 h. The supernatant was discarded and the pellet was resuspended and homogenised in 90 s at 9,500 rpm in 50 mL (25 mL for each 2 L culture) of buffer containing 50 mM Hepes pH 7.5, 150 mM NaCl, 3 Roche EDTA-free protease inhibitor cocktail tablets and 1 mM PMSF. The resulting membranes were then stored at −80° C.

[³H]-radioligand binding assay. After thawing, membrane homogenates were resuspended in the binding buffer (8.5 mM HEPES, pH 7.4, 1.3 mM CaCl₂, 1.2 mM MgSO₄, 118 mM NaCl, 4.7 mM KCl, 4 mM NaHCO₃, 1.2 mM KH₂PO₄, 11 mM glucose) to a final assay concentration of 6.4 μg (OX₁) or 1.4 μg (OX₂) protein per well. Saturation isotherms were determined by the addition of various concentrations (0-30 nM) of [³H]-4-(2,6-difluoro-4-methoxybenzyl)-2-(5,6-dimethoxypyridin-3-yl)-2H-1,2,4-benzothiadiazin-3(4H)-one 1,1-dioxide (Christopher et al, MedChemComm., 2015, 6, 947-955) in a total reaction volume of 250 μL for 90 min at rt. At the end of the incubation, membranes were filtered onto a 96-well GF/B filter pre-incubated with 0.5% polyethylenimine, with a Tomtec cell harvester and washed 5 times with 0.5 mL distilled water. Non-specific binding (NSB) was measured in the presence of 3.33 μM [4-(5-chloro-1,3-benzoxazol-2-yl)-7-methyl-1,4-diazepan-1-yl][5-methyl-2-(2H-1,2,3-triazol-2-yl)phenyl]methanone (Wertz et al, Angew. Chem. Int. Ed., 2011, 50, 11511-11515). Radioactivity on the filter was counted (1 min) on a Microbeta radiometric plate counter (Perkin Elmer) after addition of 50 μL of scintillation fluid (LabLogic: Part #SG-BXX-14). For competition binding experiments, membranes were incubated with [³H]-4-(2,6-difluoro-4-methoxybenzyl)-2-(5,6-dimethoxypyridin-3-yl)-2H-1,2,4-benzothiadiazin-3(4H)-one 1,1-dioxide at a concentration equal to the K_{D} value of the radioligand (1.5 nM for OX₁ and 0.75 nM for OX₂ receptors respectively) and 10 concentrations of the inhibitory compound (between the ranges of 10 μM-0.94 pmol). IC₅₀ values were derived from the inhibition curve and the equilibrium dissociation constant (Kᵢ) values were calculated using the Cheng-Prusoff equation. The pKᵢ values (where pKᵢ=−log₁₀ Kᵢ) of compounds of the invention are shown in Table 3.

Generation of CHO stable cell lines expressing the human OX₁R and OX₂R. Stable cell lines for the human OX₁R and OX₂R receptors were generated through the transfection of CHO cells using the transfection reagent Genejuice (Novagen number 70967) and cDNA coding for either the human OX₁R or OX₂R. Cells were cultured in Sigma Nutrient Mixture F-12 Ham media (catalog number N6658) supplemented with 10% heat-inactivated FBS. Forty-eight hours after transfection, cells were harvested and placed under geneticin selection by culturing in Sigma Nutrient Mixture F-12 Ham media supplemented with 10% heat-inactivated FBS and geneticin (ThermoFisher Catalog number: 10131035). Selection of the final stable clone for each receptor was made on the basis of receptor expression, assay signal to noise and robustness though increasing passage. Selected clones for the human OX₁R and OX₂R were then frozen down into liquid nitrogen using Cell Freezing Medium-DMSO (Sigma-Aldrich catalog number: C6164).

IPone accumulation assay. A CHO cell line stably expressing either human OX₁ or OX₂ receptor was used with the IPone HTRF assay kit (CisBio: Part #62IPAPEJ) to measure receptor activation. The assay was optimised to measure the ability (potency; fpKb) of antagonists to reduce agonist (orexin A)-induced inositol phosphate turnover. Briefly, cells were plated onto half area 96-well white walled plates at a density of 12,500 cells/well. Sixteen hours post-plating cell growth media was replaced with antagonist concentration-response curve diluted in stimulation buffer (supplied with the kit). Cells were incubated in a humidified incubator (37° C.) for 30 min before an EC₈₀ challenge concentration (˜20 nM (OX₁) and 40 nM (OX₂)) of orexin A (Tocris catalogue number 1455). After 30 min stimulation in a humidified incubator (37° C.) the assay was terminated by the addition of detection mixture as per manufacturer's instructions. The concentration of compound which reduced orexin-A stimulated turnover of inositol phosphates by 50% (IC₅₀) was calculated by fitting to a four parameter sigmoidal dose response curve. The IC₅₀ values were converted to fpK_{B} values using the orexin A pEC₅₀ value estimated on each plate as well as the challenge concentration interpolated from the orexin A control curve. The fpK_{B} values of compounds of the invention are shown in Table 3.

TABLE 3

| | | | pKᵢ average | | fpK_{b} average | |
|---|---|---|---|---|---|---|
| No. | Name | Structure | OX₁ | OX₂ | OX₁ | OX₂ |
| 1 | 1-(4-bromo-2,6- difluorobenzyl)- 3-[3-(1,1- difluoroethyl)-4- methylphenyl]-6- fluoro-1,3,4,5- tetrahydro-2H- 1,3- benzodiazepin-2- one | 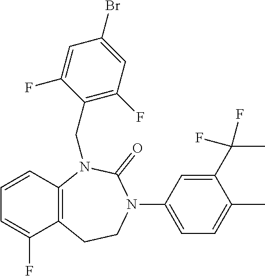 [SMILES: Cc1ccc(N2CCc3c(F)cccc3N(Cc3c(F)cc(Br)cc3F)C2=O)cc1C(C)(F)F] | 8.7 | 6.7 | 6.8 | <5.5 |
| 2 | 1-(4-bromo-2,6- difluorobenzyl)- 3-{5-(1,1- difluoroethyl)-6- [(²H₃)methyloxy] pyridin-3-yl}-6- fluoro-1,3,4,5- tetrahydro-2H- 1,3- benzodiazepin-2- one | 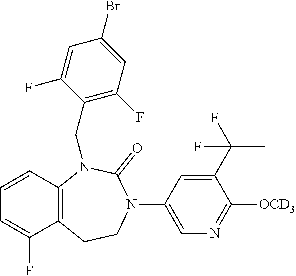 | 9.2 | 6.8 | 7.7 | <5.4 |
| 3 | 1-(4-bromo-2,6- difluorobenzyl)- 3-[5-(1,1- difluoroethyl)-6- methoxypyridin- 3-yl]-6-fluoro- 1,3,4,5- tetrahydro-2H- 1,3- benzodiazepin-2- one | 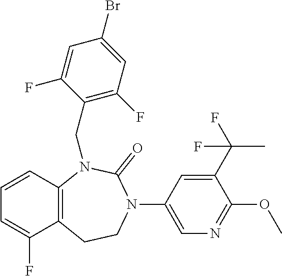 [SMILES: COc1ncc(N2CCc3c(F)cccc3N(Cc3c(F)cc(Br)cc3F)C2=O)cc1C(C)(F)F] | 8.9 | <7.1 | 7.3 | <6.1 |
| 4 | 3-[5-(1,1- difluoroethyl)-6- methoxypyridin- 3-yl]-6-fluoro-1- (2-fluoro-4- methoxybenzyl)- 1,3,4,5- tetrahydro-2H- 1,3- benzodiazepin-2- one | 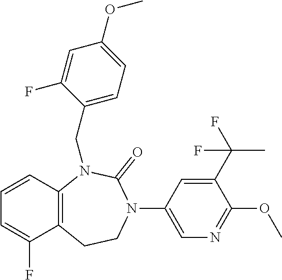 [SMILES: COc1ccc(CN2C(=O)N(c3cnc(OC)c(C(C)(F)F)c3)CCc3c(F)cccc32)c(F)c1] | 9.1 | 7.2 | 7.8 | <5.4 |
| 5 | 3-[5-(1,1- difluoroethyl)-6- methoxypyridin- 3-yl]-1-(2,6- difluoro-4- {[(²H₃)methyloxy] methyl}benzyl)- 6-fluoro-1,3,4,5- tetrahydro-2H- 1,3- benzodiazepin-2- one | 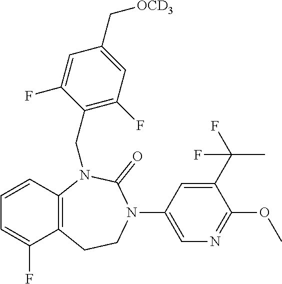 | 9.5 | 6.8 | 8.8 | <5.7 |
| 6 | 3-[5-(1,1- difluoroethyl)-6- methoxypyridin- 3-yl]-1-{2,6- difluoro-4- [(²H₃)methyloxy] benzyl}-6-fluoro- 1,3,4,5- tetrahydro-2H- 1,3- benzodiazepin-2- one | 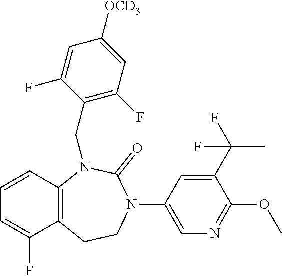 | 9.4 | 7.0 | 8.0 | 6.4 |
| 7 | 3-[5-(1,1- difluoroethyl)-6- methoxypyridin- 3-yl]-1-[2,6- difluoro-4- (methoxymethyl) benzyl]-6-fluoro- 1,3,4,5- tetrahydro-2H- 1,3- benzodiazepin-2- one | 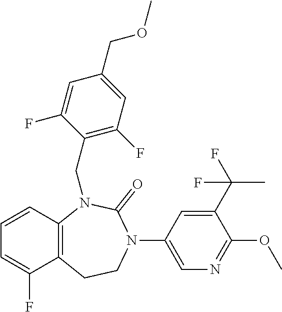 [SMILES: COCc1cc(F)c(CN2C(=O)N(c3cnc(OC)c(C(C)(F)F)c3)CCc3c(F)cccc32)c(F)c1] | 9.0 | 7.1 | 8.1 | <5.4 |
| 8 | 3-[5-(1,1- difluoroethyl)-6- methoxypyridin- 3-yl]-1-(2,6- difluoro-4- methoxybenzyl)- 6-fluoro-1,3,4,5- tetrahydro-2H- 1,3- benzodiazepin-2- one | 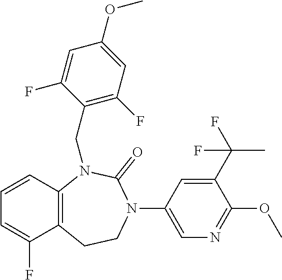 [SMILES: COc1cc(F)c(CN2C(=O)N(c3cnc(OC)c(C(C)(F)F)c3)CCc3c(F)cccc32)c(F)c1] | 9.4 | 6.4 | 8.5 | <6.5 |
| 9 | 1-(4-chloro-2,6- difluorobenzyl)- 3-[5-(1,1- difluoroethyl)-6- methoxypyridin- 3-yl]-6-fluoro- 1,3,4,5- tetrahydro-2H- 1,3- benzodiazepin-2- one | 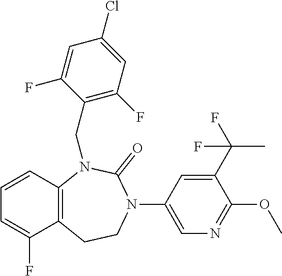 [SMILES: COc1ncc(N2CCc3c(F)cccc3N(Cc3c(F)cc(Cl)cc3F)C2=O)cc1C(C)(F)F] | 9.1 | 6.8 | 7.6 | <5.5 |
| 10 | 1-(4-bromo-2- fluorobenzyl)-3- [5-(1,1- difluoroethyl)-6- methoxypyridin- 3-yl]-6-fluoro- 1,3,4,5- tetrahydro-2H- 1,3- benzodiazepin-2- one | 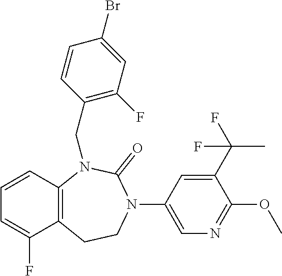 [SMILES: COc1ncc(N2CCc3c(F)cccc3N(Cc3ccc(Br)cc3F)C2=O)cc1C(C)(F)F] | 8.9 | <6.6 | 7.6 | <5.6 |
| 11 | 1-(4-bromo-2,6- difluorobenzyl)- 3-[5-(1,1- difluoroethyl)-6- methoxypyridin- 3-yl]-6-fluoro- 1,3,4,5- tetrahydro-2H-1- benzazepin-2-one (Racemic) | 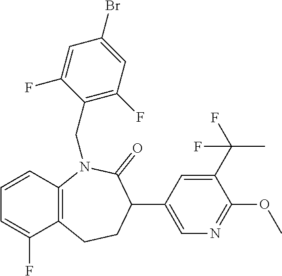 [SMILES: COc1ncc(C2CCc3c(F)cccc3N(Cc3c(F)cc(Br)cc3F)C2=O)cc1C(C)(F)F] | 8.8 | <6.9 | nd | nd |
| 12 | 1-(4-bromo-2,6- difluorobenzyl)- 3-[5-(1,1- difluoroethyl)-6- methoxypyridin- 3-yl]-6-fluoro- 1,3,4,5- tetrahydro-2H-1- benzazepin-2-one (Enantiomer 1) | 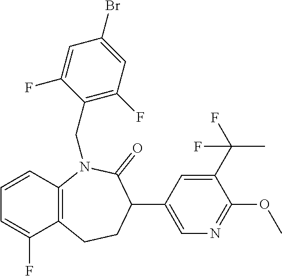 [SMILES: COc1ncc(C2CCc3c(F)cccc3N(Cc3c(F)cc(Br)cc3F)C2=O)cc1C(C)(F)F] | 9.4 | <6.4 | 7.9 | <6.0 |
| 13 | 3-({3-[5-(1,1- difluoroethyl)-6- methoxypyridin- 3-yl]-6-fluoro-2- oxo-2,3,4,5- tetrahydro-1H-1- benzazepin-1- yl}methyl)-4- fluorobenzonitrile (Enantiomer 1) | 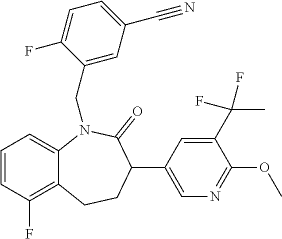 [SMILES: COc1ncc(C2CCc3c(F)cccc3N(Cc3cc(C#N)ccc3F)C2=O)cc1C(C)(F)F] | 9.1 | <6.8 | 7.3 | <6.1 |
| 14 | 1-(4-bromo-2,6- difluorobenzyl)- 6-fluoro-3-[6- methoxy-5- (methylsulfonyl) pyridin-3-yl]- 1,3,4,5- tetrahydro-2H- 1,3- benzodiazepin-2- one | 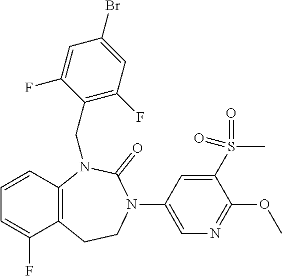 [SMILES: COc1ncc(N2CCc3c(F)cccc3N(Cc3c(F)cc(Br)cc3F)C2=O)cc1S(C)(=O)=O] | 8.5 | <6.6 | 7.1 | <6.1 |
| 15 | 6-fluoro-3-[6- methoxy-5- (methylsulfonyl) pyridin-3-yl]-1- (2,3,6-trifluoro-4- methoxybenzyl)- 1,3,4,5- tetrahydro-2H- 1,3- benzodiazepin-2- one | 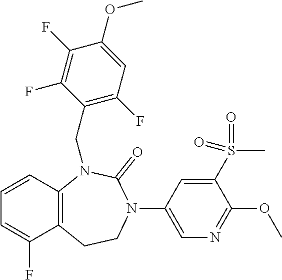 [SMILES: COc1cc(F)c(CN2C(=O)N(c3cnc(OC)c(S(C)(=O)=O)c3)CCc3c(F)cccc32)c(F)c1F] | 8.9 | <6.6 | 6.9 | <5.5 |
| 16 | 1-(2,6-difluoro-4- methoxybenzyl)- 6-fluoro-3-[6- methoxy-5- (methylsulfonyl) pyridin-3-yl]- 1,3,4,5- tetrahydro-2H- 1,3- benzodiazepin-2- one | 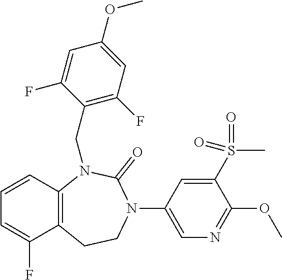 [SMILES: COc1cc(F)c(CN2C(=O)N(c3cnc(OC)c(S(C)(=O)=O)c3)CCc3c(F)cccc32)c(F)c1] | 9.1 | 6.9 | 7.8 | <5.4 |
| 17 | 1-(4-bromo-2- fluoro-6- methylbenzyl)-6- fluoro-3-[6- methoxy-5- (methylsulfonyl) pyridin-3-yl]- 1,3,4,5- tetrahydro-2H- 1,3- benzodiazepin-2- one | 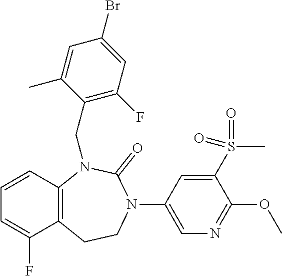 [SMILES: COc1ncc(N2CCc3c(F)cccc3N(Cc3c(C)cc(Br)cc3F)C2=O)cc1S(C)(=O)=O] | 9.1 | 6.8 | 8.0 | <5.6 |
| 18 | 1-(2,4-difluoro-6- methylbenzyl)-6- fluoro-3-[6- methoxy-5- (methylsulfonyl) pyridin-3-yl]- 1,3,4,5- tetrahydro-2H- 1,3- benzodiazepin-2- one | 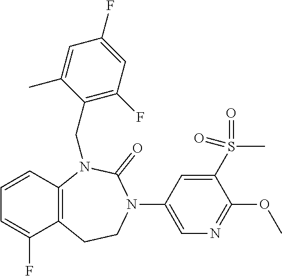 [SMILES: COc1ncc(N2CCc3c(F)cccc3N(Cc3c(C)cc(F)cc3F)C2=O)cc1S(C)(=O)=O] | 8.9 | 6.6 | 8.0 | <5.5 |
| 19 | 1-(4-bromo-2,6- difluorobenzyl)- 6-fluoro-3-[6- methoxy-5- (methylsulfonyl) pyridin-3-yl]- 1,3,4,5- tetrahydro-2H-1- benzazepin-2-one (Racemic) | 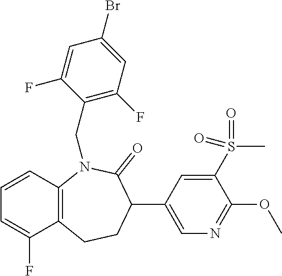 [SMILES: COc1ncc(C2CCc3c(F)cccc3N(Cc3c(F)cc(Br)cc3F)C2=O)cc1S(C)(=O)=O] | 8.4 | <6.6 | 7.5 | <5.5 |
| 20 | 1-(4-bromo-2,6- difluorobenzyl)- 6-fluoro-3-[6- methoxy-5- (methylsulfonyl) pyridin-3-yl]- 1,3,4,5- tetrahydro-2H-1- benzazepin-2-one (Enantiomer 1) | 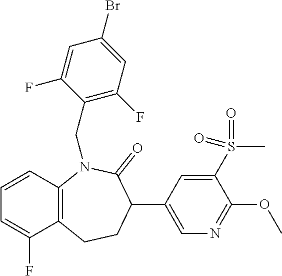 [SMILES: COc1ncc(C2CCc3c(F)cccc3N(Cc3c(F)cc(Br)cc3F)C2=O)cc1S(C)(=O)=O] | 6.7 | <6.2 | nd | nd |
| 21 | 1-(4-bromo-2,6- difluorobenzyl)- 6-fluoro-3-[6- methoxy-5- (methylsulfonyl) pyridin-3-yl]- 1,3,4,5- tetrahydro-2H-1- benzazepin-2-one (Enantiomer 2) | 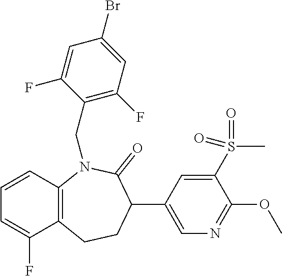 [SMILES: COc1ncc(C2CCc3c(F)cccc3N(Cc3c(F)cc(Br)cc3F)C2=O)cc1S(C)(=O)=O] | 9.5 | 7.1 | 8.0 | <5.8 |
| 22 | 1-(4-chloro-2,6- difluorobenzyl)- 6-fluoro-3-[6- methoxy-5- (methylsulfonyl) pyridin-3-yl]- 1,3,4,5- tetrahydro-2H-1- benzazepin-2-one (Enantiomer 1) | 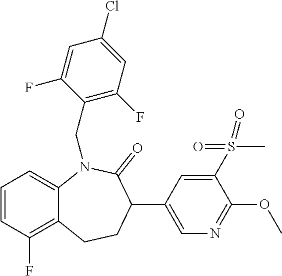 [SMILES: COc1ncc(C2CCc3c(F)cccc3N(Cc3c(F)cc(Cl)cc3F)C2=O)cc1S(C)(=O)=O] | 9.3 | <6.2 | 8.0 | <6.0 |
| 23 | 1-(4-bromo-2,6- difluorobenzyl)- 3-[5-(1,1- difluoroethyl)- 2,6- dimethoxypyridin- 3-yl]-6-fluoro- 1,3,4,5- tetrahydro-2H- 1,3- benzodiazepin-2- one | 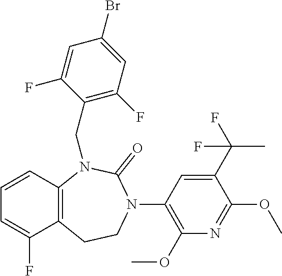 [SMILES: COc1nc(OC)c(C(C)(F)F)cc1N1CCc2c(F)cccc2N(Cc2c(F)cc(Br)cc2F)C1=O] | 8.9 | <6.9 | 7.3 | <5.8 |
| 24 | 3-[5-(1,1- difluoroethyl)- 2,6- dimethoxypyridin- 3-yl]-1-[2,6- difluoro-4- (methoxymethyl) benzyl]-6-fluoro- 1,3,4,5- tetrahydro-2H- 1,3- benzodiazepin-2- one | 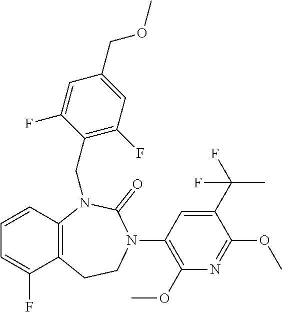 [SMILES: COCc1cc(F)c(CN2C(=O)N(c3cc(C(C)(F)F)c(OC)nc3OC)CCc3c(F)cccc32)c(F)c1] | 9.0 | <6.8 | 7.2 | <5.5 |
| 25 | 1-(2,6-difluoro-4- methoxybenzyl)- 3-[2,6-dimethoxy- 5- (methylsulfonyl) pyridin-3-yl]-6- fluoro-1,3,4,5- tetrahydro-2H- 1,3- benzodiazepin-2- one | 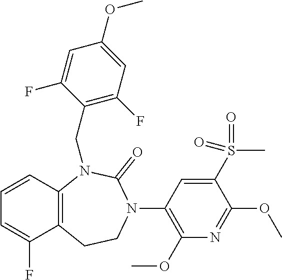 [SMILES: COc1cc(F)c(CN2C(=O)N(c3cc(S(C)(=O)=O)c(OC)nc3OC)CCc3c(F)cccc32)c(F)c1] | 8.8 | <6.9 | 8.0 | <5.4 |
| 26 | 1-(4-bromo-2,6- difluorobenzyl)- 6-fluoro-3-[4- methyl-3- (methylsulfanyl) phenyl]-1,3,4,5- tetrahydro-2H- 1,3- benzodiazepin-2- one | 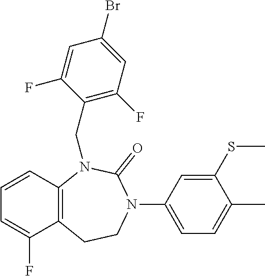 [SMILES: CSc1cc(N2CCc3c(F)cccc3N(Cc3c(F)cc(Br)cc3F)C2=O)ccc1C] | 8.9 | 7.0 | 7.3 | <6.1 |
| 27 | 1-(4-bromo-2,6- difluorobenzyl)- 6-fluoro-3-[4- methyl-3- (methylsulfonyl) phenyl]-1,3,4,5- tetrahydro-2H- 1,3- benzodiazepin-2- one | 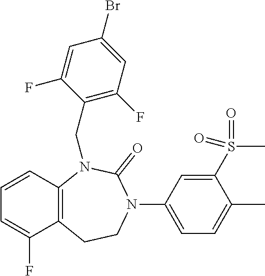 [SMILES: Cc1ccc(N2CCc3c(F)cccc3N(Cc3c(F)cc(Br)cc3F)C2=O)cc1S(C)(=O)=O] | 8.8 | <6.5 | 6.9 | <5.5 |
| 28 | 1-(2,6-difluoro-4- propylbenzyl)-6- fluoro-3-[4- methyl-3- (methylsulfonyl) phenyl]-1,3,4,5- tetrahydro-2H- 1,3- benzodiazepin-2- one | 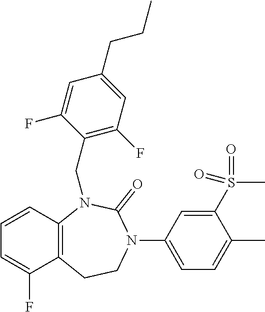 [SMILES: CCCc1cc(F)c(CN2C(=O)N(c3ccc(C)c(S(C)(=O)=O)c3)CCc3c(F)cccc32)c(F)c1] | 8.7 | <6.5 | 7.1 | <5.4 |
| 29 | 1-(4-bromo-2,6- difluorobenzyl)- 3-[2,4-dimethyl- 5- (methylsulfonyl) phenyl]-1,3,4,5- tetrahydro-2H- 1,3- benzodiazepin-2- one | 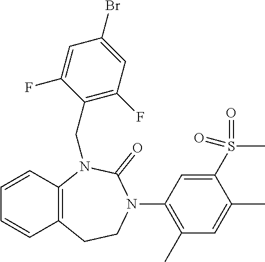 [SMILES: Cc1cc(C)c(S(C)(=O)=O)cc1N1CCc2ccccc2N(Cc2c(F)cc(Br)cc2F)C1=O] | 8.9 | 7.0 | 7.4 | 6.4 |
| 30 | 1-[2,6-difluoro-4- (propan-2- yloxy)benzyl]-3- [2,4-dimethyl-5- (methylsulfonyl) phenyl]-1,3,4,5- tetrahydro-2H- 1,3- benzodiazepin-2- one | 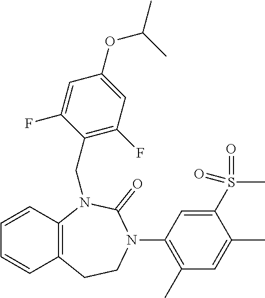 [SMILES: Cc1cc(C)c(S(C)(=O)=O)cc1N1CCc2ccccc2N(Cc2c(F)cc(OC(C)C)cc2F)C1=O] | 8.9 | 6.3 | 7.4 | 6.1 |
| 31 | 1-(4-bromo-2,6- difluorobenzyl)- 3-[2,4-dimethyl- 5- (methylsulfonyl) phenyl]-6-fluoro- 1,3,4,5- tetrahydro-2H- 1,3- benzodiazepin-2- one | 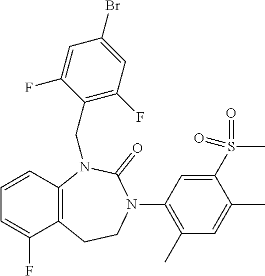 [SMILES: Cc1cc(C)c(S(C)(=O)=O)cc1N1CCc2c(F)cccc2N(Cc2c(F)cc(Br)cc2F)C1=O] | 8.9 | 6.7 | 7.7 | <6.5 |
| 32 | 1-(4-bromo-2,6- difluorobenzyl)- 3-[5-(1,1 difluoroethyl)-6- methylpyridin-3- yl]-6-fluoro- 1,3,4,5- tetrahydro-2H- 1,3- benzodiazepin-2- one | 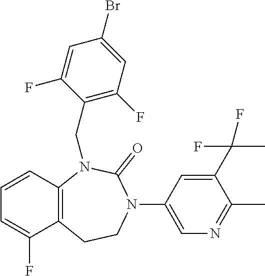 [SMILES: Cc1ncc(N2CCc3c(F)cccc3N(Cc3c(F)cc(Br)cc3F)C2=O)cc1C(C)(F)F] | 9.2 | 7.3 | 7.2 | <6.2 |
| 33 | 3-[5-(1,1- difluoroethyl)-6- methylpyridin-3- yl]-1-[2,6- difluoro-4- (methoxymethyl) benzyl]-6-fluoro- 1,3,4,5- tetrahydro-2H- 1,3- benzodiazepin-2- one | 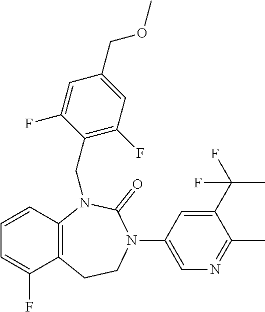 [SMILES: COCc1cc(F)c(CN2C(=O)N(c3cnc(C)c(C(C)(F)F)c3)CCc3c(F)cccc32)c(F)c1] | 9.2 | <6.6 | 7.8 | 6.0 |
| 34 | 3-[5-(1,1- difluoroethyl)-6- methylpyridin-3- yl]-1-(2,6- difluoro-4- propylbenzyl)-6- fluoro-1,3,4,5- tetrahydro-2H- 1,3- benzodiazepin-2- one | 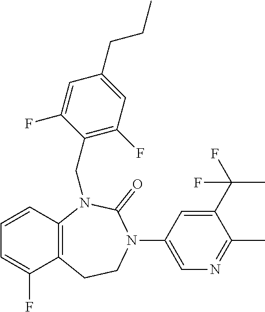 [SMILES: CCCc1cc(F)c(CN2C(=O)N(c3cnc(C)c(C(C)(F)F)c3)CCc3c(F)cccc32)c(F)c1] | 8.8 | <6.6 | 7.1 | <5.6 |
| 35 | 1-(4-bromo-2,6- difluorobenzyl)- 3-[5-(1,1- difluoroethyl)-6- methylpyridin-3- yl]-6-fluoro- 1,3,4,5- tetrahydro-2H-1- benzazepin-2-one (Racemic) | 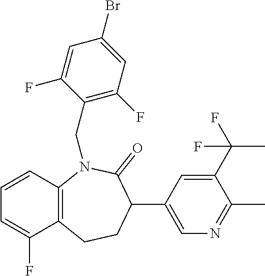 [SMILES: Cc1ncc(C2CCc3c(F)cccc3N(Cc3c(F)cc(Br)cc3F)C2=O)cc1C(C)(F)F] | 8.7 | <6.9 | 7.2 | <5.6 |
| 36 | 1-(4-bromo-2,6- difluorobenzyl)- 3-[5-(1,1- difluoroethyl)-6- methylpyridin-3- yl]-6-fluoro- 1,3,4,5- tetrahydro-2H-1- benzazepin-2-one (Enantiomer 1) | 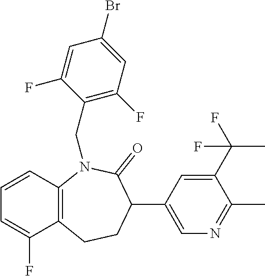 [SMILES: Cc1ncc(C2CCc3c(F)cccc3N(Cc3c(F)cc(Br)cc3F)C2=O)cc1C(C)(F)F] | 9.2 | 6.7 | 7.7 | <5.7 |
| 37 | 1-(4-bromo-2,6- difluorobenzyl)- 3-[5- (difluoromethoxy)- 6- methylpyridin-3- yl]-6-fluoro- 1,3,4,5- tetrahydro-2H- 1,3- benzodiazepin-2- one | 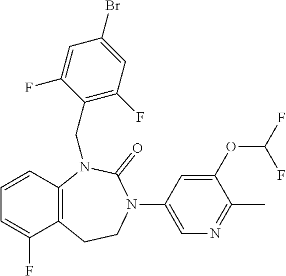 [SMILES: Cc1ncc(N2CCc3c(F)cccc3N(Cc3c(F)cc(Br)cc3F)C2=O)cc1OC(F)F] | 8.8 | 6.9 | 7.7 | <5.4 |
| 38 | 1-(4-bromo-2,6- difluorobenzyl)- 6-fluoro-3-[6- methyl-5- (methylsulfonyl) pyridin-3-yl]- 1,3,4,5- tetrahydro-2H-1- benzazepin-2-one (Racemic) | 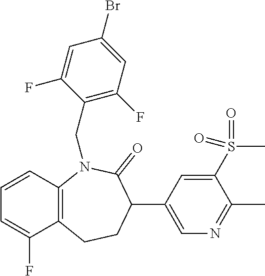 [SMILES: Cc1ncc(C2CCc3c(F)cccc3N(Cc3c(F)cc(Br)cc3F)C2=O)cc1S(C)(=O)=O] | 8. | <6.5 | 7.7 | <6.4 |
| 39 | 1-(4-bromo-2,6- difluorobenzyl)- 6-fluoro-3-[6- methyl-5- (methylsulfonyl) pyridin-3-yl]- 1,3,4,5- tetrahydro-2H-1- benzazepin-2-one (Enantiomer 1) | 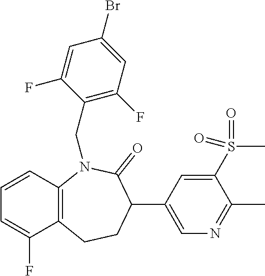 [SMILES: Cc1ncc(C2CCc3c(F)cccc3N(Cc3c(F)cc(Br)cc3F)C2=O)cc1S(C)(=O)=O] | 8.9 | <6.3 | 7.5 | <5.6 |
| 40 | 3-[5-(1,1- difluoroethyl)-6- methylpyridin-3- yl]-1-[2,6- difluoro-4- (methoxymethyl) benzyl]-6-fluoro- 1,3,4,5- tetrahydro-2H-1- benzazepin-2-one (Enantiomer 1) | 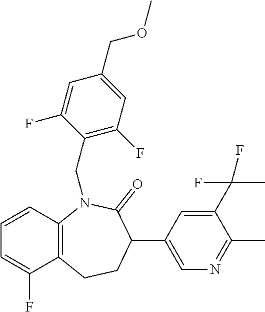 [SMILES: COCc1cc(F)c(CN2C(=O)C(c3cnc(C)c(C(C)(F)F)c3)CCc3c(F)cccc32)c(F)c1] | 8.8 | <7.8 | nd | nd |
| 41 | 3-({3-[5-(1,1- difluoroethyl)-6- methylpyridin-3- yl]-6-fluoro-2- oxo-2,3,4,5- tetrahydro-1H-1- benzazepin-1- yl}methyl)-4- fluorobenzonitrile (Enantiomer 1) | 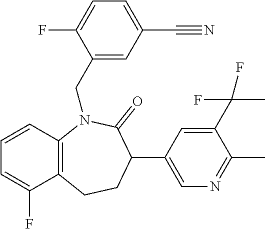 [SMILES: Cc1ncc(C2CCc3c(F)cccc3N(Cc3cc(C#N)ccc3F)C2=O)cc1C(C)(F)F] | 8.9 | <6.3 | nd | nd |

nd = not determined.

1. A compound of formula (1) 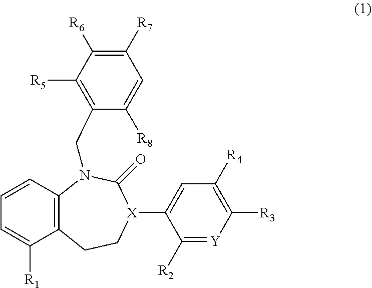
and salts thereof, wherein
X is CH or N;
Y is CH or N;
R₁ is H or F;
R₂ is H, C₁-C₃ alkyl or C₁-C₃ alkoxy;
R₃ is C₁-C₃ alkyl or C₁-C₃ alkoxy;
R₄ is SO(n)CH₃ where n is 0-2 or C₁-C₃ alkyl or C₁-C₃ alkoxy where the alkyl or alkoxy groups are optionally substituted with one or more fluorine atoms;
R₅ is H, halo, cyano, C₁-C₃ alkyl or C₁-C₃ alkoxy;
R₆ is H, halo, cyano, C₁-C₃ alkyl or C₁-C₃ alkoxy;
R₇ is H, halo, cyano, C₁-C₃ alkyl or C₁-C₃ alkoxy; wherein the C₁-C₃ alkyl group can be substituted with C₁-C₃ alkoxy; and
R₈ is H or F.

2. The compound according to claim 1 wherein X is CH.

3. The compound according to claim 1 wherein X is N.

4. The compound according to any one of claims 1-3 wherein Y is CH.

5. The compound according to any one of claims 1-3 wherein Y is N.

6. The compound according to any one of claims 1-5 wherein R₁ is H.

7. The compound according to any one of claims 1-5 wherein R₁ is F.

8. The compound according to any one of claims 1-7 wherein R₂ is H, CH₃ or OCH₃.

9. The compound according to any one of claims 1-8 wherein R₃ is CH₃, OCH₃, CD₃ or OCD₃.

10. The compound according to any one of claims 1-9 wherein R₄ is SCH₃, SO₂CH₃, CF₂CH₃ or OCF₂H.

11. The compound according to any one of claims 1-10 wherein R₅ is H, F or CH₃.

12. The compound according to any one of claims 1-11 wherein R₆ is H, CN or F.

13. The compound according to any one of claims 1-12 wherein R₇ is H, F, Cl, Br, OCH₃, OCH(CH₃)₂, OCD₃, CH₂OCH₃, CH₂OCD₃, CH₂CH₂CH₃.

14. The compound according to any one of claims 1-13 wherein R₈ is H or F.

15. The compound according to any preceding claim wherein the compound is selected from;
1-(4-bromo-2,6-difluorobenzyl)-3-[3-(1,1-difluoroethyl)-4-methylphenyl]-6-fluoro-1,3,4,5-tetrahydro-2H-1,3-benzodiazepin-2-one
1-(4-bromo-2,6-difluorobenzyl)-3-{5-(1,1-difluoroethyl)-6-[(2H₃)methyloxy]pyridin-3-yl}-6-fluoro-1,3,4,5-tetrahydro-2H-1,3-benzodiazepin-2-one
1-(4-bromo-2,6-difluorobenzyl)-3-[5-(1,1-difluoroethyl)-6-methoxypyridin-3-yl]-6-fluoro-1,3,4,5-tetrahydro-2H-1,3-benzodiazepin-2-one
3-[5-(1,1-difluoroethyl)-6-methoxypyridin-3-yl]-6-fluoro-1-(2-fluoro-4-methoxybenzyl)-1,3,4,5-tetrahydro-2H-1,3-benzodiazepin-2-one
3-[5-(1,1-difluoroethyl)-6-methoxypyridin-3-yl]-1-(2,6-difluoro-4-{[(²H₃)methyloxy]methyl}benzyl)-6-fluoro-1,3,4,5-tetrahydro-2H-1,3-benzodiazepin-2-one
3-[5-(1,1-difluoroethyl)-6-methoxypyridin-3-yl]-1-{2,6-difluoro-4-[(²H₃)methyloxy]benzyl}-6-fluoro-1,3,4,5-tetrahydro-2H-1,3-benzodiazepin-2-one
3-[5-(1,1-difluoroethyl)-6-methoxypyridin-3-yl]-1-[2,6-difluoro-4-(methoxymethyl)benzyl]-6-fluoro-1,3,4,5-tetrahydro-2H-1,3-benzodiazepin-2-one
3-[5-(1,1-difluoroethyl)-6-methoxypyridin-3-yl]-1-(2,6-difluoro-4-methoxybenzyl)-6-fluoro-1,3,4,5-tetrahydro-2H-1,3-benzodiazepin-2-one
1-(4-chloro-2,6-difluorobenzyl)-3-[5-(1,1-difluoroethyl)-6-methoxypyridin-3-yl]-6-fluoro-1,3,4,5-tetrahydro-2H-1,3-benzodiazepin-2-one
1-(4-bromo-2-fluorobenzyl)-3-[5-(1,1-difluoroethyl)-6-methoxypyridin-3-yl]-6-fluoro-1,3,4,5-tetrahydro-2H-1,3-benzodiazepin-2-one
1-(4-bromo-2,6-difluorobenzyl)-3-[5-(1,1-difluoroethyl)-6-methoxypyridin-3-yl]-6-fluoro-1,3,4,5-tetrahydro-2H-1-benzazepin-2-one
(3R)-1-(4-bromo-2,6-difluorobenzyl)-3-[5-(1,1-difluoroethyl)-6-methoxypyridin-3-yl]-6-fluoro-1,3,4,5-tetrahydro-2H-1-benzazepin-2-one
3-({3-[5-(1,1-difluoroethyl)-6-methoxypyridin-3-yl]-6-fluoro-2-oxo-2,3,4,5-tetrahydro-1H-1-benzazepin-1-yl}methyl)-4-fluorobenzonitrile
3-({(3R)-3-[5-(1,1-difluoroethyl)-6-methoxypyridin-3-yl]-6-fluoro-2-oxo-2,3,4,5-tetrahydro-1H-1-benzazepin-1-yl}methyl)-4-fluorobenzonitrile
1-(4-bromo-2,6-difluorobenzyl)-6-fluoro-3-[6-methoxy-5-(methylsulfonyl)pyridin-3-yl]-1,3,4,5-tetrahydro-2H-1,3-benzodiazepin-2-one
6-fluoro-3-[6-methoxy-5-(methylsulfonyl)pyridin-3-yl]-1-(2,3,6-trifluoro-4-methoxybenzyl)-1,3,4,5-tetrahydro-2H-1,3-benzodiazepin-2-one
1-(2,6-difluoro-4-methoxybenzyl)-6-fluoro-3-[6-methoxy-5-(methylsulfonyl)pyridin-3-yl]-1,3,4,5-tetrahydro-2H-1,3-benzodiazepin-2-one
1-(4-bromo-2-fluoro-6-methylbenzyl)-6-fluoro-3-[6-methoxy-5-(methylsulfonyl)pyridin-3-yl]-1,3,4,5-tetrahydro-2H-1,3-benzodiazepin-2-one
1-(2,4-difluoro-6-methylbenzyl)-6-fluoro-3-[6-methoxy-5-(methylsulfonyl)pyridin-3-yl]-1,3,4,5-tetrahydro-2H-1,3-benzodiazepin-2-one
1-(4-bromo-2,6-difluorobenzyl)-6-fluoro-3-[6-methoxy-5-(methylsulfonyl)pyridin-3-yl]-1,3,4,5-tetrahydro-2H-1-benzazepin-2-one
(3R)-1-(4-bromo-2,6-difluorobenzyl)-6-fluoro-3-[6-methoxy-5-(methylsulfonyl)pyridin-3-yl]-1,3,4,5-tetrahydro-2H-1-benzazepin-2-one
(3S)-1-(4-bromo-2,6-difluorobenzyl)-6-fluoro-3-[6-methoxy-5-(methylsulfonyl)pyridin-3-yl]-1,3,4,5-tetrahydro-2H-1-benzazepin-2-one
1-(4-chloro-2,6-difluorobenzyl)-6-fluoro-3-[6-methoxy-5-(methylsulfonyl)pyridin-3-yl]-1,3,4,5-tetrahydro-2H-1-benzazepin-2-one
(3R)-1-(4-chloro-2,6-difluorobenzyl)-6-fluoro-3-[6-methoxy-5-(methylsulfonyl)pyridin-3-yl]-1,3,4,5-tetrahydro-2H-1-benzazepin-2-one
1-(4-bromo-2,6-difluorobenzyl)-3-[5-(1,1-difluoroethyl)-2,6-dimethoxypyridin-3-yl]-6-fluoro-1,3,4,5-tetrahydro-2H-1,3-benzodiazepin-2-one
3-[5-(1,1-difluoroethyl)-2,6-dimethoxypyridin-3-yl]-1-[2,6-difluoro-4-(methoxymethyl)benzyl]-6-fluoro-1,3,4,5-tetrahydro-2H-1,3-benzodiazepin-2-one
1-(2,6-difluoro-4-methoxybenzyl)-3-[2,6-dimethoxy-5-(methylsulfonyl)pyridin-3-yl]-6-fluoro-1,3,4,5-tetrahydro-2H-1,3-benzodiazepin-2-one
1-(4-bromo-2,6-difluorobenzyl)-6-fluoro-3-[4-methyl-3-(methylsulfanyl)phenyl]-1,3,4,5-tetrahydro-2H-1,3-benzodiazepin-2-one
1-(4-bromo-2,6-difluorobenzyl)-6-fluoro-3-[4-methyl-3-(methylsulfonyl)phenyl]-1,3,4,5-tetrahydro-2H-1,3-benzodiazepin-2-one
1-(2,6-difluoro-4-propylbenzyl)-6-fluoro-3-[4-methyl-3-(methylsulfonyl)phenyl]-1,3,4,5-tetrahydro-2H-1,3-benzodiazepin-2-one
1-(4-bromo-2,6-difluorobenzyl)-3-[2,4-dimethyl-5-(methylsulfonyl)phenyl]-1,3,4,5-tetrahydro-2H-1,3-benzodiazepin-2-one
1-[2,6-difluoro-4-(propan-2-yloxy)benzyl]-3-[2,4-dimethyl-5-(methylsulfonyl)phenyl]-1,3,4,5-tetrahydro-2H-1,3-benzodiazepin-2-one
1-(4-bromo-2,6-difluorobenzyl)-3-[2,4-dimethyl-5-(methylsulfonyl)phenyl]-6-fluoro-1,3,4,5-tetrahydro-2H-1,3-benzodiazepin-2-one
1-(4-bromo-2,6-difluorobenzyl)-3-[5-(1,1-difluoroethyl)-6-methylpyridin-3-yl]-6-fluoro-1,3,4,5-tetrahydro-2H-1,3-benzodiazepin-2-one
3-[5-(1,1-difluoroethyl)-6-methylpyridin-3-yl]-1-[2,6-difluoro-4-(methoxymethyl)benzyl]-6-fluoro-1,3,4,5-tetrahydro-2H-1,3-benzodiazepin-2-one
3-[5-(1,1-difluoroethyl)-6-methylpyridin-3-yl]-1-(2,6-difluoro-4-propylbenzyl)-6-fluoro-1,3,4,5-tetrahydro-2H-1,3-benzodiazepin-2-one
1-(4-bromo-2,6-difluorobenzyl)-3-[5-(1,1-difluoroethyl)-6-methylpyridin-3-yl]-6-fluoro-1,3,4,5-tetrahydro-2H-1-benzazepin-2-one
(3R)-1-(4-bromo-2,6-difluorobenzyl)-3-[5-(1,1-difluoroethyl)-6-methylpyridin-3-yl]-6-fluoro-1,3,4,5-tetrahydro-2H-1-benzazepin-2-one
1-(4-bromo-2,6-difluorobenzyl)-3-[5-(difluoromethoxy)-6-methylpyridin-3-yl]-6-fluoro-1,3,4,5-tetrahydro-2H-1,3-benzodiazepin-2-one
1-(4-bromo-2,6-difluorobenzyl)-6-fluoro-3-[6-methyl-5-(methylsulfonyl)pyridin-3-yl]-1,3,4,5-tetrahydro-2H-1-benzazepin-2-one
(3R)-1-(4-bromo-2,6-difluorobenzyl)-6-fluoro-3-[6-methyl-5-(methylsulfonyl)pyridin-3-yl]-1,3,4,5-tetrahydro-2H-1-benzazepin-2-one
3-[5-(1,1-difluoroethyl)-6-methylpyridin-3-yl]-1-[2,6-difluoro-4-(methoxymethyl)benzyl]-6-fluoro-1,3,4,5-tetrahydro-2H-1-benzazepin-2-one
(3R)-3-[5-(1,1-difluoroethyl)-6-methylpyridin-3-yl]-1-[2,6-difluoro-4-(methoxymethyl)benzyl]-6-fluoro-1,3,4,5-tetrahydro-2H-1-benzazepin-2-one
3-({3-[5-(1,1-difluoroethyl)-6-methylpyridin-3-yl]-6-fluoro-2-oxo-2,3,4,5-tetrahydro-1H-1-benzazepin-1-yl}methyl)-4-fluorobenzonitrile
3-({(3R)-3-[5-(1,1-difluoroethyl)-6-methylpyridin-3-yl]-6-fluoro-2-oxo-2,3,4,5-tetrahydro-1H-1-benzazepin-1-yl}methyl)-4-fluorobenzonitrile
and salts thereof.

16. The compounds of any one of claims 1 to 15 wherein the salt is a pharmaceutically acceptable salt.

17. The compounds of any one of claims 1 to 16 for use as antagonists of orexin receptor OX₁.

18. The compounds of any one of claims 1 to 16 for use in treating, preventing, ameliorating, controlling or reducing the risk of neurological or psychiatric disorders.

19. The compounds of any one of claims 1 to 16 for use in the treatment or prevention of substance related and addictive disorders (including opioid use disorder, opioid intoxication, opioid withdrawal, other opioid induced disorder, unspecified opioid related disorder, stimulant use disorder, stimulant intoxication, stimulant withdrawal, other stimulant induced disorders, unspecified stimulant related disorder, caffeine related disorders, caffeine intoxication, caffeine withdrawal, unspecified caffeine related disorders, tobacco related disorders, tobacco use disorder, tobacco withdrawal, other tobacco induced disorders, unspecified tobacco related disorder, alcohol use disorder, alcohol intoxication, alcohol withdrawal, unspecified alcohol related disorder, cannabis related disorders, cannabis use disorder, cannabis intoxication, cannabis withdrawal, unspecified cannabis related disorders, hallucinogen related disorders, phencyclidine use disorder, phencyclidine intoxication, other hallucinogen use disorder, hallucinogen persisting perception disorder, unspecified hallucinogen related disorder, inhalant related disorders, inhalant use disorder, inhalant intoxication, other inhalant induced disorders, unspecified inhalant related disorder, sedative, hypnotic or anxiolytic related disorders (including use disorder, intoxication and withdrawal), gambling disorder, internet gaming disorder, addiction to sex or internet use), anxiety disorders (including separation anxiety disorder, specific phobia, social anxiety disorder (social phobia), panic disorder, agoraphobia, generalized anxiety disorder, substance/medication induced anxiety disorder, anxiety disorder due to another medical condition), disruptive mood dysregulation disorder, major depressive disorder (including when specified with anxious distress, mixed features, atypical features, peripartum onset or seasonal pattern), persistent depressive disorder (dysthymia) (including when specified with anxious distress, mixed features, atypical features, peripartum onset or seasonal pattern), premenstrual dysphoric disorder, substance/medication-induced depressive disorder, other specified depressive disorder, unspecified depressive disorder bipolar and related disorders (including bipolar I disorder and bipolar II disorder, particularly, but not exclusively, when these are specified with anxious distress, cyclothymic disorder, substance/medication-induced bipolar and related disorder or bipolar and related disorder due to another medical condition), schizophrenia spectrum and other disorders (including schizotypal personality, delusional disorder, schizophreniform disorder, schizophrenia, schizoaffective disorder, and substance/medication-induced psychotic disorder), conditions associated with trauma and stress (including post traumatic stress disorder, acute stress disorder, adjustment disorders (including when specified with anxiety or with mixed anxiety and depressed mood), obsessive compulsive and related disorders (including obsessive compulsive disorder, body dysmorphia, trichlotillomania, excoriation and obsessive-compulsive and related disorders due to another medical condition), feeding and eating disorders (including binge eating disorder, anorexia nervosa, bulimia nervosa, cachexia, obesity, Prader Willi syndrome)), sleep-wake disorders, neurodegenerative disorders (including dementia), behavioural symptoms of neurodegenerative and other disorders, movement disorders, diabetes, impaired glucose tolerance, cardiovascular disease, diseases related to modulation of sympathetic outflow including hypertension, hypothalamic/pituitary disorders, neuropathic pain, restless leg syndrome, migraine, cluster headache, tension-type headache, trigeminal autonomic cephalalgias, hemicrania continua, trigeminal neuralgia, other headache disorders, hyperalgesia, pain, hyperalgesia, causalgia, and allodynia, acute pain, burn pain, atypical facial pain, back pain, complex regional pain syndrome I and II, arthritic pain, sports injury pain, pain related to infection, irritable bowel syndrome, angina pain, inflammatory disorders, renal/urinary disorders, respiratory disorders, cancer (including prostate cancer, liver cancer, colon cancer, endometrial cancer, pancreatic cancer and cancers associated with other organs of the body including the central nervous system and peripheral nervous system).

20. The compounds of any one of claims 1 to 16 for use in the treatment or prevention of substance related and addictive disorders, post traumatic stress disorder, panic disorder, major depressive disorder with anxious distress, diseases related to modulation of sympathetic outflow including hypertension, pain, headache, cancer.

21. A pharmaceutical composition comprising the compounds of any preceding claim a pharmaceutically acceptable excipient.

22. A method of producing a compound of claim 1, the method comprising an alkylation or Mitsonobu coupling as shown: [chemical structure]
